(19) United States
(12) Patent Application Publication
(10) Pub. No.: US 2026/0354923 A1
(43) Pub. Date: Nov. 12, 2026

(54) DEVICES FOR CONTROLLING AN ENDOVASCULAR SYSTEM

(71) Applicant: UAB Inovatyvi Medicina, Kaunas (LT)

(72) Inventors: Edvardas SATKAUSKAS, Kaunas (LT); Vaidas LABUNSKAS, Kaunas (LT); Vilius DAMBRAUSKAS, Kaunas (LT); Evaldas KALVAITIS, Kaunas (LT)

(73) Assignee: UAB Inovatyvi Medicina, Kaunas (LT)

(21) Appl. No.: 19/121,704

(22) PCT Filed: Oct. 26, 2023
(86) PCT No.: PCT/EP2023/079884

§ 371 Date: Apr. 16, 2025

(30) Foreign Application Priority Data
Dec. 15, 2022 (DE) 102022133445.7

(51) Int. Cl. A61B 34/00; A61B 34/30; A61B 34/35; A61M 25/01

(52) U.S. Cl. CPC A61B 34/76; A61B 34/25; A61B 34/35; A61B 2034/303; A61M 25/0113

(57) ABSTRACT

A human control unit for manipulating a remote elongated medical member, the human control unit comprising: a control unit comprising a transmitter and a receiver; a local elongated member configured to be manipulated by a user of the human control unit; a gripping unit configured to grip the local elongated member; and a device configured to enable and disable the transmitter.

### FIELD OF THE INVENTION

The present invention generally relates to an apparatus for controlling movement of an elongated member of an endovascular system comprising a first and a second module, a movement unit, a gripping unit, a rotation unit and a plurality of modes of operation. Furthermore, the present invention generally relates to systems for controlling a plurality of elongated members, a system for controlling an elongated member and a human control unit (meaning a control unit controllable by a human) for manipulating a remote elongated medical member

### BACKGROUND TO THE INVENTION

Endovascular specialists (for example (endo-)vascular surgeons, (interventional) cardiologists, (interventional) radiologists etc.) train, practice and develop intuitive skills to handle surgical tools. The mental imagery of skills of physicians also evolves by correlating their actions and responses of surgical tools within the human anatomy. An endovascular surgeon is generally guided by two senses: visual feedback from the imaging devices and reaction force feedback via the tool. Perception-action-visualization abilities of surgeons are fine-tuned to a level where their surgical decisions are made even without observing their hand gestures.

Currently, existing robotic systems are focused exclusively on imaging feedback, but have ignored the other source of information: tactile feedback from surgical tools. Instrument controls using a joystick and a PC interface are closer to videogame controllers than control of surgical instruments and leave vascular surgeons with less feedback information which is available performing the procedure manually.

The inventors have realized that existing vascular robotic systems are controlled via a computer interface, in contrast to what vascular surgeons are trained to do with, for example, guidewires and catheters.

There is therefore a need for improvements of (endo-) vascular robotic systems.

### SUMMARY OF THE INVENTION

The invention is set out in the independent claims. Preferred embodiments of the invention are set out in the dependent claims.

According to a first aspect, we describe a medical apparatus for controlling movement of a first elongated medical member, the apparatus comprising: a first module and a second module, wherein each of the first and second module comprises a corresponding, respective opening for the first elongated medical member to be passed through the first and second modules, respectively; a movement unit configured to move at least one of the first and second modules in a first direction and in a second direction, wherein the first direction is opposite to the second direction; wherein at least one of the first and second modules comprises a first gripping unit configured to grip the first elongated medical member passing through the opening of the respective module; wherein at least one of the first and second modules comprises a rotation unit configured to rotate the first elongated medical member, passing through the opening of the respective module, about a longitudinal axis of the first elongated medical member; wherein the apparatus is configured to operate in a plurality of different modes of operation comprising: a) a first mode of operation comprising the first elongated medical member being gripped by the first gripping unit of at least one of the first and second modules and the movement unit moving the at least one of the first and second modules comprising the first gripping unit in the first direction; b) a second mode of operation comprising the first elongated medical member being gripped by the first gripping unit of at least one of the first and second modules and the movement unit moving the at least one of the first and second modules comprising the first gripping unit in the second direction; c) a third mode of operation comprising the first elongated medical member being gripped by the first gripping unit of at least one of the first and second modules and the rotation unit rotating the first elongated medical member about the longitudinal axis of the first elongated medical member; and d) a fourth mode of operation comprising the first or second mode of operation executed simultaneously with the third mode of operation.

The apparatus may be of any suitable design and/or shape as long as it comprises the above-mentioned features. This may allow for the apparatus to be modified to the situation and/or environment said apparatus is in.

The first and second modules may also be of any suitable design and/or shape that allows for the apparatus to control the movement of the first elongated medical member. For example, the first and/or second modules may be cuboidal, cylindrical, conical, prismatic or any other suitable bespoke shape. The first and second modules may have different designs and/or shapes from the other module. This may allow for the apparatus to be modified to the situation and/or environment said apparatus is in.

The first and second directions may be directions relative to the at least one module. For example, the first and second directions may be in relation to the longitudinal or axial axes of the at least one module. In some examples, the first and second directions may relate to the longitudinal or axial axes of the first elongated medical member. If both the first and second modules comprise the movement unit, the first and second modules may move in the same first and second directions or alternatively, they may move in different first and second directions. For example, the movement of the first module may be in relation to the axial axis of the first module while the second module may move in relation to the longitudinal axis of the first elongated medical member. This may allow for the elongated member to be manipulated in the wanted way. In some examples, the movement unit is comprised in the first and/or second module.

The opening may be of any suitable cross-section and diameter that allows for the first elongated medical member to be passed through the first and second modules. For example, the opening may be circular, quadrilateral, triangular, or another bespoke shape. In some examples, the cross-section of the opening is the same the whole way through the first and/or second module. In some examples, the diameter of the opening may be increase or reduce as the opening passes through the first and/or second module. In some examples, the cross-section of the opening may change as it passes through the first and/or second module. For example, the opening may be circular at one side of the first and/or second module and the cross-section of the opening may alter so that the opening is quadrilateral on the second side of the first and/or second modules, wherein the first side is opposite to the second side. In some examples, the second side is not opposite to the first side. This may allow for the elongated member to be held in a sufficient manner so that the elongated member does not slip/fall out of the opening.

The gripping unit will be described in more detail below. In particular, in addition to the below description of the gripping unit, the gripping unit may be configured to grip the first elongated medical member via reducing a size of the opening of the module comprising the first gripping unit. In some examples, a size of only a portion of the opening is reduced.

The rotation unit is preferably coupled, either directly or indirectly, to the first elongated medical member and is configured to rotate the first elongated medical member passing through the opening of the first and/or second module. The rotation unit may comprise a set of gears and/or a timing pulley and belt coupled to a motor and/or a bearing and/or a shaft and/or any other suitable component that is configurable to rotate the first elongated medical member. This may allow for the elongated member to be rotated in small increments, similar to as if the elongated member were being manipulated by a human hand. In some examples, the rotation unit comprises the gripping unit.

Additionally or alternatively, the first elongated medical member may be configured to be rotated in any other suitable direction by the rotation unit.

The four methods of control may allow for the first elongated medical member to be rotated and/or moved in all directions needed to allow for the first elongated medical member to be fully controlled. This may allow for the elongated member to be manipulated by the apparatus in such a way that it is like being manipulated by a human hand. This may eliminate the need for the surgeon to be in the room where the apparatus is during the function of the apparatus. In some examples, the apparatus is configured to perform only some modes of operation, any combination of modes of operation, or all of the above-mentioned modes of operation.

In some examples, the first gripping unit is configured to be part of a cassette, wherein the cassette comprises the opening suitable for the first elongated medical member. This may allow for the gripping unit and opening to be swapped out should the geometry of the elongated within the opening change and allow for the cassette to be changed should the opening and/or gripping unit need to be repaired. This may allow for quick exchange of the gripping unit and/or opening and allow for the apparatus to be used in conjunction with a wide range of elongated instruments. The cassette is preferably removeable and interchangeable with other cassettes comprising gripping units and openings.

In some examples, the first gripping unit is rotatable by the rotation unit, and wherein the first elongated medical member is rotatable about its longitudinal axis as the first elongated medical member is gripped by the first gripping unit and the first gripping unit is rotated by the rotation unit. The rotation unit may be directly or indirectly coupled to the gripping unit via a set of gears coupled to a motor and/or a bearing and/or a shaft and/or any other suitable component that is configurable to rotate the gripping unit such as, for example, a timing pulley and belt coupled to a motor. This may ensure that the elongated member does not slip/fall out of the opening during the rotation of the elongated member.

In some examples, the gripping unit is configured to grip the first elongated medical member while at least one of the first and second modules is being moved by its respective movement unit. Again, this may ensure that the elongated member does not slip/fall out of the opening during the movement of the elongated member in the first and second directions.

In some examples, the first gripping unit comprising a first gripping component configured to contact at least a first portion of the first elongated medical member on a first side of the first elongated medical member; a second gripping component configured to contact at least the first portion of the first elongated medical member on a second side of the first elongated medical member, wherein the first side is different from the second side; a guide configured to guide, during a movement of the second gripping component, at least a first portion of the second gripping component; and an actuating component coupled to the second gripping component, wherein the second gripping component is moveable between a first position and a second position based on an actuating force provided to the second gripping component via the actuating component, and wherein at least the first portion of the second gripping component is guideable by the guide during movement of the second gripping component between the first position and the second position; wherein the first gripping component is configured to stay stationary or substantially stationary with respect to the guide, wherein the second gripping component comprises a first surface opposite to a first surface of the first gripping component, wherein, when the second gripping component is in the first position, the first elongated medical member is grippable between the first surface of the first gripping component and the first surface of the second gripping component, and wherein, when the second gripping component is in the second position, the first elongated medical member is not grippable between the first surface of the first gripping component and the first surface of the second gripping component.

The first gripping component is configured to contact at least a first portion of the elongated member on a first side of said elongated member and the second gripping component is configured to contact a second side of said elongated member, wherein the first side is different from the second side. This results in the elongated member being contacted by the gripping components on two different sides, thereby allowing for the elongated member to be gripped by the two gripping components. The gripping components may be any suitable shape which allows for said gripping components to contact at least the first portion of the elongated member such as, for example, cuboidal, prismoidal or a bespoke design. The gripping components may comprise any suitable material, as will be described in more detail below.

The guide may be of any suitable design which allows for the movement of the second gripping component to be guided during movement of said second gripping component. The guide may be U-shaped, L-shaped, two substantially vertical pieces which allow for the second gripping component to be guided, or any other bespoke shape.

The actuating component coupled to the second gripping component allows for the second gripping component to be moved between a first and a second position via an actuating force. The actuating force may be a mechanical force, a resilient force, a gravitational force, a magnetic force or any other type of force, or a combination thereof.

The first gripping component is configured to stay stationary or substantially stationary with respect to the guide. This may allow for the first gripping component to be moved at the same time as the guide should the guide be moved and vice versa. The first gripping component may be directly or indirectly coupled to the guide via any suitable coupling means.

The second gripping component is, as described above, moveable between a first and a second position via an actuating force provided to the second gripping component via the actuating component. When the second gripping component is in the first position, the elongated member is grippable between a first surface of the first gripping component and a first surface of the second gripping component. This allows for the elongated member to be gripped in place should an endovascular specialist, or any person, be using said gripper. This allows for the elongated member to be secured in place. In the second position, the elongated member is not grippable between a first surface of the first gripping component and a first surface of the second gripping component. This allows for the elongated member to be moved within the gripper, for an elongated member to be removed from the gripper entirely or for an elongated member to be inserted into the gripper.

Furthermore, as the elongated member is grippable between a first surface of the first gripping component and a first surface of the second gripping component, this may mean that the elongated member is grippable via a compressive force. This may allow for a more secure gripping and/or securing of the elongated member and may allow for the lifetime of the elongated member to be increased, as it is not subjected to shear or tension forces.

In some examples, the first surface of the first gripping component comprises a first recess configured to accommodate at least the first portion of said first elongated medical member on the first side of said first elongated medical member. The recess may be of any suitable design that allows for at least the first portion of the elongated member to be accommodated. The recess may be U-shaped, semi cylindrical, prismoidal, have varying dimensions or be of any bespoke design. In some examples, the recess comprises a plurality of shapes at different locations, which may correspond to differing sections of the elongated member. This may allow for the first gripping component to be designed in such a way that allows for said first gripping component to be able to accommodate a plurality of different types of elongated member and/or if the elongated member has different cross-sections at different sections. This may improve the versatility of the gripper.

In some examples, the first surface of the second gripping component comprises a second recess configured to accommodate at least the first portion of said first elongated medical member on the second side of said first elongated medical member. The recess may be of any suitable design that allows for at least the first portion of the elongated member to be accommodated. The recess may be U-shaped, semi cylindrical, prismoidal, have varying dimensions or be of any bespoke design. In some examples, the recess comprises a plurality of shapes which may correspond to differing sections of the elongated member. This may allow for the second gripping component to be designed in such a way that allows for said second gripping component to be able to accommodate a plurality of different types of elongated member and/or if the elongated member has different cross-sections at different sections. This may improve the versatility of the gripper.

In some examples, the first surface of the second gripping component comprises a protrusion configured to contact at least the first portion of said first elongated medical member on the second side of said first elongated medical member. The protrusion may be of any suitable design that allows for at least the first portion of the elongated member to be contacted. The protrusion may be cuboidal, semi cylindrical, prismoidal, have varying dimensions or be of any bespoke design. In some examples, the protrusion comprises a plurality of shapes which may correspond to differing sections of the elongated member. This may allow for the second gripping component to be designed in such a way that allows for said second gripping component to be able to contact a plurality of different types of elongated member and/or if the elongated member has different cross-sections at different sections.

In some examples, the actuating component comprises a resilient member, in particular a spring. In some examples, the resilient member exerts a biasing force on the second gripping component, wherein the biasing force biases the second gripping component towards the first position i.e. the gripping position. This may allow for the elongated member to be gripped by the gripper without a user of the gripper needing to constantly keep the second gripping component in the first position. The resilient member may be any suitable resilient member such as, for example, a spring, a rubber band or any other suitable component that provides a resilient force.

In some examples, the guide comprises a through hole configured to receive said first elongated medical member between the first surface of the first gripping component and the first surface of the second gripping component. This may allow for the elongated member to be accommodated by the guide, thereby providing an extra securing method as the elongated member movement of the elongated member may be restricted even when the second gripping component is in the second position i.e. the non-gripping position. It may also allow for a portion of the elongated member to extend beyond the guide and indeed, the gripper. More than one section of the guide may comprise a through hole, thereby allowing for the elongated member to extend beyond the guide, and the gripper, on at least two sides of the guide.

In some examples, upon said movement of the second gripping component, based on the actuating force provided to the second gripping component via the actuating component, the first surface of the second gripping component is moveable towards the first surface of the first gripping component. This may allow for the elongated member to undergo a compressive force when gripped between the first and second gripping components. This may allow for a more secure gripping and/or securing of the elongated member and may allow for the lifetime of the elongated member to be increased as it is not subjected to shear or tension forces.

In some examples, the first gripping component is at least partially housed by the second gripping component. This may allow for a limitation of the travel of the second gripping component. This in turn may elongate the lifespan of the gripper as the components of the gripper may not be overstressed or undergo unwanted stresses or movements.

In some examples, the first and second gripping components are at least partially located within an inner guide, wherein the inner guide is located within the guide, and wherein the inner guide is moveable with respect to the guide. The inner guide, when moving with respect to the guide, may act as a form of suspension and cushioning for the elongated medical member. This may in turn reduce the stress and shear forces experienced by the elongated medical member during use of said member, thereby elongating the lifetime of the elongated medical member.

In some examples, the first and second gripping components are offset from each other along a longitudinal axis of the first elongated medical member, and when the second gripping component moves between the first position and the second position, the second gripping component does not contact the first gripping component. This may allow for the elongated medical member to be gripped in a particularly strong manner as it needs to snake its way through the gripper.

In some examples, the first and second gripping components are arranged in a zipper configuration. This may allow for the elongated medical member to be gripped in a particularly strong manner as it needs to snake its way through the gripper.

In some examples, the first and/or second gripping components comprise a truncated V-shape. This may allow for a reduction in stress and shear forces experienced by the elongated medical member as the force exerted on the elongated medical member is spread over a larger area than when compared to a V-shape. This, in turn, may elongate the lifespan of the elongated medical member.

In some examples, the first elongated medical member is an elongated medical instrument, in particular a catheter, a stent, a catheter balloon, a stent balloon, a thrombectomy device, a coil, a glue system or a guidewire. The use of a catheter, a stent, a catheter balloon, a stent balloon, a thrombectomy device, a coil, a glue system or a guidewire may be particularly useful in the field of endovascular systems.

In some examples, if both the first and second modules comprise movement units, the first and second modules are moveable independent from one another. This may mean that the movement units are activated and/or actuated independent from one another. This may allow for the elongated member to be moved in a more refined way, thereby more closely mirroring the movement of a human hand that would manipulate the elongated member. In some examples, the independent movement may allow for slack in the elongated member between the modules to be reduced, thereby increasing the lifespan of the elongated member as it is not subject to extra tension and/or stress forces. In some examples, the independent movement may ensure that the elongated member does not slip/fall out from the openings of the first and second modules.

In some examples, if both the first and second modules comprise first gripping units, the operation of the first gripping units are independent from one another. This may mean that the gripping units are activated and/or actuated independent from one another. This may allow for the elongated member to be gripped in a more refined way, thereby more closely mirroring the movement of a human hand that would manipulate the elongated member. In some examples, the independent gripping may allow for slack in the elongated member between the modules to be reduced, thereby increasing the lifespan of the elongated member as it is not subject to extra tension and/or stress forces. In some examples, the independent gripping may ensure that the elongated member does not slip/fall out from the openings of the first and second modules.

In some examples, if both the first and second modules comprise rotation units, the operation of the rotation units are independent from one another. This may mean that the rotation units are activated and/or actuated independent from one another. This may allow for the elongated member to be rotated in a more refined way, thereby more closely mirroring the movement of a human hand that would manipulate the elongated member. In some examples, the independent rotation may allow for slack in the elongated member between the modules to be reduced, thereby increasing the lifespan of the elongated member as it is not subject to extra tension and/or stress forces. In some examples, the independent rotation may ensure that the elongated member does not slip/fall out from the openings of the first and second modules.

In some examples, the apparatus further comprises a controller configured to control the first gripping unit and/or the movement unit. This may allow for the gripping unit and/or the movement unit to be active and/or actuated based on instructions executed by the controller. Additionally or alternatively, the controller may be configured to control the rotation unit. The controller may be coupled to the movement unit and/or the first gripping unit and/or the rotation unit via a wired connection and/or via a wireless connection. In some examples, the controller may be able to control the first gripping unit and/or the movement unit so that the apparatus can perform at least one mode of operation, any combination of the modes of operation or all modes of operation mentioned above.

In some examples, the apparatus further comprises a sensor, the sensor comprising a moveable member moveable between a first position and a second position, a resilient force member coupled to or integral to the moveable member, wherein the resilient force member is configured to provide a resilient force, when the moveable member is in the second position, to bias the moveable member towards the first position, and a detection unit configured to detect a change in position of the moveable member from the first position to the second position and/or the second position to the first position.

The moveable member may allow for a user of the sensor to move the member. The moveable member may be of any suitable design. The first position and the second position may be different positions. In some examples, the moveable member is coupled to an endovascular medical instrument such as a guidewire and/or a catheter, or any other suitable instrument. In some examples, the moveable member is coupled to any suitable medical instrument. The coupling may allow the user to receive real time haptic feedback from the resilient force member.

The resilient force member may be an integral part of the moveable member and/or couplable to the moveable member. The resilient force member may provide haptic feedback to a user of the sensor. The resilient force member may be of any suitable design. In some examples, the resilient force member is configured to provide a resilient force, when the moveable member is in the second position, to bias the moveable member towards the first position. In some examples, the resilient force member is configured to provide a resilient force, when the moveable member is in the first position, to bias the moveable member towards the second position. The biasing position may be any suitable position.

The detection unit may allow for a movement of the moveable member to be detected. The detection unit may transmit the detected movements to an external source, such as, for example, a controller and/or computer and/or a server and/or a second endovascular robotic system. The detection unit may use optical means and/or magnetic field means and/or any other suitable means to detect the movement of the moveable member from the first position to the second position and/or the second position to the first position.

In some examples, the detection unit comprises an optical unit comprising a light source for emitting light and a light sensor for detecting the light emitted by the light source, wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor, and wherein a first amount of the emitted light which is blockable by the first portion of the moveable member in the optical path between the light source and the light sensor is different between the moveable member being in the first position and the moveable member being in the second position, respectively. The optical unit may comprise any suitable light source and any suitable light sensor. The light sensor may be able to sense the amount of light blocked by the moveable member and/or the area in which the moveable member blocks the light-the optical unit may be configured to determine the amount of light which is blocked based on the total amount of light emitted by the light source and the amount of light which is detected by the light sensor, as will be outlined further below. The amount of light blocked by the moveable member may be in some examples different between the first and second positions and in some examples, not different. The optical unit may be able to determine the position and/or orientation of the moveable member in the optical path. In some examples, the light sensor is a linear camera with a 1500×1 pixel array. The pixel array may sense the position and/or amount of the blocked light.

In some examples, the optical unit further comprises a lens arranged in the optical path between the light source and the light sensor, and wherein the lens is configured to disseminate the light emitted by the light source. This in turn may allow for a greater movement of the moveable member in the optical path so that the range of what can be detected is enhanced. It may also allow for the amount of blocked light to be sensed more accurately by the light sensor as the proportion of light in the optical path blocked by the moveable member is smaller. This in turn may lead to a more precise determination of the position and/or orientation of the moveable member.

In some examples, the light source comprises a laser diode. This may allow for a particularly effective optical unit as laser diodes provide a constant light intensity at a constant wavelength, thereby leading to a more accurate light sensor. Additionally or alternatively, any other suitable light source may be used.

In some examples, the sensor is configured to transmit data relating to sensed light stemming from the light source to an external receiver. The external receiver may be any suitable receiver. The external receiver may receive the data via wired and/or wireless means. In some examples, the data is transmitted via a RS232/RS485 physical connection with a proprietary protocol.

In some examples, the first elongated medical member is removable from the apparatus while the apparatus is in use, wherein the first elongated medical member is removeable by retracting the first elongated medical member through the openings of the first and second modules. This may allow for the elongated member to be replaced during the use of the apparatus. This may allow for damaged elongated members to be replaced and/or for a different elongated member to be used should the situation change.

In some examples, the cassette is removeable and replaceable based on the first elongated medical member in the opening. This may allow for the gripping unit and opening to be swapped out should the geometry of the elongated within the opening change and allow for the cassette to be changed should the opening and/or gripping unit need to be repaired. This may allow for quick exchange of the gripping unit and/or opening and allow for the apparatus to be used in conjunction with a wide range of elongated instruments.

In some examples, the rotation unit comprises a gear and the first elongated medical member is couplable to said gear, and wherein the first elongated medical member is rotatable by the gear through an unlimited rotational angle. The elongated member may be directly or indirectly coupled to the gear. This may allow for the elongated member to be manipulated in ways that aid with the use of the apparatus that are not possible when the elongated member is being manipulated by a human hand. This, therefore, may allow for the apparatus to be used in a greater range of applications when compared to human manipulation. This may also allow for a more refined rotation of the elongated member, thereby mirroring the manipulation of the elongated member by a human.

In some examples, the first elongated medical member is an over-the-wire type first elongated medical member. The skilled person understands that an over-the-wire type elongated medical member allows for an endovascular procedure to be performed from start to finish. In some examples, a rapid exchange, or RX system may be used in conjunction with the over-the-wire type member and allow for a quicker procedure and/or additional help regarding additional devices/members. For example, for an RX procedure, a surgeon may start with an over-the-wire type member, then switch to an RX type member, and then switch back to an over-the-wire type member.

According to a second aspect, we describe a system for controlling a plurality of first elongated medical members, the system comprising: a plurality of apparatuses as described above; wherein each apparatus is for controlling movement of a separate first elongated medical member not controlled by any of the other of the plurality of apparatuses.

This may allow for multiple instruments to be manipulated simultaneously, thereby leading to a more accurate representation of a human manipulating a plurality of members during use of said members. In some examples, the first instruments may not be the same. The use of a catheter, a stent, a catheter balloon, a stent balloon, a thrombectomy device, a coil, a glue system or a guidewire may the first member of the plurality of members may be a catheter, a second member of the plurality may be a catheter, a stent, a balloon, a stent balloon, a thrombectomy device, a coil or a glue system device, and a third member may be a guidewire, a thrombectomy device, a coil or a glue system device.

In some examples, wherein in the modes of operation described above, when the first elongated medical member is moveable and/or rotatable by at least one of the first and second modules of a first apparatus, at least one of the first and second modules of a second apparatus moves in relation to the at least one of the first and second modules of the first apparatus. This may allow for slack in the elongated member to be reduced and may allow for the lifetime of the elongated member to be increased, as it is not subjected to shear or tension forces. This may also allow for the elongated member to be kept within the openings of the modules and allow for the elongated member to not slip/fall out of the openings. This also may allow for greater refinement of the movement of the elongated member while the system is in use and allow for the movement of the elongated member to more closely mirror that of an elongated member that is manipulated by a human hand.

In some examples, wherein if the first elongated medical member of the first apparatus is gripped, via the first gripping unit, by only one of the first and second modules of the first apparatus, the other one of the first and second modules of the first apparatus is moveable away from the module gripping the first elongated medical member, and at least one of the first and second modules of the second apparatus moves in response to the movement of the module of the first apparatus not gripping the first elongated medical member. This may allow for the module to move so that the module can regrip the elongated member in a place further away from the other one of the modules in the apparatus, thereby allowing to the elongated member to be moved in the first/second direction along its entire length. This may allow for slack in the elongated member to be reduced and may allow for the lifetime of the elongated member to be increased, as it is not subjected to shear or tension forces. This may also allow for the elongated member to be kept within the openings of the modules and allow for the elongated member to not slip/fall out of the openings. This also may allow for greater refinement of the movement of the elongated member while the system is in use and allow for the movement of the elongated member to more closely mirror that of an elongated member that is manipulated by a human hand.

According to a third aspect, we describe a system for controlling movement of a first elongated medical member, the system comprising: the apparatus as described above; and a human control unit comprising a control unit and a second elongated medical member configured to be manipulated by a human; wherein the human control unit is located at a first location and the apparatus is located at a second location; and wherein the first and second locations are different locations.

The human control unit may be located at a location remote from the apparatus and be coupled to the apparatus vis wired and/or wireless means. The second elongated medical member is configured to be manipulated by a human. In some examples, the second elongated medical member is a replica of the first elongated medical member, or is a representation of the first elongated medical member. This may allow for the human to have a better understanding of the feel of the first elongated medical member as the second elongated medical member comprises, preferably, the same materials and dimensions as the first elongated medical member.

In some examples, the human control unit comprises a second gripping unit configured to grip the second elongated medical member. This second gripping unit may permanently grip the second elongated medical member. This may reduce the chance of the second elongated medical member slipping/falling out from the gripping unit. In some examples, the design of the second gripping unit is identical to the first gripping unit. In some examples, the second gripping unit may ungrip the second elongated medical member for repair and/or cleaning purposes, but grip the second elongated medical member during use of the human control unit.

In some examples, the control unit is configured to transmit a first control signal to the controller of the apparatus, wherein the first control signal comprises information on the manipulation of the second elongated medical member. The information may relate to the distance the second elongated medical member has been moved in a predetermined direction, a rotation of the second elongated medical member, a force that has been applied on the second elongated medical member or any other suitable information. This may allow for the apparatus to receive important information about the manipulation of the second elongated medical member. In some examples, the information may comprise a measured location of the first and/or second gripping unit with respect to the first and second elongated medical members, respectively. This measurement may comprise a time stamp of a particular event or moment, and comprise information on a distance and/or rotation of the first and/or second gripping unit with respect to the first and second elongated medical members and/or a force being measured by the sensor described herein. In some examples, the information may be transmitted using a cyclic redundancy check, CRC, to detect possible errors. Additionally or alternatively, any other error detection method can be used. In some examples, the distance, i.e. a linear distance, is measured via a linear encoder coupled to the linear gear and/or motor that provides the linear movement. In some examples, the rotation is measured via a rotational encoder coupled to the rotational gear and/or motor that provides the rotational movement. In some examples, the force is measured by the sensor, as described herein.

In some examples, the controller of the apparatus controls the first elongated medical member in a manner proportional to the manipulation of the second elongated medical member, wherein the controller controls the first elongated medical member based on the received first control signal from the control unit of the human control unit. This may allow for the remote manipulation of the first elongated medical member via the manipulation, by a human, of the second elongated medical member.

In some examples, the human control unit further comprises a haptic feedback unit configured to provide haptic feedback to the human manipulating the second elongated medical member based on a sensor unit reading, wherein the haptic feedback is based on the sensor unit reading. This may allow human at the human control unit to feel what is happening at the apparatus and realize if the movement of the first elongated medical member has been blocked, for example, as the haptic feedback may be suddenly greatly increased. In some examples, the sensor unit may comprise the sensor as described herein.

In some examples, the human control unit further comprises a pedal actuatable by the human, wherein in a first position, the control unit is configured to transmit the first control signal and in a second position, the control unit is configured not to transmit the first control signal, wherein the first position is different from the second position. This pedal may act as a “dead man's switch” and ensure that unintended movements of the second elongated medical member are not transmitted to the apparatus via the first control signal. This, in turn, improves the safety of the system.

In some examples, the human control unit further comprises a touchable device configured to be touchable by the human, wherein upon a first touch of the touchable device, the control unit is configured to transmit the first control signal and upon a second touch, the control unit is configured not to transmit the first control signal, wherein the first and second touches occur at two different temporal instances. This may also act as a “dead man's switch” and ensure that unintended movements of the second elongated medical member are not transmitted to the apparatus via the first control signal.

In some examples, the human control device further comprises a visual indicator configured to indicate that the first control signal is not being transmitted. This may allow for the human to easily see that the movements of the second elongated medical member are not being transmitted, and so, the human is free to move the second elongated medical member. In some examples, if the second elongated medical member is moved and the first control signals are not being transmitted, an audio and/or a vibration indicator may additionally, or alternatively, be used.

In some examples, upon the first touch, information on only an axial or rotational manipulation of the second elongated medical member is transmitted and upon the second touch, information on only an axial or rotational manipulation of the second elongated medical member is not transmitted. This may be particularly helpful if the human wants to move the first elongated medical member, via the first control signal, in only the first and second directions or wants to only rotate the first elongated medical member. This may further improve safety of the system as unintended movements are not transmitted to the apparatus, via the first control signal.

In some examples, the touchable device is a button and/or a lever and/or a touchscreen. This may allow for the human to easily realize if the touchable device has been touched once or twice. In some examples, the touchable device comprises the visual indicator described above. The touchable device is preferably within easy reach of the human so that the human does not need to let go of the second elongated medical member in order to touch the touchable device.

According to a fourth aspect, we describe a system for controlling a plurality of first elongated medical members, the system comprising: the system of the second aspect; and a human control unit comprising a control unit and at least a second elongated medical member and a third elongated medical member, both configured to be manipulated by a human; wherein the human control unit is located at a first location and the apparatus is located at a second location; and wherein the first and second locations are different locations. The system is described here as being of the second aspect, but the system could be any type of suitable system for which a plurality of elongated members needs to be controlled.

In some examples, the human control unit comprises a second and a third gripping unit configured to grip at least the second and the third elongated medical members, respectively. This second and third gripping units may permanently grip the second and third elongated medical members, respectively. This may reduce the chance of the second and third elongated medical members slipping/falling out from the gripping units. In some examples, the design of the second and/or third gripping units are identical to the first gripping unit. In some examples, the second and/or third gripping units may ungrip the second and/or third elongated medical member, respectively, for repair and/or cleaning purposes, but grip the second and/or third elongated medical members during use of the human control unit.

In some examples, the control unit is configured to transmit a first control signal to the main controller, wherein the first control signal comprises information on the manipulation of at least the second elongated medical member and the third elongated medical member. The information may relate to the distance the second and/or third elongated medical members have been moved in a predetermined direction, a rotation of the second and/or third elongated medical members, a force that has been applied on the second and/or third elongated medical member or any other suitable information. This may allow for the system to receive important information about the manipulation of the second and/or third elongated medical members. In some examples, the information may comprise a measured location of the first and/or second gripping unit with respect to the first and second elongated medical members, respectively. This measurement may comprise a time stamp of a particular event or moment, and comprise information on a distance and/or rotation of the first and/or second gripping unit with respect to the first and second elongated medical members and/or a force being measured by the sensor described herein. In some examples, the information may be transmitted using a cyclic redundancy check, CRC, to detect possible errors. Additionally or alternatively, any other error detection method can be used. In some examples, the distance, i.e. a linear distance, is measured via a linear encoder coupled to the linear gear and/or motor that provides the linear movement. In some examples, the rotation is measured via a rotational encoder coupled to the rotational gear and/or motor that provides the rotational movement. In some examples, the force is measured by the sensor, as described herein.

In some examples, the main controller controls the first elongated medical member in a manner proportional to the manipulation of the second elongated medical member, and a further separate elongated member in a manner proportional to the manipulation of the third elongated medical member, wherein the controller controls the first and the further elongated members based on the received first control signal from the control unit of the human control unit. This may allow for the remote manipulation of the first and/or further elongated members via the manipulation, by a human, of the second and/or third elongated medical members.

In some examples, the human control unit further comprises a haptic feedback unit configured to provide haptic feedback to the human manipulating at least the second and third elongated medical members based on a second control signal transmitted by the detection unit of the sensor, wherein the haptic feedback is proportional to data relating to sensed data of the sensor. This may allow human at the human control unit to feel what is happening at the system and realize if the movement of the first elongated medical member has been blocked, for example, as the haptic feedback may be suddenly greatly increased.

In some examples, the human control unit further comprises a pedal actuatable by the human, wherein in a first position, the control unit is configured to transmit the first control signal and in a second position, the control unit is configured not to transmit the first control signal, wherein the first position is different from the second position. This pedal may act as a “dead man's switch” and ensure that unintended movements of the second and/or third elongated medical members are not transmitted to the apparatus via the first control signal. This, in turn, improves the safety of the system.

In some examples, the human control unit further comprises a touchable device configured to be touchable by the human, wherein upon a first touch of the touchable device, the control unit is configured to transmit the first control signal and upon a second touch, the control unit is configured not to transmit the first control signal, wherein the first and second touches occur at two different temporal instances. This may also act as a “dead man's switch” and ensure that unintended movements of the second and/or third elongated medical members are not transmitted to the apparatus via the first control signal.

In some examples, upon the first touch, information on only an axial or rotational manipulation of the second and/or third elongated medical member is transmitted and upon the second touch, information on only an axial or rotational manipulation of the second and/or third elongated medical member is not transmitted. This may be particularly helpful if the human wants to move the first elongated medical member, via the first control signal, in only the first and second directions or wants to only rotate the first elongated medical member. This may further improve safety of the system as unintended movements are not transmitted to the system, via the first control signal.

In some examples, the human control device further comprises a visual indicator configured to indicate that the first control signal is not being transmitted. This may allow for the human to easily see that the movements of the second and/or third elongated medical members are not being transmitted, and so, the human is free to move the second ad/or third elongated medical member. In some examples, if the second and/or third elongated medical members are moved and the first control signals are not being transmitted, an audio indicator and/or a vibration may additionally, or alternatively, be used.

In some examples, the touchable device is a button and/or a lever and/or a touchscreen. This may allow for the human to easily realize if the touchable device has been touched once or twice. In some examples, the touchable device comprises the visual indicator described above. The touchable device is preferably within easy reach of the human so that the human does not need to let go of the second and/or third elongated medical members in order to touch the touchable device.

In the above, as an example, the user side, i.e. the side of the human control unit, the user may start to move the second and/or third, i.e. local, elongated medical member. The second and/or third elongated member is preferably fixed with respect to its respective gripping unit, and the gripping unit is preferably coupled to a spring and/or a linear drive and/or a rotational drive to allow for the movement and rotation of the gripping unit. To move the second and/or third elongated member, the user exerts a force sufficient to overcome the biasing force of the spring and move the second and/or third elongated member a particular distance. For example, a 1 N force may cause a 1 mm movement until the force between the spring and the user's exerted force become equal. At the same time, on the patient side of the system, the first, i.e. remote, elongated medical member may be also moved 1 mm, and stops moving when the forces on the user side equalize.

As an additional example, a continuous, unbalanced force of 1 N is assumed at the user side. In this example, the first elongated medical member is moved in a similar manner as described above, but encounters a blockage and so, the readings of the patient side sensor becomes 1 N. This may indicate to the user that there is a blockage at the patient side of the system. If the user keeps increasing the exerted force to, for example, 3 N, the elongated members of the user and patient sides translate until the forces balance, so until the force on the patent side also reaches 3 N.

At this moment, if the user releases the second and/or third elongated medical member, due to the spring coupled to the gripping unit, the second and/or third elongated medical member moves back at its zero point with a balanced force of ON, i.e. its predetermined zero point. Resultantly, the first elongated medical member also moves to its predetermined zero point. This is to say that the user tries to exert a force on the second and/or third elongated medical member and feels the resistive force from the spring. If a dead man's switch is used, as described in the present application, the system stays in 3 N=3 N situation when the switch is in a first position, and when the switch is moved to the second position, the first elongated medical member and the second and/or third elongated medical member would revert to their respective zero points. In some examples, if the dead man's switch is in the first position holding the elongated medical instruments in place, this switch could be overcome by the user exerting more force on the second and/or third elongated medical member if the user wants to take the control of the member from the system. This additional force may be altered depending on the situation the system is used in and/or user preference.

The above system may also be used in any of the aspects described herein. Additionally or alternatively, although the above example is described in relation to force being exerted on the second and/or third elongated medical member on the user side, the same principles apply to a force being applied to the first elongated medical member on the patient side.

According to a fifth aspect, we describe a human control unit for manipulating a remote elongated medical member, the human control unit comprising: a control unit comprising a transmitter and a receiver; a local elongated member configured to be manipulated by a user of the human control unit; a gripping unit configured to grip the local elongated member; and a device configured to enable and disable the transmitter. The control unit may, preferably, be the same control unit, or a control unit of the same design, as described above in relation to the third and fourth aspects. The local and/or the remote elongated member may be the same, or of the same design and/or variety, as the first and/or second elongated medical members described above. The gripping unit may be the same, or the same design as, the gripping unit disclosed above in relation to the first, second and third aspects. In some examples, the control unit comprises a transmitter and/or a receiver.

In some examples, the device is a button and/or a lever and/or a touchscreen, wherein upon a first touch of the device, the transmitter is enabled and upon a second touch of the device, the transmitter is disabled, wherein the first and second touches occur at two different temporal instances. The device may act as a “dead man's switch” and ensure that unintended movements of the elongated member is not transmitted to the apparatus. This may also allow for the human to easily realize if the touchable device has been touched once or twice. In some examples, the touchable device comprises the visual indicator described above. The touchable device is preferably within easy reach of the human so that the human does not need to let go of the second and/or third elongated medical members in order to touch the touchable device.

In some examples, the human control unit further comprises a display configured to display, to the user, a state of the remote elongated member. The state of the remote elongated member may relate to a distance in a predetermined direction it has been moved from a zero point and/or a rotation angle of the remote elongated member from a predetermined zero point and/or a force being exerted on the remote elongated member and/or a rate of change of speed and/or angle of the remote elongated member as it is being rotated by the user and/or any other suitable state, such as, for example, a name and/or a diameter of the remote elongated member, that can be displayed to the user. This may allow the user to determine a state of the local elongated member and get a better idea of what the user has done so far and may help the user plan future steps. This also may allow for the user to see if there are any problems with the local elongated member and/or gripping unit if the user recognizes that the states on the display do not match up with what they have done. Additionally or alternatively, a state of the local elongated member may be displayed.

In some examples, the state comprises a distance the remote elongated member has been manipulated and/or a rotational angle through which the remote elongated member has been manipulated, from a predetermined position, as mentioned above.

In some examples, the transmitter is configured to transmit a first control signal to an external device comprising the remote elongated medical member, wherein the first control signal comprises information on the manipulation of the local elongated member, wherein the human control unit is located in a first location and the external device is located at a second location, wherein the first and second locations are different locations. This may allow for the external device to receive important information about the manipulation of the local elongated member. The two locations being separate locations may eliminate the need for the surgeon to be in the room where the external device is during the function of the human control unit.

In some examples, the external device receives the first control signal and the external device manipulates the remote elongated medical member based on the received first control signal. This may allow for the remote manipulation of the remote elongated medical member via the manipulation, by a human, of the local elongated member at the human control unit.

In some examples, the control unit is configured to receive, via the receiver, a second control signal, from the external device, wherein the second control signal comprises information on the remote elongated medical member. This may allow for the human control unit to receive information on the status of the remote elongated medical member. In some examples, this status may be shown on the display. This may therefore improve the safety of the human control unit as the user can see, on the display, if the remote elongated medical member is malfunctioning and/or if the external device is not receiving the first control signal properly. The receiver may be part of the control unit, or may alternatively be located in a separate part of the human control unit. In some examples, the information may comprise a measured location of the first and/or second gripping unit with respect to the first and second elongated medical members, respectively. This measurement may comprise a time stamp of a particular event or moment, and comprise information on a distance and/or rotation of the first and/or second gripping unit with respect to the first and second elongated medical members and/or a force being measured by the sensor described herein. In some examples, the information may be transmitted using a cyclic redundancy check, CRC, to detect possible errors. Additionally or alternatively, any other error detection method can be used. In some examples, the distance, i.e. a linear distance, is measured via a linear encoder coupled to the linear gear and/or motor that provides the linear movement. In some examples, the rotation is measured via a rotational encoder coupled to the rotational gear and/or motor that provides the rotational movement. In some examples, the force is measured by the sensor, as described herein. In some examples, the control unit may receive one or more of the above-mentioned information possibilities from at least one of the remote elongated medical members, should there be a plurality of remote elongated medical members.

In some examples, the human control unit further comprises a haptic feedback unit configured to provide haptic feedback to the user of the human control unit based on the received second control signal. This may allow user at the human control unit to feel what is happening at the external device and realize if the movement of the remote elongated medical member has been blocked, for example, as the haptic feedback may be suddenly greatly increased.

In some examples, the gripping unit is moveable and/or rotatable based on the manipulation of the local elongated member. The local elongated member is preferably fixed with respect to the gripping unit, and the gripping unit is coupled to a spring and/or a linear drive and/or a rotational drive to allow for the movement and rotation of the gripping unit.

In some examples, the gripping unit is moveable and/or rotatable based on the received second control signal. The local elongated member is preferably fixed with respect to the gripping unit, and the gripping unit is coupled to a spring and/or a linear drive and/or a rotational drive to allow for the movement and rotation of the gripping unit.

In some examples, the human control unit comprises a plurality of local elongated members and the human control unit is for manipulating a plurality of remote elongated medical members. This may allow for multiple elongated members to be manipulated simultaneously, thereby leading to a more accurate representation of a human manipulating a plurality of members during use of said members. In some examples, the local and/or remote elongated members may not be the same. The use of a catheter, a stent, a catheter balloon, a stent balloon, a thrombectomy device, a coil, a glue system or a guidewire may be particularly useful in the field of endovascular systems. For example, the first member of the plurality of remote members may be a catheter, a second member of the plurality may be a stent and a third member may be a guidewire.

In some examples, the plurality of local elongated members is telescopically collapsible inside one another. This may allow for the apparatuses to be placed closer to one another during use and/or storage and/or transportation of the system as space for each of the plurality of members is not needed, but only for the longest of the plurality of local members. Additionally, this may allow for, for example, a guidewire and/or catheter to be placed inside another catheter. This, in turn, may allow for manipulation of the guidewire and/or catheter to be made easier as it is already within the another catheter, thereby improving ease of use of the system.

In some examples, there is an external device for each one of the plurality of local elongated members. This may allow for the remote manipulation of a plurality of different remote elongated medical members at a plurality of different remote locations.

In some examples, there is a device for each one of the plurality of local elongated members. This may allow for the first control signal to be transmitted, or not transmitted for each of the plurality of local elongated members individually.

In some examples, the display is configured to display, to the user, a state of each of the plurality of local elongated members. The state of the elongated member may relate to a distance in a predetermined direction it has been moved from a zero point and/or a rotation angle of the local elongated member from a predetermined zero point and/or a force being exerted on the local elongated member and/or a rate of change of speed and/or angle of the local elongated member as it is being rotated by the user and/or any other suitable state, such as, for example, a name and/or a diameter of the remote elongated member, that can be displayed to the user. This may allow the user to determine a state of the local elongated member and get a better idea of what the user has done so far and may help the user plan future steps. This also may allow for the user to see if there are any problems with the local elongated member and/or gripping unit if the user recognizes that the states on the display do not match up with what they have done.

In some examples, the first control signal comprises information on the manipulation of each of the plurality of local elongated members. This may allow for the signals to be transmitted independently from one another to different remote locations. This may allow for the plurality of remote elongated medical members to be manipulated independently from one another.

In some examples, the external device receives the first control signal and the external device manipulates the plurality of remote elongated medical members based on the received first control signal. This may allow for the remote manipulation of the plurality of remote elongated medical members via the manipulation, by a human, of the plurality of elongated members at the human control unit.

In some examples, each of the plurality of local elongated members corresponds to one of the plurality of remote elongated medical members, wherein the number of local elongated members and the number of remote elongated medical members are equal. This may allow for one local elongated member at the human control unit to be linked to a remote elongated medical member. This may allow for each remote elongated medical member to be manipulated independently from the other remote elongated medical members.

In some examples, the human control unit further comprises a plurality of haptic feedback units configured to provide haptic feedback to the user of the human control unit based on the received second control signal, wherein each of the plurality of haptic feedback units is coupled to a different one of the plurality of local elongated members. This may allow user at the human control unit to feel what is happening at the external device(s) and realize if the movement of at least one of the plurality of remote elongated medical members has been blocked, for example, as the haptic feedback may be suddenly greatly increased.

In some examples, the human control unit further comprises a plurality of gripping units, wherein each gripping unit is configured to grip a different one of the plurality of local elongated members. These gripping units may be the same, or the same design as, the gripping unit disclosed above in relation to the first, second and third aspects.

In some examples, at least one of the plurality of gripping units is moveable and/or rotatable based on the manipulation of its respective local elongated member. The local elongated member is preferably fixed with respect to the gripping unit, and the gripping unit is coupled to a spring and/or a linear drive and/or a rotational drive to allow for the movement and rotation of the gripping unit.

In some examples, at least one of the plurality gripping units is moveable and/or rotatable based on the received second control signal. The local elongated member is preferably fixed with respect to the gripping unit, and the gripping unit is coupled to a spring and/or a linear drive and/or a rotational drive to allow for the movement and rotation of the gripping unit.

Any advantages and features described in relation to the any of the above aspects and examples may be realized in any of the other aspects and examples described above.

It is clear to a person skilled in the art that certain features of the system set forth herein may be implemented under use of hardware (circuits), software means, or a combination thereof. The software means can be related to programmed microprocessors or a general computer, an ASIC (Application Specific Integrated Circuit) and/or DSPs (Digital Signal Processors). For example, a processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

Even if some of the aspects described above have been described in reference to any one of the first to fifth aspects, these aspects may also apply to a method (in particular of controlling an elongated member and/or a remote elongated medical member) and vice versa.

### BRIEF DESCRIPTION OF THE DRAWINGS

These and other aspects of the invention will now be further described, by way of example only, with reference to the accompanying figures, wherein like reference numerals refer to like parts, and in which:

FIG. 1 shows a schematic view of an apparatus for controlling movement of a plurality of elongated members according to some example implementations as described herein;

FIG. 2 shows a schematic view of an apparatus for controlling movement of a plurality of elongated members according to some example implementations as described herein;

FIG. 3 shows a schematic view of a human control unit for manipulating a remote elongated medical member according to some example implementations as described herein;

FIG. 4 shows a schematic view of a human control unit for manipulating a remote elongated medical member according to some example implementations as described herein;

FIG. 5 shows a perspective view of a schematic illustration of the gripper according to some example implementations as described herein;

FIG. 6 shows a cross-sectional view of a schematic illustration of parts of the gripper according to some example implementations as

FIG. 7 shows a cut-away view of a schematic illustration of the gripper according to some example implementations as described herein;

FIG. 8 shows a cross-sectional view of a schematic illustration of parts of the gripper according to some example implementations as described herein;

FIG. 9 shows a cross-sectional view of a schematic illustration of parts of the gripper according to some example implementations as described herein;

FIG. 10 shows a schematic block diagram of an endovascular system according to some example implementations as described herein;

FIG. 11a and b show a cross-sectional view of a schematic illustration of parts of the gripper according to some example implementations as described herein;

FIG. 12 shows a cut-away view of a schematic illustration of the gripper according to some example implementations as described herein;

FIG. 13 shows a cut-away view of a schematic illustration of the sensor according to example implementations as described herein;

FIG. 14 shows a perspective view of a schematic illustration of parts of the sensor according to example implementations as described herein;

FIG. 15a and b show schematic block diagrams of the resilient force member according to example implementations as described herein;

FIG. 16 shows a schematic block diagram of the optical unit according to example implementations as described herein;

FIG. 17 shows a schematic block diagram of the light sensor and the processing unit according to some example implementations as described herein;

FIG. 18 shows a cut-away view of a schematic illustration of a sensor according to some example implementations as described herein;

FIG. 19 shows a perspective view of a schematic illustration of parts of the sensor according to some example implementations as described herein;

FIG. 20 shows a perspective view of a schematic illustration of a gripper mechanism according to some example implementations as described herein;

FIG. 21 shows a schematic block diagram of a procedure of detecting the position of the moveable member according to some example implementations as described herein;

FIG. 22 shows a schematic block diagram of an endovascular robotic system according to some example implementations as described herein;

FIG. 23 shows a schematic block diagram of a module according to some example implementations as described herein; and

FIG. 24 shows a schematic block diagram of a system according to some example implementations as described herein.

### DETAILED DESCRIPTION OF THE PREFERRED EMBODIMENTS

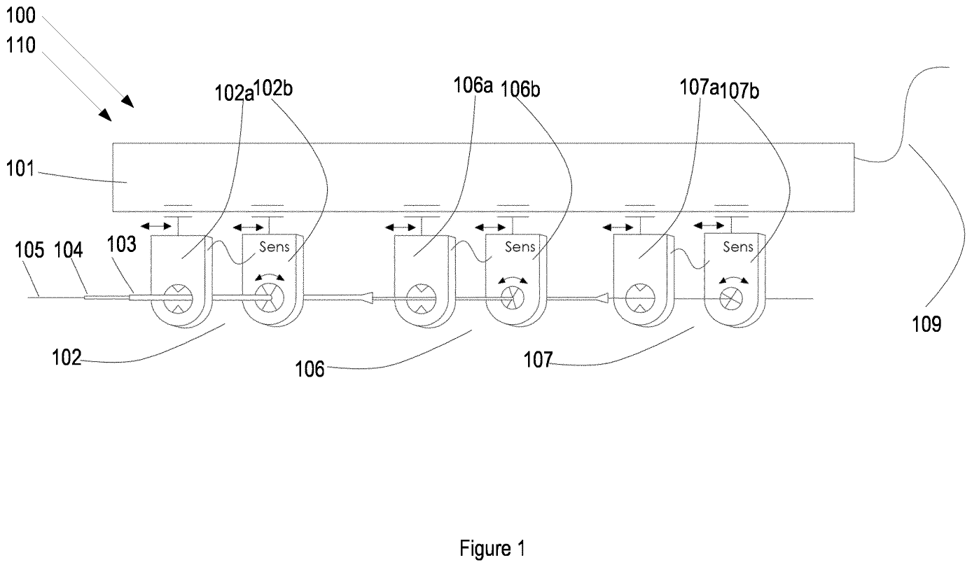

FIG. 1 shows a schematic view of an apparatus for controlling movement of a plurality of elongated members according to some example implementations as described herein.

The apparatus 100 of FIG. 1 comprises a linear drive device 101 with three module pairs 102, 106, 107. In this example, and throughout the present description, reference will be made to three module pairs 102, 106, 107. However, it is to be understood that there may be as many module pairs 102, 106, 107 as are wanted. That is to say, there could be a single module pair 102, 106, 107, two module pairs 102, 106, 107, four module pairs 102, 106, 107 or any other suitable number of module pairs 102, 106, 107.

Each module pair 102, 106, 107 comprises a first module 102A, 106A, 107A and a second module 102B, 106B, 107B. These modules 102A, 102B, 106A, 106B, 107A, 107B will be described in more detail below.

The apparatus 100 may also be referred to as a slave apparatus 110 in the context of the present application. Furthermore, for ease of reference, only the first module pair 102 will be referred to below, but it is understood that the same features and abilities may apply to the second and third module pairs 106, 107. Additionally, terms such as “master apparatus”, “user apparatus”, “surgeon apparatus”, “master module”, “user module”, “surgeon module” and the like, and terms such as “slave apparatus”, “patient apparatus”, “slave module” and “patient module” and the like may be interchangeable and mean the same thing.

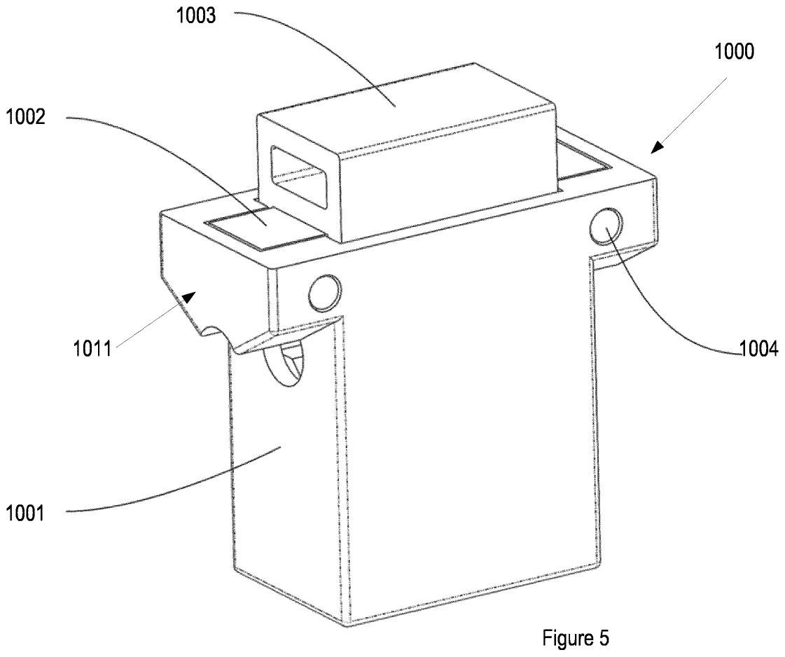

The modules 102A, 102B are devices which hold an elongated medical device 103, 104, 105 and comprise various further elements to actuate the elongated medical devices 103, 104, 105. In particular, each of the modules 102A, 102B is couplable to a track, via the linear drive 101, which allow for the modules 102A, 102B to be moved, via a first motor, along said track to perform a linear movement towards and away from a patient on which the apparatus 100 is located near to. This, in turn, moves the elongated medical device 103, 104, 105 towards and away from the patient. At least one of the modules 102A, 102B within each module pair 102 couplable to an elongated medical device 103, 104, 105 comprises a second motor that allows for the elongated medical device 103, 104, 105 to be rotated, thereby providing a rotational movement of the elongated medical device 103, 104, 105. The linear and rotational movements, via the first and second motors, allow for the elongated medical device 103, 104, 105 to be operated in a realistic manner as if the surgeon was bedside. Furthermore, at least one of the modules 102A, 102B within each module pair 102 couplable to an elongated medical device 103, 104, 105 may comprise a gripper configured to hold the elongated medical device 103, 104, 105 in place. This may allow for the securing of the elongated medical instrument during surgery. The gripper, located within the gripping unit, is described in more detail below with respect to FIGS. 5 to 12.

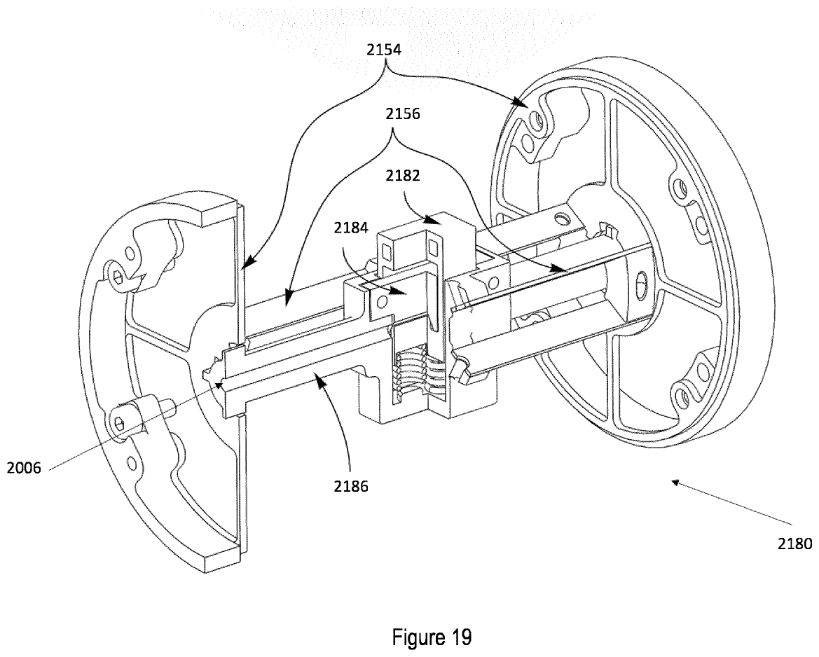

An example of the inside of a module 102A, 102B is shown in FIGS. 19,20 and 23. The first and/or the second motor described above are preferably stepper motors.

The use of stepper motors may allow for the axial and rotational movement of the elongated medical device 103, 104, 105 to be controlled with a high precision, and allow for an improved repeatability of movement. The two motors are also preferably located within the module 102A, 102B. In some examples, the motor that allows for the elongated medical device 103, 104, 105 to be axially translated may be located on the module 102A, 102B, but not within the module 102A, 102B.

The linear drive 101 preferably comprises a rail on a base, wherein the module 102A, 102B axially translates on the rail by the motor that allows for the module 102A 102B and the elongated medical device 103, 104, 105 to be axially translated.

Each module also preferably has four modes of operation. In the first mode of operation, the elongated medical member 103, 104, 105 being gripped by the gripping unit of at least one of the first and second modules 102A, 102B and the movement unit, comprising the axial movement motor, moves the at least one of the first and second modules 102A, 102B comprising the gripping unit in a first direction. In the second mode of operation, the elongated medical member 103, 104, 105 being gripped by the first gripping unit of at least one of the first and second modules 102A, 102B and the movement unit, comprising the axial movement motor, moves the at least one of the first and second modules 102A, 102B comprising the gripping unit in a second direction. In the third mode of operation, the elongated medical member 103, 104, 105 being gripped by the gripping unit of at least one of the first and second modules 102A, 102B and the rotation unit, comprising the rotational movement motor, rotates the elongated medical member 103, 104, 105 about the longitudinal axis of the elongated medical member 103, 104, 105. In the fourth mode of operation, the first or second mode of operation is combined with the third mode of operation.

The first and second directions are, preferably, parallel to the longitudinal axis of the elongated medical member 103, 104, 105. This may allow for the elongated medical member 103, 104, 105 to be advanced and retracted towards and away from the patient.

The above modes also allow for the elongated medical member 103, 104, 105 to be advanced or retracted without also rotating said elongated medical member 103, 104, 105 and may allow for the elongated medical member 103, 104, 105 to be rotated without it being advanced or retracted. It may also allow for the elongated medical member 103, 104, 105 to be simultaneously advanced or retracted, and rotated.

In the example shown in FIG. 1, there are three elongated medical members 103, 104, 105, wherein each module pair 102, 106, 107 controls the movement and rotation of a respective elongated medical member 103, 104, 105. The elongated medical member 103, 104, 105 may be a catheter, a stent, a catheter balloon, a stent balloon, a thrombectomy device, a coil, a glue system or a guidewire. In a preferred example, the elongated medical members 103, 104, 105 are telescopically collapsible within one another, with the outer member 103 being a guidewire, the middle member 104 being a catheter and the inner member 105 being a balloon or stent.

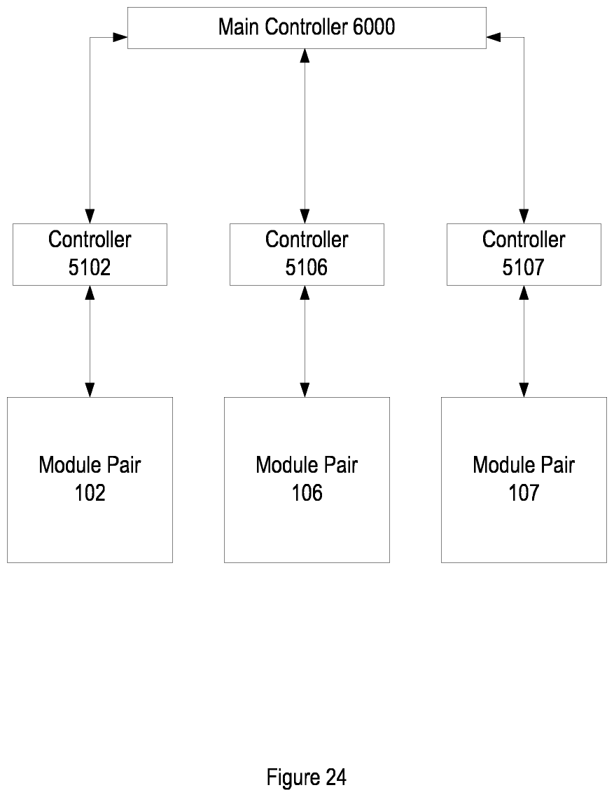

As shown in FIG. 1, as the elongated medical members 103, 104, 105 are being manipulated by the first and/or second modules 102A, 102B, via a controller, see FIG. 24, the first and/or second modules 102A, 102B are axially translated via the axial translation motor in the directions of the arrows shown in FIG. 1. This may allow for the elongated medical members 103, 104, 105 to be advanced and retracted towards and away from the patient. Additionally, the gripper of the gripping unit may be rotated in the direction of the arrows shown in FIG. 1. This may allow for the elongated medical members 103, 104, 105 to be rotated. Additionally, as can be seen in FIG. 1, the second module 102B of each module pair 102 comprises the sensor mentioned above and described below, but it is to be understood that the first module 102A may have this sensor instead, or both the first and second modules 102A, 102B may have the sensor. The controller mentioned here may, preferably, comprise a transmitter and/or a receiver.

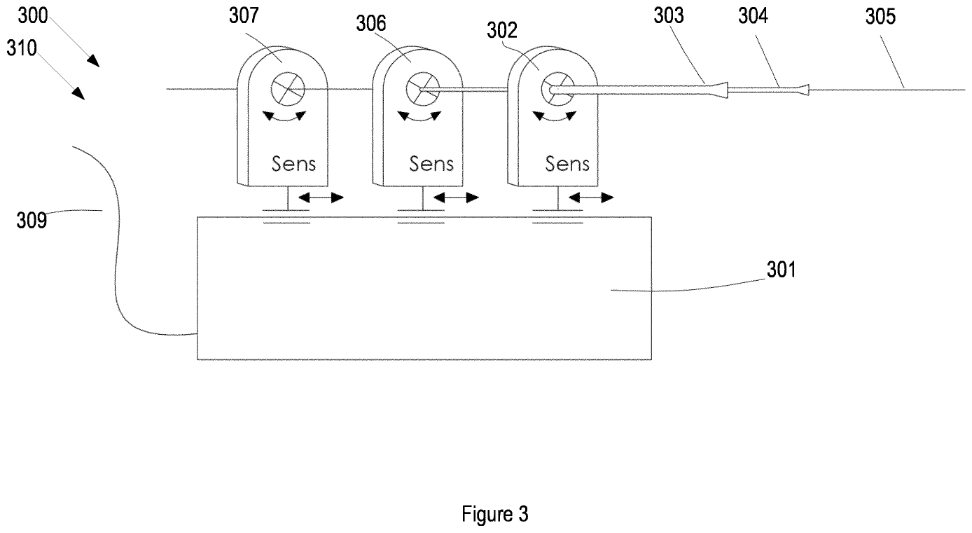

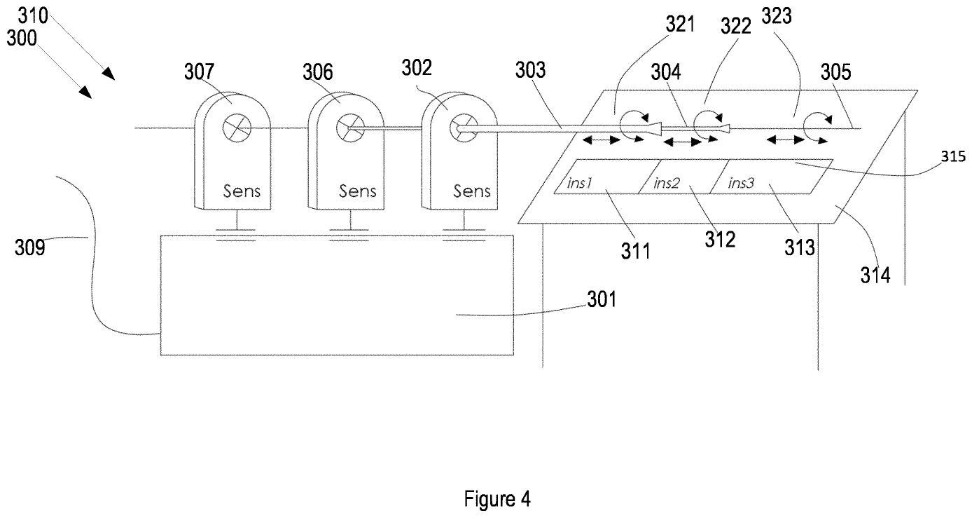

The apparatus 100 further comprises a connector 109 for connection to an external device. This connection 109 may be wired and/or wireless. In some examples, information gathered from the sensors may be transmitted to the external device via this connection 109. Additionally or alternatively, the apparatus 100 may receive instructions from the external device via this connection 109. An example of the external device is described in more detail below with respect to FIGS. 3 and 4.

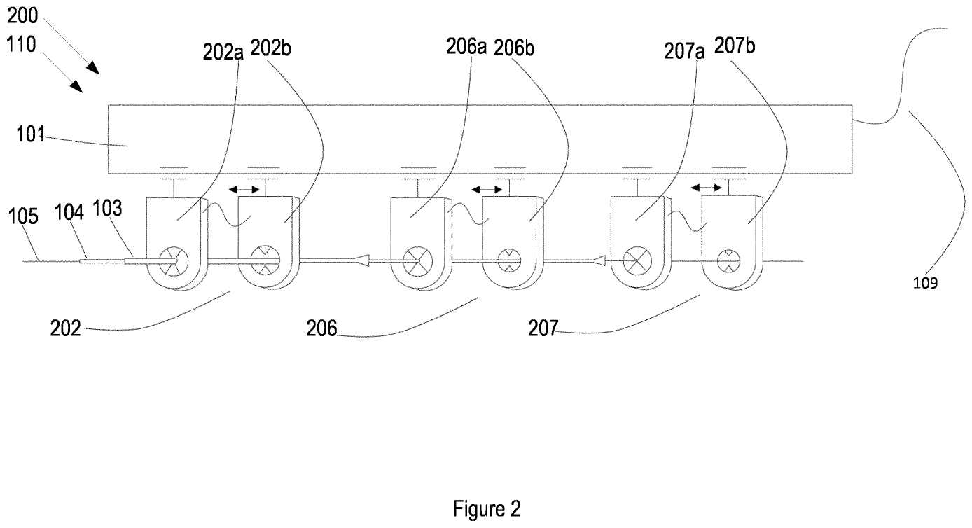

The apparatus 100 preferably uses the “over-the-wire” catheter technique as opposed to a monorail technique. However, the monorail technique is also compatible with the examples of FIGS. 1 and 2. Each of the elongated medical members 103, 104, 105 may be controlled individually via a controller within the first and/or second module 102A, 102B and/or via a main controller in the same of a plurality of module pairs 102, 106, 107 (see FIG. 24). For example, the outer guidewire 103 may be “frozen” in place, i.e. not moveable, by the first and/or second module 102A, 102B, and only the inner catheter 104, 105 may be movable due to manipulation mentioned below in relation to FIGS. 3 and 4. Alternatively, the inner catheter 104, 105 may be “frozen” and the outer guidewire 103 may be manipulated.

In some examples, the apparatus 100 may additionally or alternatively be compatible with rapid exchange (RX) functionality. For the RX instruments, a Y shape adapter may be used, with the adapter being interchangeable based on the application of the apparatus 100 and/or the elongated medical members 103, 104, 105.

The linear drive 101 of the apparatus 100 allows for the modules 102A, 102B to be axially moved parallel to each other, and is also used to support the modules 102A, 102B as a form of a base for the apparatus 100. The modules 102A, 102B may be moved in axes parallel to one another or, preferentially, in axes coaxial to one another. Module pairs 102, 106, 107 may also be moved coaxially to one another so that the elongated medical members 103, 104, 105 are kept coaxial to one another in the telescopic form mentioned above.

The modules 102A, 102B may be moveable via a ball screw nut couplable to the guides within the linear drive 101. Additionally or alternatively, any other suitable method of moving the modules 102A, 102B axially may be used such as, for example, rack and pinion and/or rack and roller and/or belt gear and/or a piston and/or a pneumatic ram and/or an electromagnet.

As mentioned above, the modules 102A, 102B may be couplable to the linear drive 101. Every module is, preferably, made from two parts: a non-sterile module part, couplable with the linear drive 101, and a sterile part, which in this application is referred to as a cassette. Each module 102A, 102B may be identical to one another with respect to the non-sterile part, but the sterile parts of each module may be changeable according to the position of the module 102A, 102B with respect to the other module 102A, 102B and the elongated medical members 103, 104, 105 used (whether they be active, passive, bigger or smaller). The non-sterile parts may not be interchangeable, but the sterile parts may be interchangeable based on the use of the apparatus 100 and the elongated medical members 103, 104, 105 being used by the apparatus 100 and modules 102A, 102B. Preferably, the first nor the second motor are located within the sterile part of the modules 102A, 102B, but are located within the non-sterile part, and the non-sterile part is couplable with the linear drive 101.

In some examples, the gripping unit is located within the sterile part and/or the cassette. This may mean that only the sterile part of the modules 102A, 102B contact the elongated medical members 103, 104, 105, thereby ensuring that the elongated medical members 103, 104, 105 are kept sterile. In some examples, the non-sterile parts of the modules 102A, 102B have a sterile cover on them. The sterile cover may be removeable and replaceable to keep the apparatus 100 sterile. Other parts of the apparatus 100, such as the linear drive 101, may also comprise this sterile cover.

In this example, the cassette rotates together with the gripped elongated medical members 103, 104, 105 while non-sterile part of the module does not rotate and comprises only the linear movement. The cassette can be removed without removing the elongated medical members 103, 104, 105 from the patient. However, the rotational drive mechanism and the housing, cannot be removed without removing the elongated medical members 103, 104, 105 from the patient.

The gripping unit, and the gripper, may be activated in different ways. In the present example, the gripper is actuated via a compressed air mechanism. In some examples, an electrical motor may alternatively or additionally be used to actuate the gripper. In order to rotate the elongated medical members 103, 104, 105, the cassette rotates inside the non-rotatable module 102A, 102B. This may mean that the cassette rotates while the module 102A, 102B does not. The rotational motor in the module 102A, 102B may allow for the cassette to rotate, via a belt mechanism. Additionally or alternatively, a gear mechanism may be used to aid the rotation of the cassette, and therefore the elongated medical members 103, 104, 105. The cassette may be cylindrical, or in the form of a hexagonal prism, or in any other suitable form. A hexagonal prism may ensure that the cassette does not slip with respect to the receptacle of the non-sterile part that accepts the receptacle. The module 102A, 102B may also comprise a slipring for air transfer and/or a rotational slipring for communication/electrical signal transfer from the stationary non-sterile part to the rotational sterile part.

In the present example, the apparatus 100 is mounted on its own support, i.e. is freestanding, and attachable with a surgical table. However, in some examples, the apparatus may be smaller, weigh less and may be mountable on a surgical table. The mounting may be via screws, nut and bolts, magnets, or any other attaching means.

In some examples, the apparatus further comprises an anti-buckling device coupled between each module 102A, 102B of a module pair 102, 106, 107 and/or between modules 102A, 102B, 106A, 106B or different module pairs 102, 106, 107. The elongated medical member 103, 104, 105 may be fed through the center of the anti-buckling device. This may prevent buckling of the elongated medical members 103, 104, 105 during movement of the elongated medical members 103, 104, 105 and/or the modules 102, 106, 107, thereby improving the safety of the apparatus 100. An “accordion” style anti-buckling system is preferable. However, the system may additionally or alternatively comprise guides, actuatable parts, or any other means that allows for the reduction of buckling of the elongated medical members 103, 104, 105.

FIG. 2 shows a schematic view of an apparatus for controlling movement of a plurality of elongated members according to some example implementations as described herein.

The apparatus 200 of FIG. 2 is the same as the apparatus 100 of FIG. 1. Additionally, the modules 202A, 202B and module pairs 102 are, preferably, the same as the modules 102A and 102B and module pairs 102 as mentioned above in relation to FIG. 1, and numbering for these features shall be interchangeable. In this example, the second module 202B is “recentering”. This takes place when the second module 202B has advanced towards the first module 202A, and can no longer move towards the first module 202A. The gripper of the first module 202A grips the elongated medical member 103, 104, 105 and then the gripper of the second module ungrips the elongated medical member 103, 104, 105. The second module 202B then travels away from the first module 202A to a predetermined point and then regrips the elongated medical member 103, 104, 105. The first module 202A then ungrips the elongated medical member 103, 104, 105 and the process as described with relation to FIG. 1 to advance, retract and rotate the elongated medical member 103, 104, 105 can take place. This process can also happen in the reverse when the second module 202B has travelled too far from the first module 202A of the module pair 202.

In the case of there being a plurality of module pairs 102, 106, 107, the movement of FIG. 1 and the recentering of FIG. 2 may takes place in the following fashion:

When the second module 102B of a first module pair 102 grips the elongated medical member 103, 104, 105 within its gripper, it may then move to advance and retract the elongated medical member 103, 104, 105 towards or away from the patient, and may also rotate the elongated medical member 103, 104, 105. As the second module 102B of the first pair 102 moves towards the first module 102A of the first pair 102, a first module 106A of a second pair 106 may move in conjunction with the second module 102B of the first pair 102. This may allow for the elongated medical member 103, 104, 105 to be sufficiently supported, thereby reducing the flexure of the elongated medical member 103, 104, 105 and elongating the lifespan of the elongated medical member 103, 104, 105. Once again, this process may happen in the reverse should the second module 102B travel too far from the first module 102A of the module pair 102.

As the second module 102B of the first pair 102 recenters, as seen in FIG. 2, a second module 106B of the second pair 106 may move in conjunction with the second module 102B of the first pair 102. This may allow for the elongated medical member 103, 104, 105 to be sufficiently supported, thereby reducing the flexure of the elongated medical member 103, 104, 105 and elongating the lifespan of the elongated medical member 103, 104, 105.

Although first and second modules 102A, 102B, 106A, 106B in the first and second module pairs 102, 106 are mentioned above, it is to be understood that the second hand 106B of the second pair 106 may move during the advancement and retraction of the elongated medical member 103, 104, 105 and/or the first hand 106A of the second pair 106 may move during the recentering process.

At least one of the modules 106A, 106B of the second module pair 106, and preferably the first module 106A during advancement/retraction of the elongated medical member 103, 104, 105, may move in a manner 1:1 with the movement of the second module 102B of the first pair 102, or may have a movement scale factor applied to said movement, in order to allow the elongated medical member 103, 104, 105 to be sufficiently supported.

The modules 102A, 102B may comprise at least one of the following features and/or abilities:
1. A sterile cassette, where the sensor and/or the gripper is integrated into said sterile cassette;
2. Each module pair 102, 106, 107 is configured to manipulate one elongated medical member 103, 104, 105, so in a system any number of elongated medical members 103, 104, 105 may be simultaneously manipulated; and

The apparatus 100 may comprise at least one of the following features and/or abilities:
1. The apparatus 100 may comprise with more than one, preferably three, module pairs 102, 106, 107 for remote manipulation of a corresponding number of elongated medical members 103, 104, 105;
2. The apparatus with more than one module pair 102, 106, 107 for remote manipulation of three elongated medical members 103, 104, 105 simultaneously, wherein the elongated medical members 103, 104, 105 have unlimited rotational and linear movement;
3. An apparatus 100 for remote manipulation of three OTW (over the wire) type elongated medical members 103, 104, 105 simultaneously; and
4. A manipulation principle, so that a module 102A, 102B can grip the elongated medical member 103, 104, 105, sense the elongated medical member 103, 104, 105, move the elongated medical member 103, 104, 105 in linear direction, and rotate the elongated medical members 103, 104, 105.

FIGS. 3 and 4 show schematic views of a human control unit for manipulating a remote elongated medical member according to some example implementations as described herein.

In particular, FIGS. 3 and 4 show a master apparatus 300, 310 that may be used in conjunction with the slave apparatus 110 described above. The master apparatus 300, 310 may be located in the same room or location as the slave apparatus 110, or may be located remote from the slave apparatus 110 in a different room of the same building, or a different location altogether.

Furthermore, the master apparatus 300, 310 allows for remote manipulation of an elongated medical member and, in particular, an endovascular elongated medical member, although any type of elongated medical member can additionally, or alternatively, be used.

The master apparatus 300, 310 comprises a linear drive 301 similar to the linear drive 101 mentioned above in connection with FIGS. 1 and 2.

In this example, the master apparatus 300, 310 also has three modules 302, 306, 307 to cooperate with the three module pairs 102, 106, 107 of FIGS. 1 and 2, with each module 302, 306, 307 corresponding to a module pair 102, 106, 107 of the slave apparatus 110. At least one of the modules 302, 306, 307 comprises a gripping unit configured to grip an elongated member. In some examples, the gripping unit may ungrip the elongated medical member 303, 304, 305 for repair and/or cleaning purposes, but grip the elongated medical member 303, 304, 305 during use of the master apparatus 300, 310. This gripping unit may be the same, or similar to, the gripping unit described above in relation to FIGS. 1 and 2 and/or the gripper described below. In a preferable example, the elongated medical instruments 103, 104, 105 at the slave apparatus 110 are the same as the elongated members 303, 304, 305 at the master apparatus 300, 310. That is to say, if a guidewire 103 300 cm in length, 0.14 inches in diameter and made of a soft, flexible material suitable for insertion into a patient is used at the slave apparatus 110, an exact replica of the guidewire 303 may be used at the master apparatus 300, 310. In an alternate example, the guidewire 303 at the master apparatus 300, 310 may only be 100 cm in length, 0.38 inches in diameter and made of a stiff material. In other words, corresponding elongated members 103, 104, 105, 303, 304, 305 at the slave 110 and master 300, 310 apparatuses may be identical, or may have different physical properties, such as, for example, stiffness, length, diameter and type of elongated member and/or different chemical properties such as, for example, stiffening materials, hydrophobic coatings and sterile coatings. The same principles apply to the other two elongated members 304, 305 shown in FIG. 3 and the other two elongated medical members 104, 105 shown in FIGS. 1 and 2. This may allow for the surgeon, or user, at the master apparatus 300, 310 to receive realistic haptic feedback, as will be described below. In the example shown here, three elongated members 303, 304, 305 are mentioned, but it is to be understood that there may be any number of elongated members 303, 304, 305 at the master apparatus 300, 310. In a preferred example, the number of elongated members 303, 304, 305 at the master apparatus 300, 310 and the number of elongated medical members 103, 104, 105 at the slave apparatus 110 are the same, and each master apparatus member 303, 304, 305 is linked to one slave apparatus member 103, 104, 105. In some examples, there may be fewer master apparatus members 303, 304, 305, and one master apparatus member 303, 304, 305 is configured to manipulate a plurality of slave apparatus members 103, 104, 105. In some examples, there may be fewer slave apparatus members 103, 104, 105, and a plurality of master apparatus members 303, 304, 305 are configured to manipulate one slave apparatus member 103, 104, 105. In some examples, the system may comprise one master apparatus member 303, 304, 305 configured to manipulate a plurality of slave apparatus members 103, 104, 105 and a plurality of master apparatus members 303, 304, 305 being configured to manipulate one slave apparatus member 103, 104, 105.

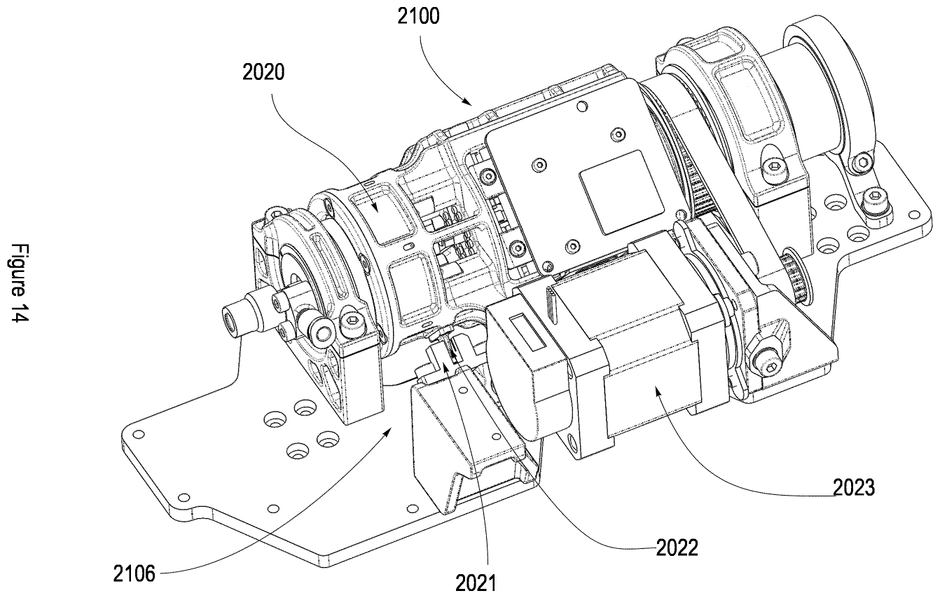

The haptic feedback may be generated by a sensor comprised in at least one of the modules 302, 306, 307 of the master apparatus 300, 310. In some examples, the sensor is comprised in a haptic feedback unit/module. The sensor may be the same, or similar to, the sensor described above in relation to FIGS. 1 and 2 and/or the sensor described in relation to FIGS. 13 to 22. The sensor may additionally or alternatively receive a control signal from the slave apparatus 110 indicating the status of at least one of the elongated medical devices 103, 104, 105 at the slave apparatus 110. The sensor of the module may measure the difference in axial and/or rotational force between the received control signal, and the axial and/or rotational force being applied by the user at the master apparatus 300, 310 and advance or retract the module 302, 306, 307 and/or rotate the elongated member 303, 304, 305 in order to balance the measured forces between the slave apparatus 110 and the master apparatus 300, 310. That is to say, the modules 302, 306, 307 of the master apparatus 300, 310 may have the same four modes of operation as described above in relation to the slave apparatus 110. In some examples, at least one of the modules 302, 306, 307 has at least one of the aforementioned modes of operation. The above may allow for the user of the master apparatus 300, 310 to receive realistic haptic feedback from the slave apparatus 110, may allow for the user to accurately feel what is happening at the slave apparatus 110, and may allow for the user to receive the same haptic feedback as they would if they were bedside and manually manipulating the elongated medical members 103, 104, 105. In a similar fashion, the sensor may send a control signal to the slave apparatus 110, and the sensor at the slave apparatus 110 may perform a similar force balancing, as described above, in order to manipulate at least one of the elongated medical members 103, 104, 105 at the slave apparatus 110. The control signals may be transmitted and/or received by a connection 309 with the slave apparatus 110. This connection 309 may be wired and/or wireless. In some examples, information gathered from the sensors may be transmitted to the slave apparatus 110 via this connection 309. Additionally or alternatively, the apparatus 300, 310 may receive instructions from the slave apparatus 110 via this connection 309.

In the above, as an example, the user side, i.e. the side of the human control unit 300, 310, the user may start to move the local, elongated medical member 303, 304, 305. The local elongated member 303, 304, 305 is preferably fixed with respect to its respective gripping unit 302, 306, 307, and the gripping unit 302, 306, 307 is coupled to a spring and/or a linear drive and/or a rotational drive (not shown) to allow for the movement and rotation of the gripping unit 302, 306, 307. To move the local elongated member 303, 304, 305, the user exerts a force sufficient to overcome the biasing force of the spring and move the local elongated member 303, 304, 305 a particular distance. For example, a 1 N force may cause a 1 mm movement until the force between the spring and the user's exerted force become equal. At the same time, on the patient side 100, 110, 200 of the system, the remote elongated medical member 103, 104, 105 may be also moved 1 mm, and stops moving when the forces on the user side equalize.

As an additional example, a continuous, unbalanced force of 1 N is assumed at the user side 100, 110, 200. In this example, the local elongated medical member 303, 304, 305 is moved in a similar manner as described above, but encounters a blockage and so, the readings of the patient side sensor (see sensor described below) becomes 1 N. This may indicate to the user that there is a blockage at the patient side 100, 110, 200 of the system. If the user keeps increases the exerted force to, for example, 3N, the elongated members 103, 104, 105, 303, 304, 305 of the user 300, 310 and patient 100, 110, 200 sides translate until the forces balance, so until the force on the patent side 10, 110, 200 also reaches 3 N.

At this moment, if the user releases the local elongated medical member 303, 304, 305, due to the spring coupled to the gripping unit 302, 306, 307, the local elongated medical member 303, 304, 305 moves back at its zero point with a balanced force of ON, i.e. its predetermined zero point. Resultantly, the remote elongated medical member 103, 104, 105 also moves its predetermined zero point. This is to say that the user tries to exert a force on the local elongated medical member 303, 304, 305 and feels the resistive force from the spring. If a dead man's switch is used, as described in the present application, the system stays in 3 N=3 N situation when the switch is in a first position, and when the switch is moved to the second position, the first elongated medical member and the second and/or third elongated medical member would revert to their respective zero points. In some examples, if the dead man's switch is in the first position holding the elongated medical instruments in place, this switch could be overcome by the user exerting more force on the local elongated medical member 303, 304, 305 if the user wants to take the control of the member 303, 304, 305 from the system. This additional force may be altered depending on the situation the system is used in and/or user preference.

Additionally or alternatively, although the above example is described in relation to force being exerted on the local elongated medical member 303, 304, 305 on the user side 300, 310, the same principles apply to a force being applied to the remote elongated medical member 103, 104, 105 on the patient side 100, 110.

In a preferred example, there is a system comprising the master apparatus 300, 310 and at least one slave apparatus 110. The slave apparatus 110 is preferably for manipulation of elongated medical members 103, 104, 105 at a surgical area, in an area affected by X-rays, near a patient. Alternatively, the area may not be a surgical area, but an area where a patient is undergoing a non-surgical procedure. Additionally or alternatively, the area may be affected by an MRI machine and/or may be under the surveillance of a camera and/or any other suitable observation method. The master apparatus 300, 310 is for manipulation of elongated members 303, 304, 305 at remote location, wherein the remote location is a different location from the location where the slave apparatus 110 is located. The slave apparatus 110 and the master apparatus 300, 310 may be coupled via a wired connection, such as, for example, LAN and/or Internet/Ethernet cable and/or any other suitable wired means, and/or via wireless means such as, for example, Bluetooth and/or Wi-Fi and/or satellite, and/or any other suitable wireless means. The system combining the slave 110 and master 300, 310 apparatuses may allow a manipulation of an elongated member 303, 304, 305 at the master apparatus 300 to be translated into the into a module pair 102, 106, 107, and therefore a remote manipulation of an elongated medical member 103, 104, 105 is produced in line with a user's manipulation of the elongated member 303, 304, 305 at the master apparatus 300. This may allow for a user of the master apparatus 300, 310 to use two fingers to receive haptic feedback and to manipulate the elongated member 303, 304, 305. Usually, a surgeon/user, in manual manipulation, is able to manipulate up to two instruments by themselves, and three elongated medical members with the aid of a second user, such as a nurse. The present system allows for more than two elongated medical members by a single person, thereby improving the efficiency of the manipulation of said members, reducing the number of people needed during a manipulation of the elongated medical members, and increasing the safety of an operation as communication is not needed between multiple people, thereby reducing the chance of miscommunications.

The system allows for the simultaneous manipulation of up to three elongated medical members, in the present example, wherein the elongated medical members are telescopically arranged one inside the other. It may also allow for simultaneous haptic/tactile feedback being received from all three elongated medical members in the direction from the slave apparatus 110 (surgical location) to the master apparatus 300, 310 (remote location) and vice versa. As mentioned above, although three elongated medical members are mentioned, it is to be understood that there may be any number of elongated medical members at the slave apparatus 110 and any number of elongated members at the master apparatus 300, 310. The system, preferably, keeps the axial and/or rotational forces balanced at the slave 110 and master 300, 310 apparatuses via the haptic feedback unit/module described above. In some examples, the forces are balanced between a single module pair 102, 106, 107 at the slave apparatus 110 configured to manipulate at least one of the elongated medical members 103, 104, 105, and a corresponding module 302, 306, 307 and elongated member 303, 304, 305 at the master apparatus 300, 310. For example, a user manipulates a first elongated member 303, 304, 305 at the master apparatus 300, 310 and resultantly, by measuring the axial and/or rotational forces caused by the manipulation, and by measuring the axial and/or rotational forces being experienced by the corresponding elongated medical member 103, 104, 105 at the slave apparatus 110, and balancing the resultant forces, the first elongated medical member 103, 104, 105 at the slave apparatus 110 can be remotely manipulated in the same way as the elongated member 303, 304, 305 at the master apparatus 300, 310 was manipulated by the user. If there is an obstacle in the way of the elongated medical member 103, 104, 150 at the slave apparatus 110, the slave apparatus 110, via the sensor, senses this obstruction and, via the force balancing principle working in the opposite direction from that described above, haptic feedback is provided to the haptic feedback module/unit of the master apparatus 300, 310 and the elongated member 303, 304, 305 at the master apparatus 300, 310 is manipulated in a similar way.

In a preferred example, as mentioned above, the user of the master apparatus 300, 310 has a replica of the elongated medical members 103, 104, 105 of the slave apparatus 110 in front of them, such as, for example, a catheter and a guidewire. This may allow for the surgeon to have a more realistic feel of the surgery that is taking place. In some examples, at least one of the modules 102A, 102B at the slave apparatus 110 and/or at least one of the elongated medical members 103, 104, 105 at the slave apparatus 110 may comprise a sensor configured to sense the resistive force encountered by the elongated medical members 103, 104 105, in some examples, inside a patient. This force may then be transmitted to the master apparatus 300, 310 to give the user a realistic feel of the forces being experienced by the sensor, even if the slave 110 and master 300, 310 are in different locations. However, this user interface is not limited to a replica of the elongated medical members at the slave apparatus 110, but may be generic replacements that still allow for the user to receive the haptic feedback from the slave apparatus 110.

In some examples, a scale factor may be applied between movements of the elongated members 303, 304, 305 at the master apparatus 300, 310 and the elongated medical members 103, 104, 105 at the slave apparatus 110. That is to say, that there may be a difference in the factor of the rotation and/or axial movements between the two apparatuses 110, 300, 310. In one example, there may be a 10× scale factor in relation to the axial movement. Resultantly, in order to create an axial movement of 10 mm at the slave apparatus 110, the corresponding elongated member 303, 304, 305 at the master apparatus 300, 310 would need to be axially moved 100 mm. This may allow for the precision of the manipulation of the members 103, 104, 105 at the slave apparatus 110 to be improved while maintaining the haptic feedback characteristics of the system. the above principle may be applied to the axial and/or rotational movements, and/or for only select elongated medical instruments 103, 104, 105.

The master apparatus 300, 310, as mentioned above, has, preferably, a user interface of such a type so that the interface is similar to real use of elongated endovascular instruments. The interface preferably has the elongated members 303, 304, 305 placed in comfortable position for the user. In some examples, the initial position of at least one of the elongated members 303, 304, 305 is adjustable depending on the user's preference. The elongated members 303, 304, 305 can be the same as, or different than, the elongated medical members 103, 104, 105 of the slave apparatus 110. In some examples, the elongated members 303, 304, 305 of the master apparatus 300, 310 might be cut and/or mounted one inside the other to make them easier to use by the user.

Near the user interface, a touchscreen display may be provided in such way to allow the user to, while manipulating of the elongated members 303, 304, 305, to initiate stop/freeze of a particular elongated member position by touching the touchscreen. The touchscreen comprises, in this example, three sections, wherein the first section 311 corresponds to a first elongated member 303, a second section 312 corresponds to a second elongated member 304 and a third section 313 corresponds to a third elongated member 305. The touchscreen is preferably located on a surface, such as a table 314. The movement of each of the elongated members 303, 304, 305 is indicated by the arrows 321, 322, 323 of FIG. 4. It is to be understood that the number of sections 311, 312, 313 may change depending on the number of elongated members 303, 304, 305 at the master apparatus 300, 310. Having such stop/freeze buttons, as mentioned above, may allow for the user to safely, and easily, manipulate any number of elongated members 303, 304, 305 simultaneously. As an example, if a user wants to keep the second instrument 304 at a stable position, the user can press the second section 312 of the touchscreen and continue manipulating only the first 303 and third 305 elongated members. Such a stop/freeze feature may be implemented in such a way that it physically stops the particular instrument tip, at the slave apparatus 110, at its position when the second section 312 of the touchscreen was pressed, or it may virtually stop the particular position of paired instrument tip at the slave apparatus 110, so that each of the other modules 102A, 102B, 302 at the slave 110 and master 300, 310 modules to move while maintaining the frozen/stopped instrument end tip in a steady position.

The stopping/freezing feature may be realized in two ways. The first is that the master apparatus 300, 310 stops transmitting data relating to the state of the frozen/stopped elongated member 303, 304, 305 to the slave apparatus 110. This may mean that the elongated member 303, 304, 305 is still rotatable and translatable, but this information is not transmitted. The second way is that the module 302, 306, 307, associated with the frozen/stopped elongated member 303, 304, 305 is frozen in place, thereby not allowing the frozen/stopped elongated member 303, 304, 305 to be manipulated in any way. In some examples, data regarding the frozen/stopped elongated member 303, 304, 305 is not transmitted and the module 302, 306, 307 is frozen in place.

At least one of the sections 311, 312, 313 of the touchscreen may comprise information on a state of an elongated member 303, 304, 305 associated with said section 311, 312, 313. The state of the local elongated member may relate to a distance in a predetermined direction the elongated member 303, 304, 305 has been moved from a zero point and/or a rotation angle of the elongated member 303, 304, 305 from a predetermined zero point and/or a force being exerted on the elongated member 303, 304, 305 and/or a rate of change of speed and/or angle of the elongated member 303, 304, 305 and/or a name of the elongated member 303, 304, 305 and/or a diameter of the of the elongated member 303, 304, 305, as it is being rotated by the user and/or any other suitable state that can be displayed to the user.

Additionally or alternatively, the same, or different, states of the elongated member 103, 104, 105 of the slave apparatus 110 can be displayed to the user. The axial translation can be measured, for example, by measuring an axial movement of the module 302 at the master apparatus 300, 310 and/or a movement of a module 102A, 102B at the slave apparatus 110. The same principle applies for the rotational movement. In some examples, should a total movement and/or a rate of change of movement approach a predetermined limit, a visual and/or audio and/or haptic warning may be emitted by the touchscreen, or a speaker, or any other suitable device, indicating to the user that a limit is about to be breached. This may allow for the lifetime of the system to be extended as members 103, 104, 105, 303, 304, 305 are not used beyond their limits, thereby reducing the stresses exerted on said members 103, 104, 105, 303, 304, 305.

Additionally, in some examples, each of the elongated members 303, 304, 305 may be controlled individually. For example, the outer guidewire 303 may be “frozen” in place, i.e. not moveable, as described above, and only the inner catheter 304, 305 may be moved due to the manipulation mentioned in the present writ. Alternatively, the inner catheter 304, 305 may be “frozen” and the outer guidewire 303 may be manipulatable.

The length of the sections 311, 312, 313 of the touchscreen may alter based on the length of the elongated member 303, 304, 305 available to be manipulated by the user. This is to say that when the user manipulates the elongated member 303, 304, 305, stop/freeze button will always stay nearby. As an example, in the case of two elongated members 303, 304, 305, in an initial position, the first member 303 may take up 50% of the user interface and the second member 304 may take up the other 50%. Resultantly, the first 311 and second 312 section of the touchscreen are split 50/50 between the two members 303, 304. Should the first member 303 be retracted, so that said member 303 takes up 70% of the interface, the first section 311 of the touchscreen automatically adjusts accordingly, and will take up 70% of the touchscreen. Consequently, during this movement of the first member 303, the area of the touchscreen taken up by the second section 312 gradually reduces to 30% of the area of the touchscreen. This same principle is applicable to any number of elongated members 303, 304, 305 and any number of sections 311, 312, 313 of touchscreen.

Although a touchscreen is mentioned above, at least one section 311, 312, 313 of the touchscreen may be replaced, or supplemented, with at least one of any other suitable type of button, lever, trigger, position sensor, position tracker, camera vision or any other suitable mechanism that allows for the user to freeze the position of an elongated member 303, 304, 305 located at the master apparatus 300, 310.

The master apparatus 300 may comprise at least one of the following features:
1. The described principle of the user interface to provide haptic feedback;
2. Elongated member 303, 304, 305 stop/freeze to keep a tip of said member 303, 304, 305 in a fixed position while simultaneously allowing the manipulation of other elongated members 303, 304, 305;
3. Elongated member 303, 304, 305 stop/freeze to keep a tip of said member 303, 304, 305 in a fixed position while simultaneously allowing the manipulation of at least one module 302, 306, 307; and
4. A touchscreen, where sections 311, 312, 313 of said touchscreen are altered dependent on a state of an elongated member 303, 304, 305 assigned to said section 311, 312, 313.

FIG. 5 shows a perspective view of a schematic illustration of the gripper according to some example implementations as described herein.

The gripper 1000 comprises a guide 1001, a first gripping component 1002, a second gripping component 1003 and a plurality of assembly pins 1004.

The guide 1001 is configured to guide at least the second gripping component 1003, as will be described in more detail below. In this example, there are a plurality of assembly pins 1004, but there may be a single assembly pin 1004 or no assembly pins 1004. The number of assembly pins 1004 may vary depending on the design of the gripper 1000. In some examples, the assembly pins 1004 are configured to couple the first gripping component 1002 to the guide 1001, thereby ensuring that the first gripping component is stationary with respect to the guide 1001. In some examples, the assembly pins 1004 are configured to couple the first gripping component 1002 and/or the guide 1001 to an external component (not shown). The external component may be a sensor, a housing, an actuator, a part of an endovascular system or any other suitable component.

The guide 1001 further comprises a first portion 1010 and a second portion 1011. The first portion 1010 is configured to guide at least a portion of the second gripping component 1002. The guide 1001 may also guide an elongated member (see FIG. 3). The second portion 1011 of the guide 1001 is discrete from the first portion 1010 and extends outwardly from the first portion 1010 of the guide 1001. The second portion 1011 may also be configured to guide at least a portion of the elongated member, wherein the first 1010 and second 1011 portions of the guide 1001 guide different portions of the elongated member. The first 1010 and second 1011 portions of the guide 1001 are not limited to the design shown in FIG. 1 but may be of any suitable design which allows for the guiding of the elongated member and, in the case of the first portion, the guiding of at least the second griping component 1003.

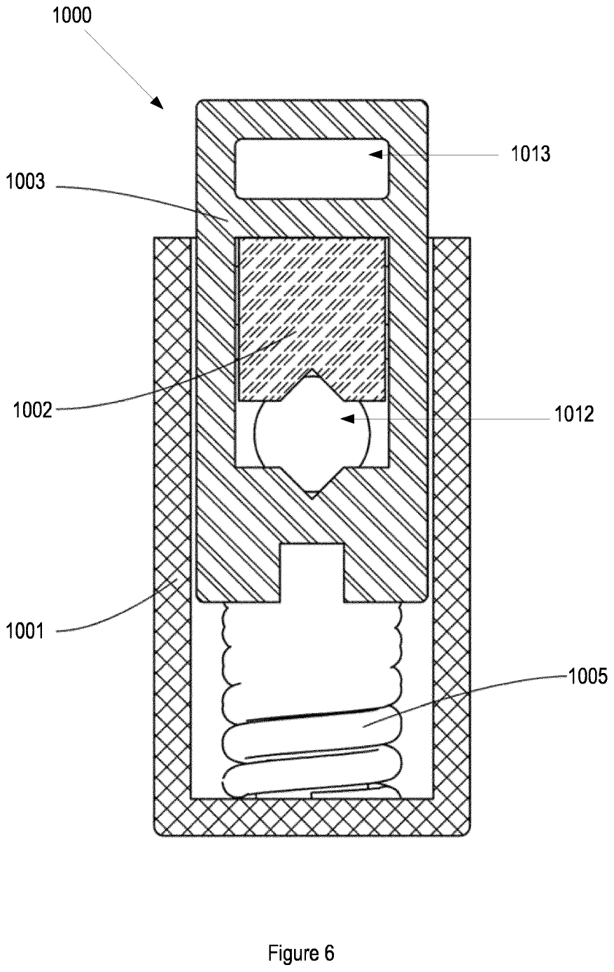

FIG. 6 shows a cross-sectional view of a schematic illustration of parts of the gripper according to some example implementations as described herein.

In FIG. 6, it can be seen that the second gripping component 1003 is coupled to a resilient member 1005, in this case a spring. The resilient member 1005 may alternatively be any suitable component which provides a resilient force. The guide further comprises a through hole 1012 configured to accommodate the elongated member and allow for the elongated member to travel through the gripper 1000. The second gripping component 1003 also comprises an opening 1013 which extends beyond the guide 1001. The opening 1013 may allow for a weight reduction of the gripper 1000. This may, in turn, result in an increase in performance as high inertia may reduce the performance of the gripper 1000.

Furthermore, in this example, the guide 1001 is configured to surround the spring 1005, the first gripping component 1002 and at least a portion of the second gripping component 1003. This may allow for parts of the gripper 1000 to be guided in a manner which reduces unwanted stress and shear forces during operation of the gripper 1000, thereby extending the lifetime of the gripper 1000. In some examples, the guide 1001 guides only the second gripping component 1003 and/or the spring 1005.

The spring 1005 coupled to the second gripping component allows for the second gripping component to be moveable with respect to the guide 1001. The resilient force provided by the spring 1005 preferably biases a first surface of the second gripping component 1003 towards a first surface of the first gripping component 1002, wherein the biasing results in the elongated member being gripped between the first 1002 and second 1003 gripping components, as will be described in more detail below. In some examples, a force provided by a pneumatic cylinder and/or an electric motor and/or any other suitable component against the resilient force provided by the spring 1005 allows for the first surface of the second gripping component 1003 and the first surface of the first gripping component 1002 to be moved away from each other, thereby allowing for the elongated member to be ungripped (released) and allow for said elongated member to be moved within the gripper 1000 or removed from the gripper 1000 entirely. This may also allow for an elongated member to be inserted into the gripper 1000.

The through hole 1012 is of a size and dimension which allows for the elongated member to be guided through the guide 1001 and also allows for the elongated member to be inserted and/or removed from the guide 1001. In some examples, there is only one hole in the guide 1001, i.e. the elongated member cannot extend through the guide 1001.

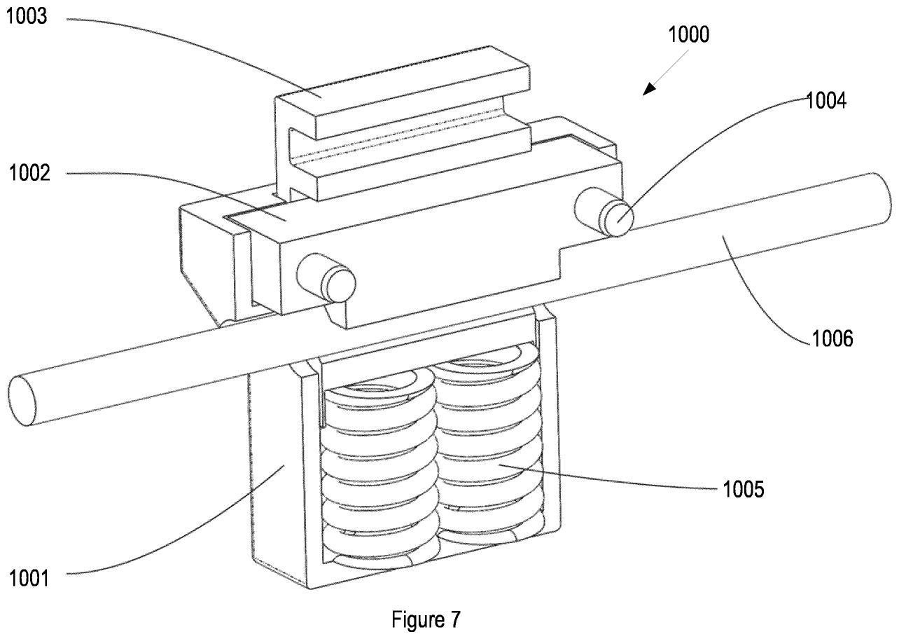

FIG. 7 shows a cut-away view of a schematic illustration of the gripper according to some example implementations as described herein.

It can be seen in FIG. 7 that the elongated member 1006 extends though the guide 1001 of the gripper via the through holes 1012 mentioned above. This may allow for the elongated member 1006 to be of a length substantially longer than the distance between the through holes 1012 in the guide 1001. This may be particularly advantageous in some scenarios. In particular, if the elongated member 1006 is moved through the holes 1012 via a “shuffling” technique where the elongated member 1006 is fed through the holes 1012 in gradual steps, the elongated member 1006 being longer than the distance between the holes 1012 may allow for the elongated member to be fed in gradual steps through the said holes 1012 to achieve this technique.

It can be further seen in FIG. 7 that the assembly pins 1004 are located within the first gripping component 1002 and that these assembly pins 1004 will allow for the first gripping component 1002 to be coupled to the guide 1001. In this example, the assembly pins 1004 are pins but they may additionally or alternatively be a nut and bolt, a screw, a hinge, a bayonet coupling, a welding or any other suitable type of coupling or any combination thereof.

The elongated member 1006 may be any elongated member which is preferably suitable for endovascular purposes, in particular a catheter or a guidewire. The elongated member 1006 is not limited to the endovascular purposes and may alternatively be any type of elongated member which requires to be gripped.

Furthermore, in this example, there are two springs 1005 coupled to the second gripping component 1003. There may be a single spring 1005 or any number of springs 1005 which allow for the functioning of the gripper 1000 as described in the present disclosure.

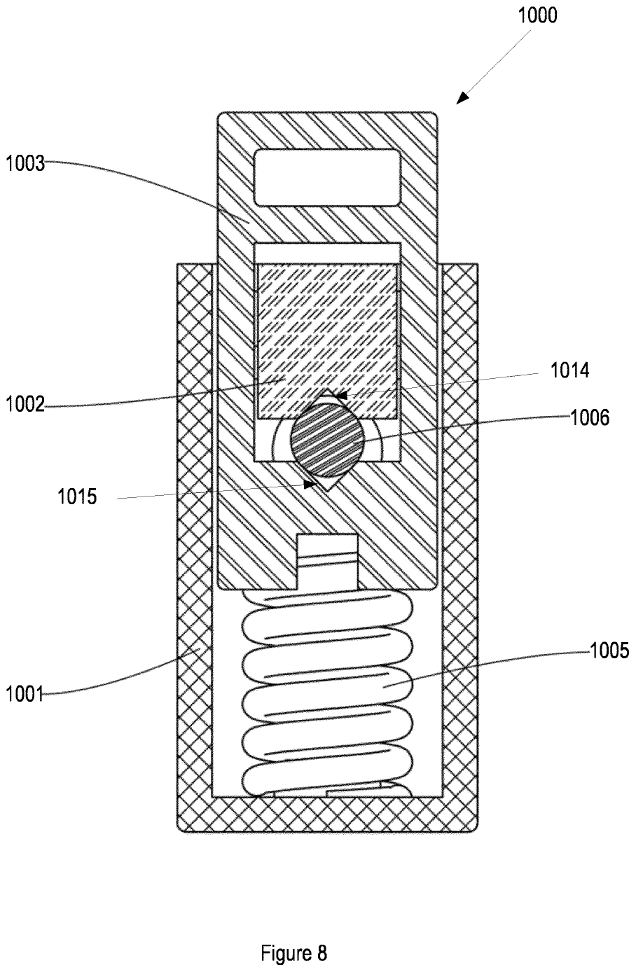

FIG. 8 shows a cross-sectional view of a schematic illustration of parts of the gripper according to some example implementations as described herein.

In this example, the first gripping component 1002 and second gripping component 1003 each comprises a respective recess 1014, 1015. The recesses are configured to accommodate at least a portion of the elongated member 1006 within the gripper 1000. In this example, both of the recesses 1014, 1015 are V-shaped but they may alternatively be any suitable shape such as, for example, cuboidal, semi-cylindrical or a bespoke shape.

In some examples, the first gripping component 1002 comprises a metallic portion, wherein the metallic portion is configured to contact at least a portion of the elongated member 1002. The metallic portion preferably comprises aluminum, and in particular 7075-t6 aluminum. The elongated member preferably comprises a plastic or polymer contactable with the metallic portion. This in turn may provide for a particularly strong gripping of the elongated member 1006, thereby reducing the likelihood of the elongated member 1006 moving while being gripped by the gripper 1000.

In this example, the first gripping component 1002 is partially housed by the second gripping component 1003. This may allow for the movement of the second gripping component 1003 to be limited as it may contact the first gripping component 1002. This may result in a reduction of unwanted forces being exerted on the second gripping component 1003, thereby extending the lifespan of the second gripping component 1003.

The second gripping component 1003 is configured to be slideable past both the guide 1001 and the first gripping component 1002. This may allow for the second gripping component 1003 to be limited to moving in a single axis, i.e. the axis of the exertion of the biasing force, thereby reducing unwanted movement by the second gripping component 1003 during movement of the second gripping component 1003 and while the elongated member 1006 is being gripped. This may also result in a reduction of unwanted forces being exerted on the elongated member 1006, thereby also increasing the lifespan of the elongated member 1006.

In FIG. 8, the gripper 1000 is in the gripping position, i.e. the elongated member 1006 is being gripped between the first 1002 and second 1003 gripping components. As described above, the spring 1005 provides a biasing force which biases the second gripping component 1003 towards the first gripping component 1002, thereby keeping the elongated member 1006 gripped. In some examples, a force exerted by a pneumatic cylinder and/or an electric motor and/or any other suitable component against the biasing force allows for the elongated member 1006 to be moved, removed or inserted into the gripper. Once this force has been removed, the biasing force biases the second gripping component 1003 and the elongated member 1006 is once again gripped by the gripper 1000. This also means that the elongated member 1006 is gripped via a compressive force. The use of compressive forces may allow for a more secure gripping and/or securing of the elongated member 1006 and may allow for the lifetime of the elongated member 6 to be increased, as it is not subjected to shear or tension forces.

The V-shaped recesses 1014, 1015 allow for the elongated member 1006 to be contacted, along four contact lines, by said recesses 1014, 1015. The use of contact lines may allow for the compressive force to be exerted along the portion of the elongated member 1006 which is contacted by the recesses 1014, 1015, thereby reducing the force that is exerted on any single portion of the elongated member 1006. This may allow for the elongated member 1006 to have a longer lifespan, as the compressive force is not focused at a single point. It may also allow for a more secure gripping of the elongated member 1006, as the elongated member 1006 is contacted along a portion of said elongated member 1006 by the first 1002 and second 1003 gripping components, thereby increasing the area of contact where the elongated member 1006 is contacted by the gripping components 1002, 1003.

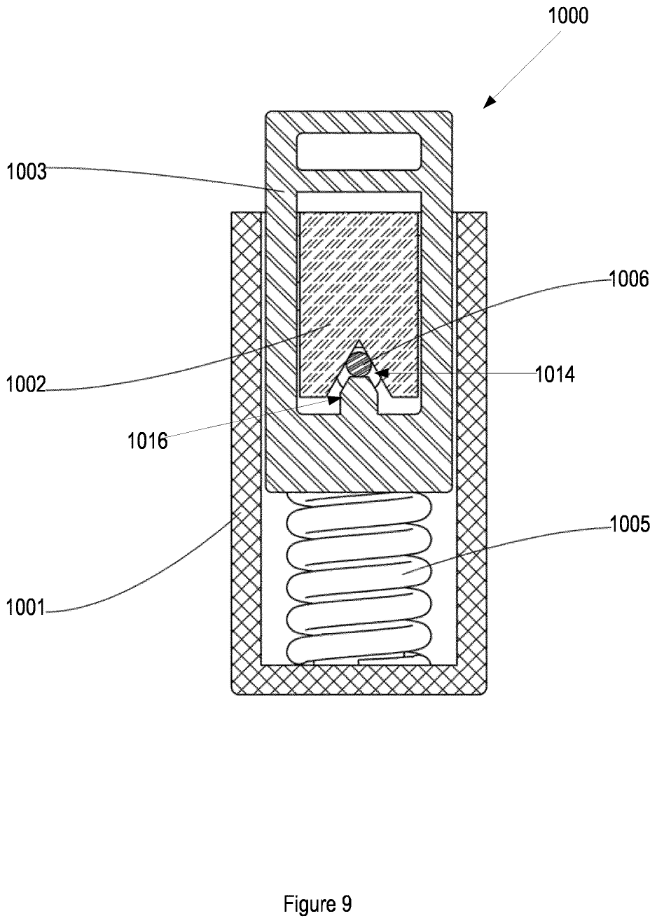

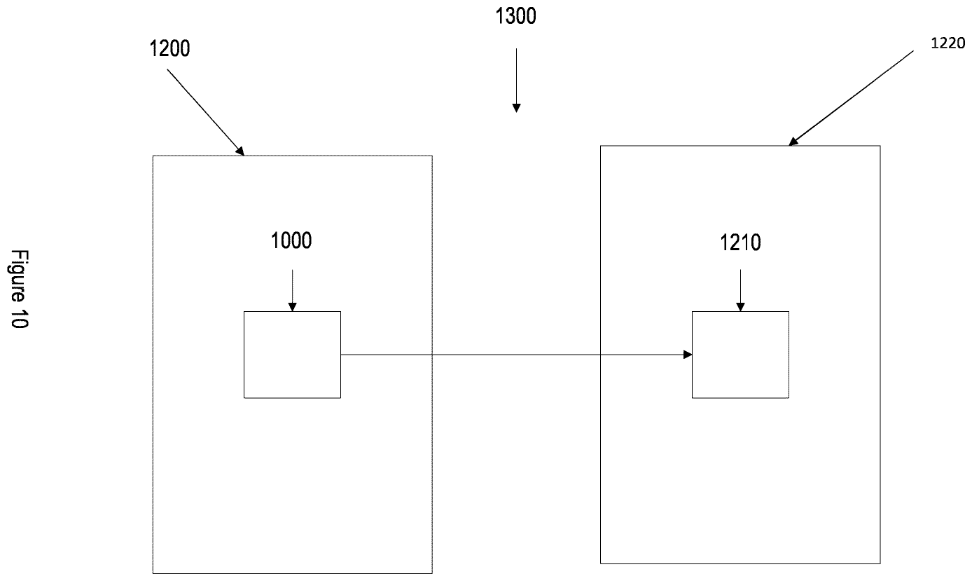

FIG. 9 shows a cross-sectional view of a schematic illustration of parts of the gripper according to some example implementations as described herein.

In this example, the first gripping component 1002 comprises a recess 1014, as described above, but the second gripping component alternatively comprises a protrusion 1016. The protrusion may be formed via 3D printing but may additionally or alternatively be produced by milling and/or electrical discharge machining (EDM). In this example, the protrusion 1016 comprises a truncated V-shape, wherein the truncated V-shape is configured to contact the elongated member 1006. The sloped faces of the truncated V-shape are preferably angled at the same angle as the faces of the V-shaped recess 1014. This may allow for a particularly secure gripping of the elongated member 6 when the elongated member 1006 is being gripped. The protrusion may additionally (at one or more other portions) or alternatively comprise other shapes such as, for example, cuboidal, semi cylindrical, prismoidal or a bespoke shape.

As can be seen in FIGS. 8 and 9, a design of the gripper 1000, and in particular the first 1002 and second 1003 gripping components may be altered depending on a parameter of the elongated member 1006. The parameter may be, for example, a material of the elongated member 1006, a dimension of the elongated member 1006, a usage of the elongated member 1006, a resilience of the elongated member 1006 or any other suitable parameter (or, in case more parameters are taken into account, any combination thereof).

Furthermore, the biasing force provided by the spring 1005 coupled to the second gripping component may be altered depending on one of these parameters. In a non-limiting example, if the elongated member 1006 is a guidewire, a biasing force of up to 5 Newton may be exerted by the spring 1005 on the second gripping component and if the elongated member 1006 is a catheter, the biasing force may be of up to 15 Newton. The biasing force may be altered via a replacement of the gripper 1000 with a second gripper, wherein the springs 1005 of the second gripper provide a biasing force which is suitable for the elongated member 1006. Additionally or alternatively, the gripper 1000 may further comprise a force sensor configured to sense a compressive force being exerted on the elongated member 1006. The force sensor may then indicate, via audio, visual or haptic means to the user that the compressive force is too large for the elongated member 1006. In some examples, the force sensor may automatically ungrip (release) the elongated member 1006, i.e. provide a force against the biasing force and/or stop the gripper 1000 from exerting more force onto the elongated member 6 by preventing a movement of the second gripping component 1003. A user may be able to indicate to the force sensor compressive force limit via an input display on the gripper 1000 and/or via a dial on the gripper 1000 and/or via any other suitable method. In some examples, the user may input a type of elongated member 1006, i.e. a catheter or a guidewire, and the force sensor may then automatically determine a compressive force limit based on a predetermined input to the force sensor.

FIG. 10 shows a schematic block diagram of an endovascular system according to some example implementations as described herein.

The endovascular system 1300 comprises a first 1200 and a second 1220 endovascular instrument. The first 1200 and second 1220 endovascular instruments may be located in discrete locations and a movement of the first endovascular instrument 1200 may be translated (mimicked) to a corresponding movement in the second endovascular instrument 1220. In particular, a movement of the gripper 1000 in the first endovascular instrument may be translated into a movement in an instrument 1210 in the second endovascular instrument 1220. In some examples, this instrument 1210 is a second gripper.

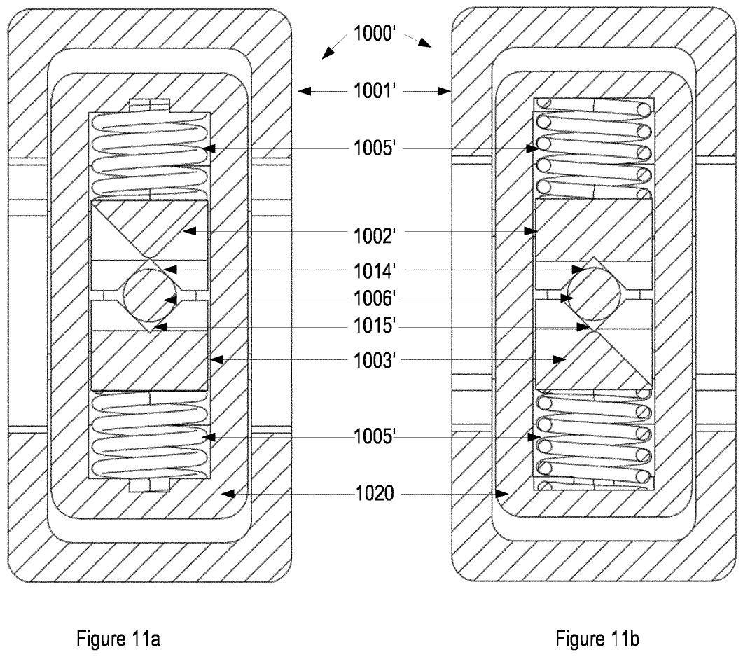

FIG. 11a and b show a cross-sectional view of a schematic illustration of parts of the gripper according to some example implementations as described herein.

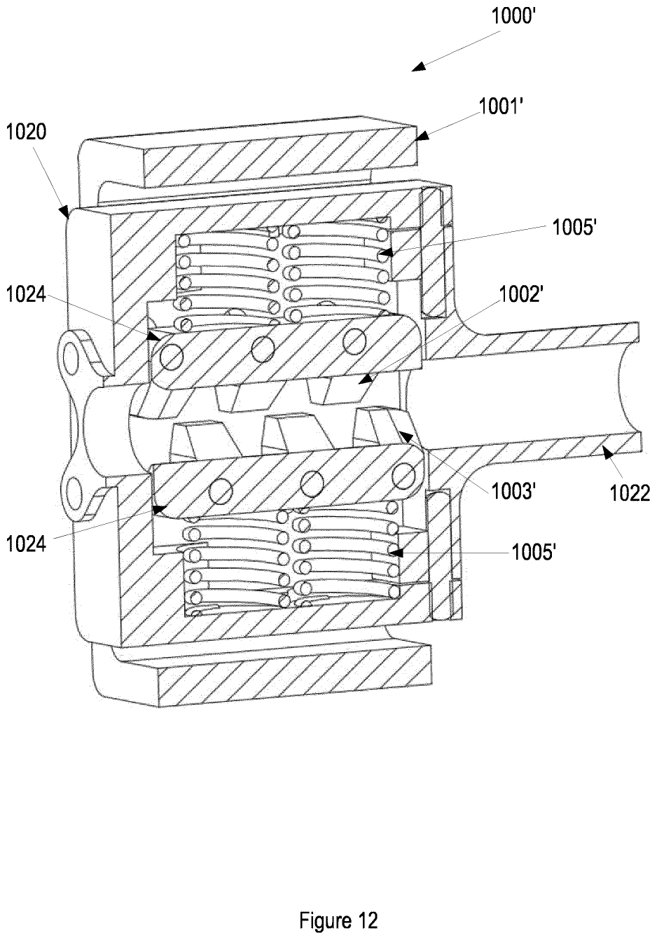

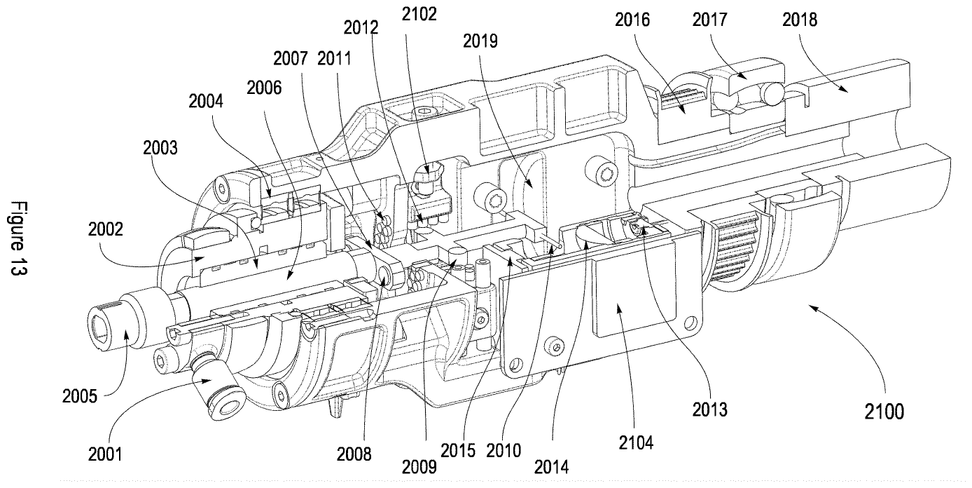

In FIGS. 11a, 11b and 12, features which are substantially similar to the features of the gripper of FIGS. 5 to 10 are indicated by the same reference numeral, but with a “” after it. For example, the spring of FIGS. 11a, 11b and 12 are marked with 1005′. Any characteristics of these features mentioned in relation to FIGS. 11a, 11b and 12 can also apply to the corresponding features of FIGS. 5 to 10, and vice versa.

In the gripper 1000′ of FIGS. 11a and 11b, there is a guide 1001′ configured to guide not only the first and second gipping components 1002′, 1003′, but also an internal guide 1020. This is described in more detail below. In the embodiments of FIGS. 11a, 11b and 12, both the first and second gripping components 1002′, 1003′ are coupled to springs 1005′ and therefore, both the first and second gripping components 1002′, 1003′ are moveable in the same manner as the second gripping component 1003 of FIGS. 5 to 10. As both the first and second gripping components 1002′, 1003′ are moveable, the inner guide 1020 is configured to guide at least a portion of the first gripping component 1002′ and at least a portion of the second gripping component 1003′. The inner guide 1020 may also be configured to guide a least a portion of at least one of the springs 1005′. This may allow for parts of the gripper 1000′ to be guided in a manner which reduces unwanted stress and shear forces during operation of the gripper 1000′, thereby extending the lifetime of the gripper 1000′. In some examples, the inner guide 1020 guides only one, or some, of the first gripping component 1002′, the second gripping component 1003′ and at least one of the plurality of springs 1005′. The first and second gripping components 1002′, 1003′ may be at least partially located within the inner guide 1020, the inner guide 1020 may be located within the guide 1001′, and the inner guide 1020 may be moveable with respect to the guide 1001′.

The inner guide 1020 may be moveable with respect to the guide 1001′ in order to allow for fewer stress and shear forces to be experienced by the elongated member 1006′. In this case, the combination of the inner guide 1020 and the guide 1011′ may act as a form of suspension and cushioning for the elongated member 1006′ gripped by the gripper 1000′. Alternatively, the inner guide 1020 may be fixedly coupled to the guide 1001′ via pins 1004 such as those described above. Additionally or alternatively, the first or second gripping component 1002′, 1003′ may be stationary, or substantially stationary, with respect to the guide 1001′. This may be achieved via said pins 1004.

Additionally, the first and second gripping components 1002′, 1003′ have respective recesses 1014′, 1015′ that allow for the elongated member 1006′ within the gripper 1000′ to be gripped, similar to the recesses 1014, 1015 described above. In some examples, there may be a protrusion 1016 instead of a recess 1014, 1015, as described above.

Similar to the gripper 1000 of FIGS. 5 to 10, the gripper 1000′ of FIGS. 11a, 11b and 12 is configured to grip the elongated member 1006′ via at least a portion of the first gripping component 1002′ and a portion of the second gripping component 1003′ when at least one of the springs 1005′ coupled to the first or second gripping component 1002′, 1003′ is actuated on by the actuating component (not shown in these figures).

The first and second gripping components 1002′, 1003′ coupled to their respective springs 1005′ may have substantially cuboidal or prismatic designs. That is to say, the first gripping component may have a substantially prismatic design whereas the second gripping component may have a substantially cuboidal design, and vice versa. However, any suitable design in any suitable combination may be used for the first and second gripping components 1002′, 1003′. The recesses 1014′, 1015′ may be incorporated into such designs. In some examples, the first and second gripping components 1002′, 1003′ may be of the cuboidal or prismatic design, and be coupled to a secondary component that comprises the recess 1014′, 1015′.

FIG. 12 shows a cut-away view of a schematic illustration of the gripper according to some example implementations as described herein.

In the gripper 1000′ of FIG. 12, most components are substantially the same when compared to the gripper of FIGS. 11a and 11b. the gripper 1000′ of FIG. 12 further comprises a guide tube 1022. This guide tube 1022 may be of a suitable dimension to accept the elongated member 1006′. The elongated member 1006′ can then be guided towards the center of the gripper 1000′ comprising, for example, the first and second gripping components 1002′, 1003′.

In the example of FIG. 12, the first and second gripping components 1002′, 1003′ are each coupled to a corresponding, respective block 1024, which in turn is coupled to a plurality of springs 1005′. In some examples, there may only be one spring 1005′ coupled to at least one of the blocks 1024.

The first and second gripping components 1002′, 1003′ comprise a truncated V-shape, similar to the protrusion 1016 described above, but may have any suitable design. Indeed, at least one of the first and/or second gripping components 1002′, 1003′ may comprise a recess 1014′, 1015′ as described above. However, the first and second gripping components 1002′, 1003′ are offset from each other so that they do not directly contact each other. That is to say, when the first and second gripping components 1002′, 1003′ are moved towards each other, via extension of at least one of the springs 1005′, the first and second gripping components 1002′, 1003′ slot between each other similar to teeth of two gears, or to a zip. This may allow for the elongated member 1006′ within the gripper 1000′ to be gripped in a particularly strong manner. That is to say, in this configuration, when gripped, the elongated member is crimped between the first and second gripping components 1002′, 1003′. Alternatively, the elongated member 1006′ may be gripped in such a way, that when viewed from the side, the elongated member 1006′ has the form of a sine wave or generally a wave-like form. This may allow for the elongated member 1006′ to be gripped in a particularly strong and stable manner. This may also allow for more contact points, thereby further securing the elongated member 1006′ within the gripper 1000′. Alternatively, the first and second gripping components 1002′, 1003′ may contact each other.

The force of the grip may be influenced by the strength of the springs 1005′ and/or the distance that the springs 1005′ are allowed to be extended when acted upon by the actuating component. That is to say, if the springs were extended fully, and, when viewed from the side, the elongated member 1006′ has the form similar to a square wave, the elongated member 1006′ may be very secure. However, this could lead to damage to the elongated member 1006′. Therefore, the extension of the springs 1005′ may be altered based on, for example, the material, fragility and diameter of the elongated member 1006′ to be gripped.

Additionally, in FIG. 12, there are six gripping components 1002′, 1003′ with three gripping components 1002′ being coupled to one of the blocks 1024 and the other three gripping components 1003′ being coupled to the other block 1024. However, it will be realized by the skilled person that there may be any number of gripping components 1002′, 1003′ within the gripper 1000′ and/or that the number of gripping components 1002′, 1003′ coupled to each respective block 1024 may not be equal. This is to say, in a non-limiting example, there may be two gripping components 1002′ coupled to one of the blocks 1024 and four gripping components 1003′ being coupled to the other block 1024.

FIG. 13 shows a cut-away view of a schematic illustration of the sensor 2100 according to example implementations described herein.

The moveable member 2006 extends throughout the sensor. The moveable member 2006 may be made of metal, plastic, carbon fiber or any other suitable material. A wire gripper coupling 2005 is coupled to the moveable member 2006 at one end of the moveable member 2006. The wire gripper coupling 2005 may allow for an elongated endovascular instrument to be coupled to the moveable member 2006. The elongated endovascular instrument may be coupled to the moveable member 2006 by any suitable means. The wire gripper coupling 2005 may additionally or alternatively couple any suitable instrument to the moveable member 2006. In some examples, there is no wire gripper coupling 2005 and the moveable member 2006 is the instrument itself. The wire gripper coupling 2005 may be located at any suitable position on the moveable member 2006. Additionally or alternatively, the elongated endovascular instrument may be an endovascular medical instrument such as a guidewire, stent, balloon, catheter, or any other suitable endovasular instrument.

As the moveable member 2006 enters the sensor 2100, the moveable member 2006 moves through an air bearing 2003. The air bearing 2003 is supplied with air via a compressed air input 2001. The air bearing 2003 is comprised within an air bearing housing 2002. The construction of an air bearing 2003 is known to the skilled person. In some examples, the air bearing 2003 is replaced by a ball bearing, a roller bearing, a magnetic bearing or any other suitable type of bearing. In some examples, there is more than one type of bearing at the entrance to the sensor 2100. In some examples, there is no bearing at the entrance of the sensor 2100. The air bearing may be placed at any suitable position of the sensor 2100 and/or the moveable member 2006.

The moveable member 2006 then comprises a plurality of resilient force members which comprise a resilient force unit 2102 which is described in further detail below.

At the end of the moveable member 2006 opposite to the end which comprises the wire gripper coupling 2005, the moveable member 2006 comprises a needle 2010. The needle 2010 comprises two main sections. One section extends radially from the moveable member 2006 towards the optical unit 2104 which will be described in further detail below. The second section extends radially from the moveable member 2006 towards an oscillation dampening pool 20019. The oscillation dampening pool 20019 may allow for a reduction in unwanted movements of the moveable member 2006. The oscillation dampening pool 2019 may be of any design and may comprise any fluid which allows for the reduction of oscillations. In some examples, the needle 2010 is comprised of two distinct portions coupled via the moveable member 2006. In some examples, the needle 2010 comprises or is one element which travels through the moveable member 2006 and is fixed in place in the moveable member 2006 via any suitable means. The needle 2010 may be of any suitable design which allows for the operation of the sensor. In some examples, the needle 2010 does not extend radially from the moveable member 2006 but in any suitable direction.

The sensor further comprises a pulley 2016 coupled to a stepper motor 2023 which will be described in further detail below.

The sensor comprises a plurality of roller bearings 2004, 2017. The roller bearings 2004, 2017 may allow for the housing 2020 which encloses the sensor 2100 to rotate. The roller bearings 2004, 2017 surrounding the housing 2020 allow the housing 2020 to rotate and measure the torque of the rotation of the housing 2020 in a dynamic manner during continuous rotation of the housing 2020. The rotation of the housing 2020 may also allow for a user of the sensor 2100 to have finer control over the moveable member 2006. The roller bearings 2004, 2017 may be any suitable bearing such as an air bearing, a ball bearing or a magnetic bearing. The roller bearings 2004, 2017 maybe coupled to the housing 2020 at any suitable location and/or via any suitable method. In some examples, there is only one roller bearing 2004, 2017. In some examples, there are no roller bearings 2004, 2017.

The sensor 2100 further comprises, in this example, a slip ring 2018. In some examples, the slip ring 2018 allows for data from the optical unit to be transferred to an external device as will be described in more detail below. In some examples, there is no slip ring 2018.

FIG. 14 shows a perspective view of a schematic illustration of parts of the sensor according to example implementations as described herein.

The sensor 2100 comprises, in this example, a zero positioning unit 2106 and a stepper motor 2023.

The zero positioning unit 2106 comprises a zero position sensor 2021 and a zero position flag 2022. When the sensor is in the zero position i.e. in an unbiased position, the zero position sensor 2021 indicates that the sensor 2100 is in a zero position. The zero position sensor 2021 may comprise a light emitting diode and a photodiode. The zero position flag 2022 may be coupled to the housing 2020 and/or the moveable member 2006. The zero position flag 2022 may be a protrusion from the moveable member 2006 and/or the sensor 2100 and/or the sensor housing 2020 which is configured to pass through the zero position sensor 2021 as the moveable member 2006 and/or the sensor 2100 and/or the sensor housing 2020 is rotated. As the flag 2022 is rotated during rotation of the housing 2020 and/or the moveable member 2006, it will, in certain positions, travel between the light emitting diode and the photodiode. When the flag 2022 is situated between the light emitting diode and the photodiode, the photodiode may send a signal to an indicator to indicate the sensor is in the zero position. The indicator may emit light and/or noise and/or any other suitable emission method to indicate to the user that the sensor is in the zero position. The light emitting diode may alternatively be any suitable light emitting device. The photodiode may alternatively be any suitable electronic device which allows for the detection of light emitted from the light emitting device. The flag 2022 may be of any suitable design which allows for the indication of the zero position. In some examples, there is no zero positioning unit 2106.

The stepper motor 2023 may be coupled to the pulley 2016 shown in FIG. 13. The stepper motor 2023 rotates the sensor housing 2020 through the pulley 2016 to provide feedback to the user of the sensor 2100. For example, if the feedback is set to 0 Nm by a processor of the stepper motor 2023, and a torque is applied to the moveable member 2006, the stepper motor 2023 rotates the sensor housing 2020 to counter act the torque exerted by the user and returns the sensor displacement to 0 Nm. The feedback torque may be set to any suitable torque via the processor. In some examples, the pulley 2016 is a timing belt.

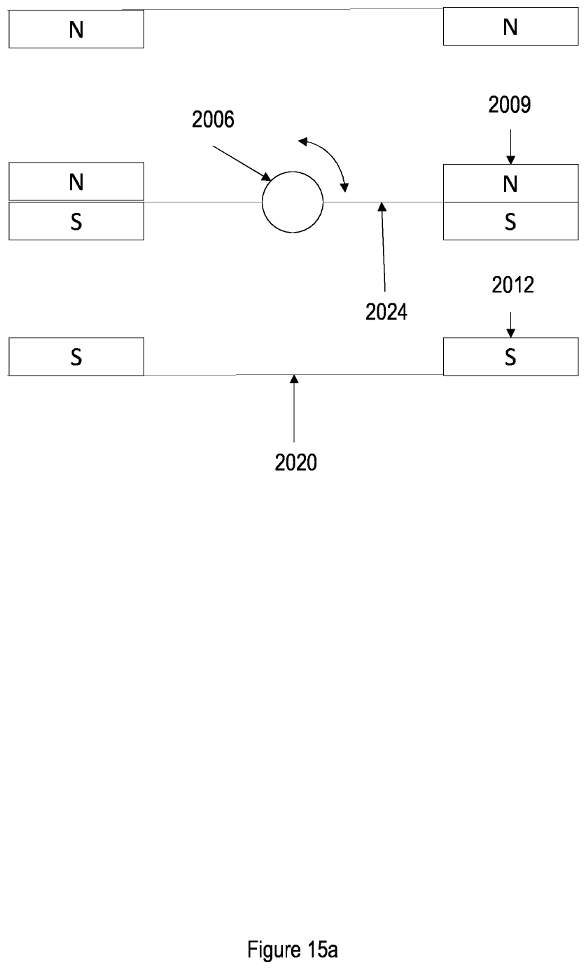

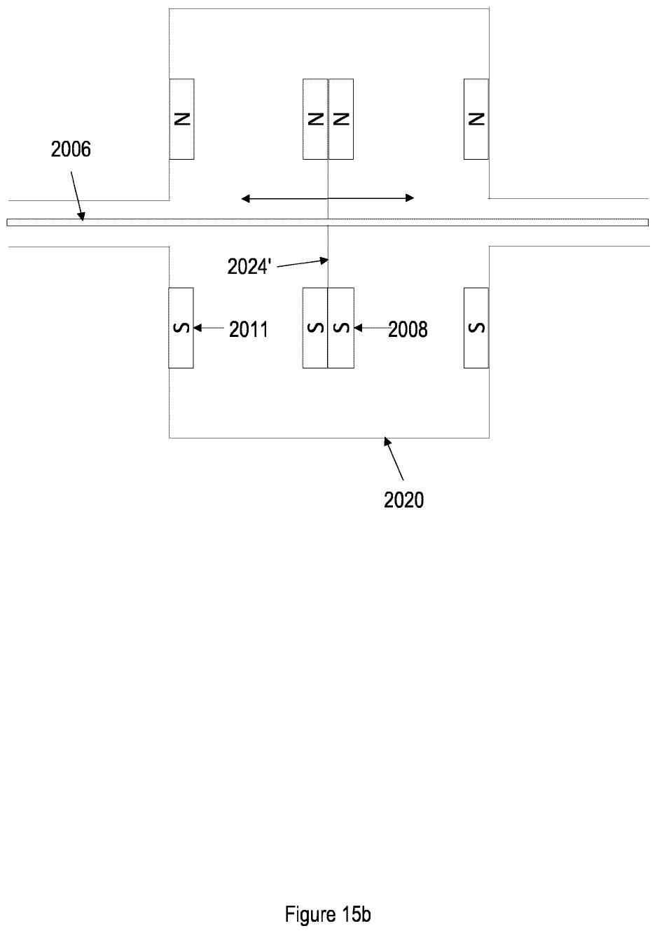

FIGS. 15a and 15b show schematic block diagrams of the resilient force member 2024 according to example implementations as described herein, wherein the resilient force member 2024 is incorporated in the sensor shown in FIGS. 13 and 14.

FIG. 15a shows a schematic block diagram of the rotational resilient force member 2024. In this example, the moveable member 2006 comprises a resilient force member 2024 which is parallel to the axis of rotation of the moveable member 2006. On this resilient force member 2024, there are four magnets 2009. Each of the magnets 2009 on the resilient force member 2024 has a singular polarity. In some examples, there are a different number of magnets. In some examples, one or more of the magnets 2009 on the resilient force member 2024 have a plurality of polarities. A further four magnets 2012 are mounted on the housing 2020 of the sensor 2100. In some examples, these magnets 2012 are merely mounted to a section of the sensor 20100 which is not the moveable member 2006. The magnets 2012 mounted to the housing 2020 are, in this example, stationary and cannot move during operation of the sensor 2100. In this example, each magnet 2012 mounted to the housing 2020 has a corresponding magnet 2009 mounted to the resilient force member 2024 in order to create a magnet pair. Each magnet 2009, 20012 in the magnet pairs has the same polarity so that they repel each other as the moveable member 2006 is rotated in the direction of the arrow. This leads to a nonlinear resilient force being felt by the user of the sensor 2100. This also allows for the moveable member 2006 to be in an unbiased position, i.e. a zero position, when the sensor 2100 is not in use. The strength of the magnets may be altered depending on the use of the sensor 2100. The distance between the magnets 2012 mounted to the housing 2020 and the magnets 2009 on the resilient force member 2024 may be altered via any suitable method such as a screw or a movable platform. In some examples, the strengths of the magnets 2009, 2012 and/or the distance between the magnets 2009, 2012 is not the same for each magnet pair. In some examples, the magnets 2009, 2012 in each magnet pair has opposite polarities.

FIG. 15b shows a schematic block diagram of the linear resilient force member 2024′. The linear resilient force member 2024′ is constructed in substantially the same way as the rotational resilient force member 2024. In this example, the moveable member 2006 comprises an resilient force member 2024′ which is perpendicular to the longitudinal axis of the moveable member 2006 and extends radially from the moveable member 2006. On this resilient force member 2024′, there are four magnets 2008. Each of the magnets 2008 on the resilient force member 2024′ has a singular polarity. In some examples, there are a different number of magnets. In some examples, one or more of the magnets 2008 on the resilient force member 2024′ has a plurality of polarities. A further four magnets 2011 are mounted on the housing 2020 of the sensor 2100. In some examples, these magnets 2011 are merely mounted to a section of the sensor 2100 which is not the moveable member 2006. The magnets 2011 mounted to the housing 2020 are, in this example, stationary and cannot move during operation of the sensor 2100. In this example, each magnet 2011 mounted to the housing 2020 has a corresponding magnet 2008 mounted to the resilient force member 2024′ in order to create a magnet pair. Each magnet 2008, 2011 in the magnet pair has the same polarity so that they repel each other as the moveable member 2006 is moved in the direction of the arrow. This leads to a nonlinear resilient force being felt by the user of the sensor. This also allows for the moveable member 2006 to be in an unbiased position, i.e. a zero position, when the sensor 2100 is not in use. The strength of the magnets may be altered depending on the use of the sensor 2100. The distance between the magnets 2011 mounted to the housing 2020 and the magnets 2008 on the resilient force member 2024′ may be altered via any suitable method such as a screw or a movable platform. In some examples, the strengths of the magnets 2008, 2011 and/or the distance between the magnets 2008, 2011 is not the same for each magnet pair. In some examples, the magnets 2008, 2011 in each magnet pair have opposite polarities.

The resilient force members 2024, 2024′ may be of any design which allows for the operation of the sensor 2100. The resilient force members 2024, 2024′ may comprise plastics, metals, carbon fiber or any other suitable material. The resilient force members 2024, 2024′ described above together comprise the resilient force unit 2102 shown in FIG. 13.

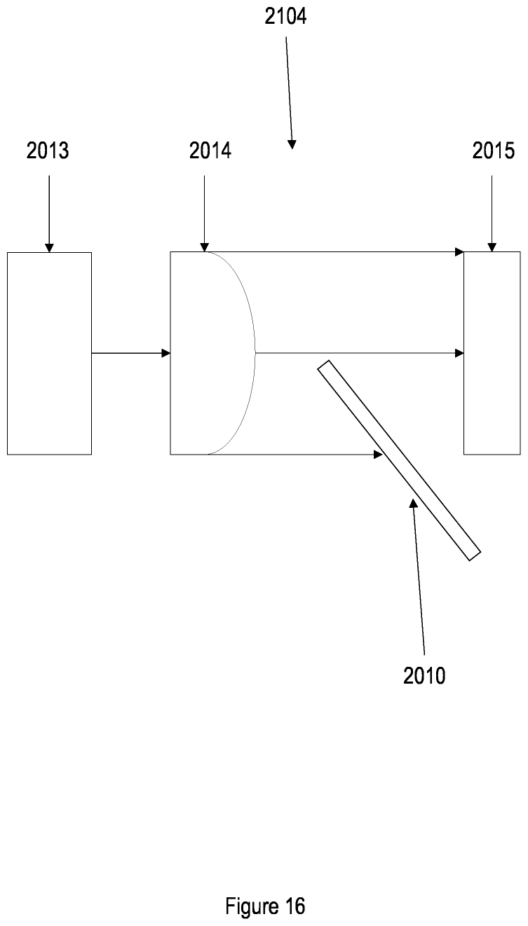

FIG. 16 shows a schematic block diagram of the optical unit according to example implementations as described herein.

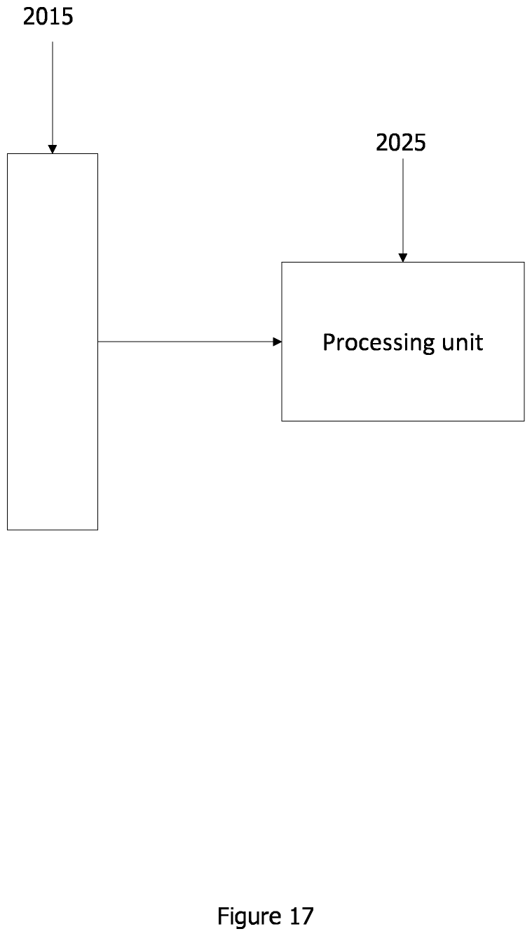

The optical unit 2104 comprises, in this example, three main parts, the light source 2013, the collimating lens 2014, and the light sensor 2015. The light source 2013 is, in this example, a light emitting diode but may additionally or alternatively be any suitable light emitting device. The light source 2013 emits light along an optical path indicated by the arrows in FIG. 16. The optical path then reaches the collimating lens 2014. The collimating lens 2014 is configured to collimate the light of the optical path. After exiting the collimating lens 2014, the optical path reaches the light sensor 2015. The light sensor 2015 is preferably a linear camera with a 1500×1 pixel array, but may be any suitable light sensor 2015. The light sensor 2015 senses the light received by said sensor 2015 and transmits this data to a processing unit (shown in FIG. 17). The processing unit may comprise a processor, a memory, a transceiver or any other suitable component.

Situated between the collimating lens 2014 and the light sensor is the needle 2010 (or generally a light-blocking element). The needle 2010 moves through the optical path depending on the movement of the moveable member 2006 to which the needle 2010 is coupled. Due to the needle 2010 being in the optical path, a portion of the optical path is blocked. The light sensor 2015 is able to sense the amount of light blocked in the optical path by the needle 2010 and/or the areas in which the light is blocked in the optical path by the needle 2010. The light sensor 2015 senses the amount of light not blocked by the needle 2010, and the optical unit 2104 can calculate the amount of the blocked light by knowing the total amount of light emitted by the light source 2013 and the amount of light sensed by the light sensor 2015. In some examples, the motion of the moveable member 2006 can be based (directly) on the amount of light sensed by the light sensor 2015, instead of calculating the amount of light blocked by the needle 2010.

FIG. 17 shows a schematic block diagram of the light sensor and the processing unit according to some example implementations as described herein

The light sensor 2015 transmits the sensed light data to the processing unit 2025. The processor of the processing unit 2025 can then process this data to determine which position the needle 2010 is in. The processor can also determine changes in received data as the needle 2010 is moved from a first position to a second position. Additionally or alternatively, the processor may also be able to calculate the resilient force exerted on the moveable member 2006 by processing the received data. Additionally or alternatively, only the amount of light sensed by the light sensor 2015 (and/or the amount of light blocked by the needle 2010 as calculated based on the amount of light emitted by the light source 2013 and the amount of light sensed by the light sensor 2015) is used further in order to provide feedback data on a movement of the moveable member 2006.

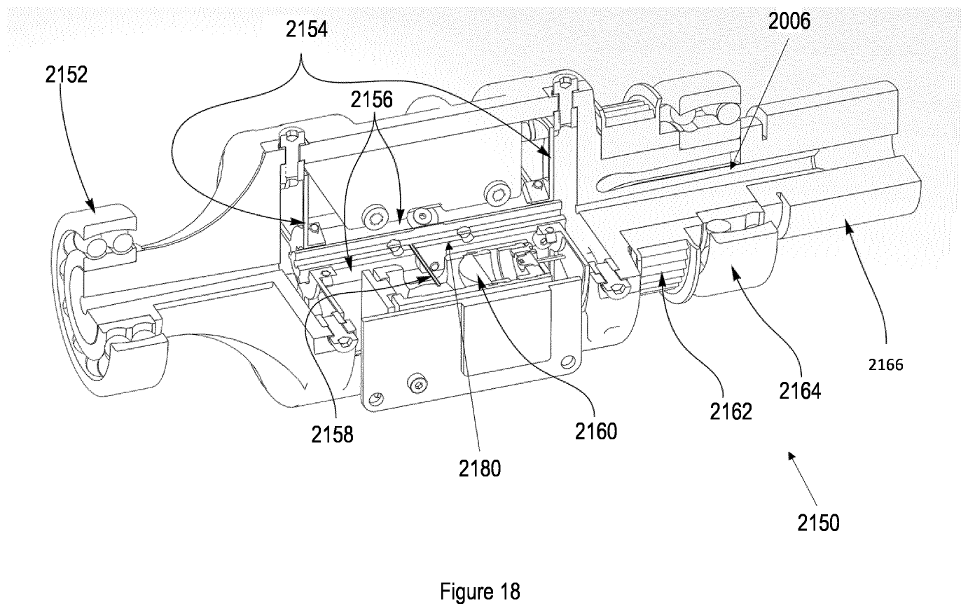

FIG. 18 shows a cut-away view of a schematic illustration of a sensor 2150 according to some example implementations as described herein.

The sensor 2150 shown in FIG. 18 is a variation of the sensor 2100 described above. The sensor 2150 of FIG. 18 comprises a front bearing 2152, an axial displacement elastic component 2154, a rotational displacement elastic component 2156, a displacement needle 2158, an optical sensor 2160, a pulley 2162, a rear bearing 2164, and a slip ring 2166. The resilient force member is, in this example, made up by both of the axial displacement elastic component 2154 and the rotational displacement elastic component 2156.

The axial displacement elastic component 2154 and the rotational displacement elastic component 2156 are both located within an elastic component unit 2180 which will be described in more detail below.

The resilient force member, the displacement needle 2158, the optical sensor 160, the pulley 2162 and the slip ring 2166 may be (substantially) similar or identical to the respective components described above in relation to FIGS. 13 to 17.

In this example, the front bearing 2152 is not an air bearing but is a roller bearing. In some examples, the roller front bearing may be a ball bearing, a magnetic bearing or any other suitable type of bearing which allows for substantially frictionless movement of the moveable member 2006. The rear bearing 2164 may also be a roller bearing but may alternatively be a ball bearing, a magnetic bearing or any other suitable type of bearing which allows for substantially frictionless movement of the moveable member 6.

The axial displacement elastic component 2154 comprises of two parts in this example. The two parts of the axial displacement elastic component 2154 are located at two different sections of the sensor and are positioned along the same axis. That is to say, the two parts are both located substantially along the longitudinal axis of the sensor 2150.

In this example, the two parts of the axial displacement elastic component 2154 comprise four plastic extensions. The four extensions are designed so that they extend from the center of the part in a cross shape i.e. at right angles. The extensions are preferably made of plastic, in particular PETG, but may be made of any other suitable material such as, for example, metal or carbon fiber. In some examples, only a section of one or more of the extensions comprise plastic. There may be any number of extensions in the parts of the axial displacement elastic component 2154. The extensions may be located in any suitable position and any suitable orientation within the two parts of the axial displacement elastic component 2154. In some examples, the extension design within each part is not identical. In some examples, there is only one part in the axial displacement elastic component 2154 and in some examples, there are more than two parts in the axial displacement elastic component 2154.

The extensions have a predetermined flexure which allows for them to elastically deform while providing non-linear haptic feedback to the user of the sensor 2150. That is to say, when the user “pushes” the moveable member 2006, the two parts of the axial displacement elastic component 2154 elastically deform to provide the user with a mechanical resistance force. In some examples, the axial displacement elastic component 2154 further comprises a limiting member which is configured to prevent the axial displacement elastic component 2154 from overdeforming. This may result in a safer sensor 2150 with a longer lifespan.

The rotational displacement elastic component 2156 performs in a similar way to that of the axial displacement elastic component 2154 but in the rotational axis instead of the longitudinal axis.

In this example, the two parts of the rotational displacement elastic component 2156 comprise four plastic extensions extending between the two parts of the axial displacement elastic component 2154. The four extensions are designed so that they extend from along the longitudinal axis of the sensor in a cross shape i.e. at right angles. The extensions are preferably made of plastic, in particular PETG, but may be made of any other suitable material such as, for example, metal or carbon fiber. In some examples, only a section of one or more of the extensions comprise plastic. There may be any number of extensions in the parts of the rotational displacement elastic component 2156. The extensions may be located in any suitable position and any suitable orientation with respect to the two parts of the axial displacement elastic component 2154. In some examples, there are fewer than four extensions in the rotational displacement elastic component 2156 and in some examples, there are more than four extensions in the axial displacement elastic component 2154.

The extensions have a predetermined flexure which allows for them to elastically deform while providing non-linear haptic feedback to the user of the sensor 2150. That is to say, when the user rotates the moveable member 2006, one or more extensions of the rotational displacement elastic component 2156 elastically deform to provide the user with a mechanical resistance force. In some examples, the rotational displacement elastic component 2156 further comprises a limiting member which is configured to prevent the rotational displacement elastic component 2156 from overdeforming. This may result in a safer sensor 2150 with a longer lifespan.

In some examples, one or more extensions of the rotational displacement elastic component 2156 are directly coupled to one or more parts of the axial displacement elastic component 2154. In some examples, one or more of the extensions of the rotational displacement elastic component 2156 are indirectly coupled to one or more parts of the axial displacement elastic component 2154. In some examples, one or more extensions of the rotational displacement elastic component 2156 are coupled to the housing of the sensor 2150.

FIG. 19 shows a perspective view of a schematic illustration of parts of the sensor according to some example implementations as described herein.

The elastic component unit 2180 comprises the axial displacement elastic component 2154 and the rotational displacement elastic component 2156 as described above. In some examples, the elastic component unit 2180 further comprises the displacement needle 2158.

The elastic component unit 2180 further comprises an actuated gripper component 2182, a static gripper component 2184 and a gripper housing 2186.

The actuated gripper component 2182 may comprise any suitable material such as, for example, plastic, metal or carbon fiber. The actuated gripper component 2182 is pushed down via an actuator. The actuator may be a machine or a user pushing down the actuated gripper component 2182. By pushing down on the actuated gripper component 2182, a one or more springs are compressed. In some examples, there is only a single spring and/or any other suitable elastic component such as, for example, rubber.

The static gripper component 2184 is substantially the same as the actuated gripper component 2182 but does, in this example, not have the ability to be actuated.

The moveable member 2006 is placed inside the gripper housing 2186 and through both the actuated and static gripper components 2182, 2184. When the actuated gripper component 2182 is relieved from the actuation motion exerted by a machine or a user, the one or more springs uncompress, pushing the actuated gripper component 2182 up, thereby securing and gripping the moveable member 2006 in place.

The actuated gripper component 2182 may be actuated in any suitable direction depending on the design of the actuated gripper component 2182 i.e. from the side or from below.

In some examples, the elastic component unit 2180 is replaceable. That is, the elastic component unit 2180 is removable from an opening in the sensor 2150 and it can be replaced with another elastic component unit 2180. This may allow for easy alteration of the elasticity of the axial and rotational displacement elastic components 2154, 2156 depending on the parameters of the operation the sensor 2150 is undertaking.

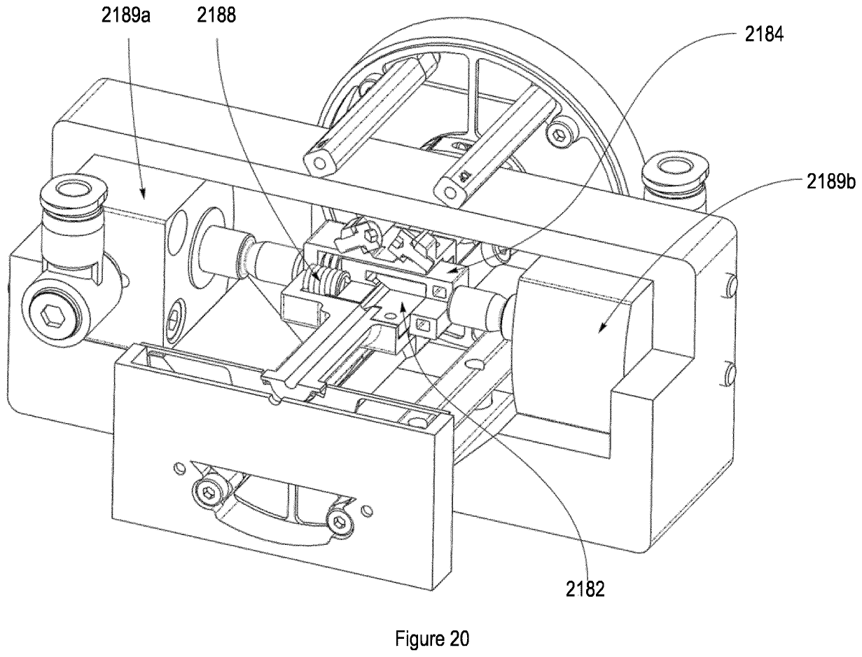

FIG. 20 shows a perspective view of a schematic illustration of a gripper mechanism according to some example implementations as described herein.

The gripper, as described above, comprises an actuated gripper component 2182 and a static gripper component 2184. The actuated gripper component 2182 is biased in a “closed” position, i.e. fixing the moveable member 2006 in place, by a pair of springs 2188. The springs 2188 may be of any suitable strength and comprise any suitable material. In some examples, there is only one spring 2188 or more than three springs 2188. In some examples, there are no springs 2188. In some examples, the springs 2188 are replaced by any suitable elastic component. When the actuated gripper component 2182 needs to be opened, the first cylindrical member 2189a engages with the actuated gripper component 2182 to provide support and the second cylindrical member 2189b contacts the actuated gripper component 2182 in order to force the actuated gripper component 2182 into an “open” position i.e. allowing the moveable member 2006 to move through the actuated gripper component 2182. The cylindrical members 2189a, 2189b may be of any suitable design such as, for example, prismatic or cuboidal. In this embodiment, the cylindrical members 2189a, 2189b are pneumatic cylindrical members 2189a, 2189b. Additionally or alternatively, the cylindrical members 2189a, 2189b may be moveable via electric motors, a generator, electromagnetic means or any other suitable means.

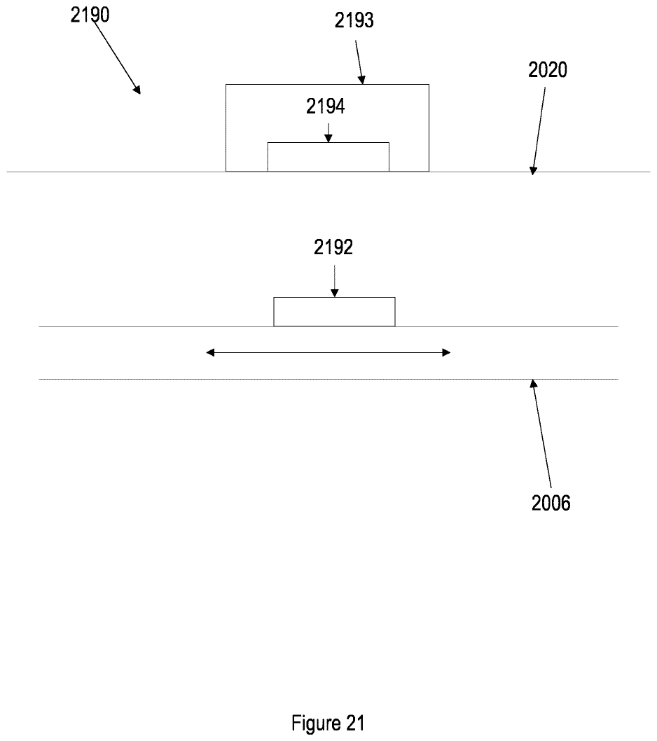

FIG. 21 shows a schematic block diagram of a procedure of detecting the position of the moveable member according to some example implementations as described herein.

FIG. 21 shows an alternate method/procedure 2190 for detecting the position and/or orientation of the moveable member 2006. In this example, a first magnet 2192 is directly coupled to the moveable member. A magnetic field measurement unit 2193 comprises a second magnet 2194 which itself is coupled to the outside of the housing 2020 of the sensor 2100, 2150. This method of detecting the position and/or orientation of the moveable member 2006 can be used additionally or alternately to the method involving the displacement needle 2010, 2158 described above.

The second magnet 2194 may be replaced with a Hall effect sensor, in particular one that conforms to the AS5013 international standard. Within the magnetic field measurement unit 2193, there is a processing unit which comprises a microcontroller and a transmitter. The coupling between the processing unit and the second magnet 2194 is similar to the coupling between the light sensor 2015 and the processing unit 2025 described above in relation to FIG. 17.

In some examples, the first magnet 2192 is not directly coupled to the moveable member 2006 but is coupled to an extension of the moveable member 2006. In some examples, the magnetic field measurement unit 2193 and the second magnet 2194 are coupled to the housing 2020 but located in the inside of the housing 2020. In some examples, the magnetic field measurement unit 2193 and the second magnet 2194 are coupled to a portion of the sensor which is not the housing 2020.

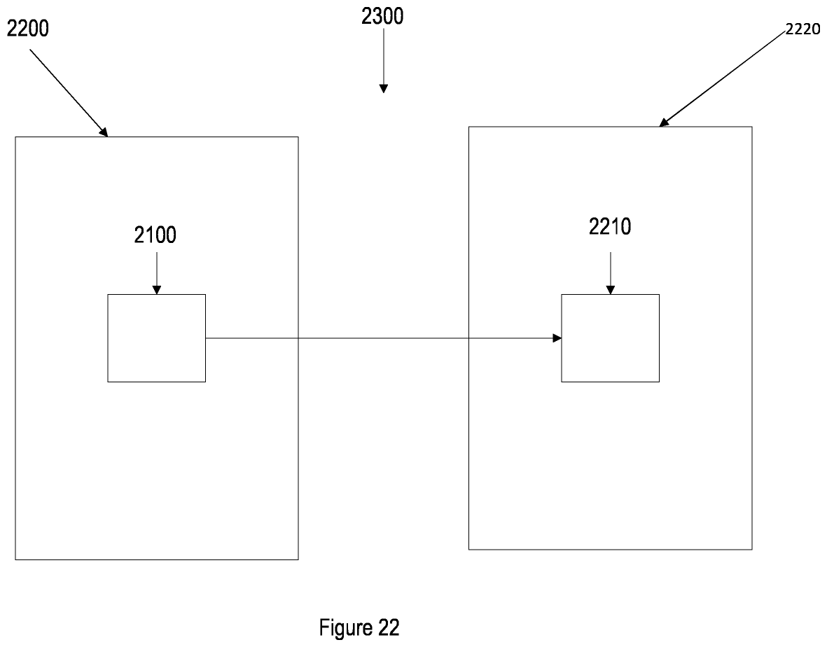

FIG. 22 shows an endovascular robotic system according to some example implementations as described herein.

The processor of the processing unit 2025 described above in relation to FIG. 17 then transmits the positional data of the needle 2010 to the transceiver of the processing unit 2025 which in turn, transmits the data to an external device 2210. The external device 2210 may be a computer, a server, a device with a sensor similar (or identical) to the sensor 2100 described above or any other suitable device. The transceiver may transmit the data via a wired and/or a wireless connection. If the sensor housing 2020 is rotatable and the connection is a wired connection, the data by be transmitted via the slip ring 2018 which allows for data to be safely transmitted between rotatable and fixed objects. If the data is sent to a device with a sensor similar (or identical) to the sensor 2100 described above, the positional data from the sensor 2100 described above may be mimicked in the sensor within the external device 2210. That is to say, the movements of the moveable member 2006 may be translated to a movement of a moveable member of the other sensor, in particular based on data relating to the amount of light blocked by the needle 2010 (calculated based on the amount of light emitted by the light source 2013 from which the amount of light sensed by the light sensor 2015 is subtracted) and/or the amount of light sensed by the light sensor 2015. This may allow for a user to perform an operation from an external location. The data is preferably transmitted via a RS232/RS485 physical connection with proprietary protocol, but any suitable method may be used.

In the example implementation of FIG. 21, the external receiver 2210 is a transceiver located in a second endovascular robotic instrument 2220 and the sensor 2100 is located in a first endovascular robotic instrument 2200. This creates an endovascular robotic system 2300 comprising the sensor 2100, the external device 2210 and the two endovascular robotic instruments 2200, 2220. The movement of the moveable member 2006 within the sensor 2100 is translated to a movement of a moveable member within a second sensor in the second endovascular robotic instrument 2220 by the method described above. This may allow for a user to perform an operation from an external location. In some examples, the movement of the moveable member 2006 may be translated to a movement of a plurality of moveable members within a respective plurality of sensors in a respective plurality of endovascular robotic instruments.

In some examples, the sensor 2100 described above is located on the patient side of an operation. Additionally or alternatively, the sensor 2100 (or an additional said sensor) may be located on the surgeon side of an operation.

In some examples, when the sensor 2100 is on the patient side, the sensor 2100 senses the wire loads inside the patient. The surgeon is then shown the results on a screen and the surgeon controls the guidewire coupled to the moveable member 2006 with a joystick. In some examples, the surgeon has a sensor 2100 which is identical or substantially identical to the sensor on the patient side and controls the patient side sensor via this identical or substantially identical sensor.

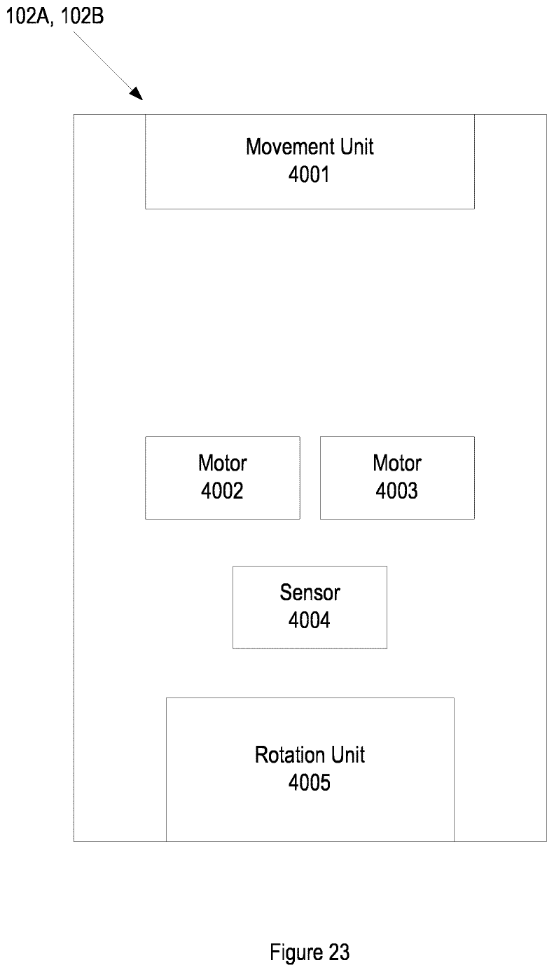

FIG. 23 shows a schematic diagram of a module according to some example implementations as described herein.

FIG. 23 shows that a module 102A, 102B, comprises a movement unit 4001, two motors 4002, 4003, a sensor 4004 and a rotation unit 4005. These items may be the same items as mentioned in relation to the description of FIGS. 1 to 4. In some examples, a module 102A, 102B comprises only some of these features.

FIG. 24 shows a schematic diagram of a system according to some example implementations as described herein.

FIG. 24 shows that each module pair 102, 106, 107 is communicable with its own controller 5102, 5106, 5107, which, in turn, are communicable with a main controller 6000. This may allow for signals to be transmitted and/or received, as described in relation to FIGS. 1 to 4. In some examples, there may be no main controller 6000, and signals are transmitted to the external device and/or received from the external device directly to and/or from a module pair's respective controller 5102, 5106, 5107. Alternatively, at least one module pair 102, 106, 107 may not have its own controller 5102, 5107, 5108, and instead receive and/or transmit signals directly to and/or from the main controller 6000.

The following examples are also encompassed by the present disclosure and may fully or partly be incorporated into embodiments.
1. A medical apparatus for controlling movement of a first elongated medical member, the apparatus comprising:
   a first module and a second module, wherein each of the first and second module comprises a corresponding, respective opening for the first elongated medical member to be passed through the first and second modules, respectively;
   a movement unit configured to move at least one of the first and second modules in a first direction and in a second direction, wherein the first direction is opposite to the second direction;
   wherein at least one of the first and second modules comprises a first gripping unit configured to grip the first elongated medical member passing through the opening of the respective module;
   wherein at least one of the first and second modules comprises a rotation unit configured to rotate the first elongated medical member, passing through the opening of the respective module, about a longitudinal axis of the first elongated medical member;
   wherein the apparatus is configured to operate in a plurality of different modes of operation comprising:
   a) a first mode of operation comprising the first elongated medical member being gripped by the first gripping unit of at least one of the first and second modules and the movement unit moving the at least one of the first and second modules comprising the first gripping unit in the first direction;
   b) a second mode of operation comprising the first elongated medical member being gripped by the first gripping unit of at least one of the first and second modules and the movement unit moving the at least one of the first and second modules comprising the first gripping unit in the second direction;
   c) a third mode of operation comprising the first elongated medical member being gripped by the first gripping unit of at least one of the first and second modules and the rotation unit rotating the first elongated medical member about the longitudinal axis of the first elongated medical member; and
   d) a fourth mode of operation comprising the first or second mode of operation executed simultaneously with the third mode of operation.
2. The apparatus of clause 1, wherein the first gripping unit is configured to be part of a cassette, wherein the cassette comprises the opening suitable for the first elongated medical member.
3. The apparatus of clause 1 or 2, wherein the first gripping unit is rotatable by the rotation unit, and wherein the first elongated medical member is rotatable about its longitudinal axis as the first elongated medical member is gripped by the first gripping unit and the first gripping unit is rotated by the rotation unit.
4. The apparatus of any one of the preceding clauses, the first gripping unit comprising a first gripping component configured to contact at least a first portion of the first elongated medical member on a first side of the first elongated medical member;
   a second gripping component configured to contact at least the first portion of the first elongated medical member on a second side of the first elongated medical member, wherein the first side is different from the second side;
   a guide configured to guide, during a movement of the second gripping component, at least a first portion of the second gripping component; and
   an actuating component coupled to the second gripping component, wherein the second gripping component is moveable between a first position and a second position based on an actuating force provided to the second gripping component via the actuating component, and wherein at least the first portion of the second gripping component is guideable by the guide during movement of the second gripping component between the first position and the second position;
   wherein the first gripping component is configured to stay stationary or substantially stationary with respect to the guide,
   wherein the second gripping component comprises a first surface opposite to a first surface of the first gripping component,
   wherein, when the second gripping component is in the first position, the first elongated medical member is grippable between the first surface of the first gripping component and the first surface of the second gripping component, and wherein, when the second gripping component is in the second position, the first elongated medical member is not grippable between the first surface of the first gripping component and the first surface of the second gripping component.
5. The apparatus of clause 4, wherein the first surface of the first gripping component comprises a first recess configured to accommodate at least the first portion of said first elongated medical member on the first side of said first elongated medical member.
6. The apparatus of clause 4 or 5, wherein the first surface of the second gripping component comprises a second recess configured to accommodate at least the first portion of said first elongated medical member on the second side of said first elongated medical member.
7. The apparatus of clause 4 or 5, wherein the first surface of the second gripping component comprises a protrusion configured to contact at least the first portion of said first elongated medical member on the second side of said first elongated medical member.
8. The apparatus of any one of clauses 4 to 7, wherein the actuating component comprises a resilient member, in particular a spring.
9. The apparatus of any one of clauses 4 to 8, wherein the guide comprises a through hole configured to receive said first elongated medical member between the first surface of the first gripping component and the first surface of the second gripping component.
10. The apparatus of any one of clauses 4 to 9, wherein upon said movement of the second gripping component, based on the actuating force provided to the second gripping component via the actuating component, the first surface of the second gripping component is moveable towards the first surface of the first gripping component.
11. The apparatus of any one of clauses 4 to 10, wherein the first gripping component is at least partially housed by the second gripping component.
12. The apparatus of any one of clauses 4 to 10, wherein the first and second gripping components are at least partially located within an inner guide, wherein the inner guide is located within the guide, and wherein the inner guide is moveable with respect to the guide.
13. The apparatus of clause 12, wherein the first and second gripping components are offset from each other in such a way that when the second gripping component moves between the first position and the second position, the second gripping component does not contact the first gripping component.
14. The apparatus of any one of the preceding clauses, wherein the first elongated medical member is an elongated medical instrument, in particular a catheter, a stent, a catheter balloon, a stent balloon, a thrombectomy device, a coil, a glue system or a guidewire.
15. The apparatus of any one of the preceding clauses, wherein if both the first and second modules comprise movement units, the first and second modules are moveable independent from one another.
16. The apparatus of any one of the preceding clauses, wherein if both the first and second modules comprise first gripping units, the operation of the first gripping units are independent from one another.
17. The apparatus of any one of the preceding clauses, wherein if both the first and second modules comprise rotation units, the operation of the rotation units are independent from one another.
18. The apparatus of any one of the preceding clauses, further comprising a controller configured to control the first gripping unit and/or the movement unit.
19. The apparatus of any one of the preceding clauses, further comprising a sensor, the sensor comprising a moveable member moveable between a first position and a second position,
   a resilient force member coupled to or integral to the moveable member, wherein the resilient force member is configured to provide a resilient force, when the moveable member is in the second position, to bias the moveable member towards the first position, and
   a detection unit configured to detect a change in position of the moveable member from the first position to the second position and/or the second position to the first position.
20. The apparatus of clause 19, wherein the detection unit comprises an optical unit comprising a light source for emitting light and a light sensor for detecting the light emitted by the light source,
   wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor, and
   wherein a first amount of the emitted light which is blockable by the first portion of the moveable member in the optical path between the light source and the light sensor is different between the moveable member being in the first position and the moveable member being in the second position, respectively.
21. The apparatus of clause 20, wherein the optical unit further comprises a lens arranged in the optical path between the light source and the light sensor, and wherein the lens is configured to disseminate the light emitted by the light source.
22. The apparatus of clause 20 or 21, wherein the light source comprises a laser diode.
23. The apparatus of any one of clauses 20 to 22, wherein the sensor is configured to transmit data relating to sensed light stemming from the light source to an external receiver.
24. The apparatus of any one of the preceding clauses, wherein the first elongated medical member is removable from the apparatus while the apparatus is in use, wherein the first elongated medical member is removeable by retracting the first elongated medical member through the openings of the first and second modules.
25. The apparatus of any one of the preceding clauses, in combination with clause 2, wherein the cassette is removeable and replaceable based on the first elongated medical member in the opening.
26. The apparatus of any one of the preceding clauses, wherein the rotation unit comprises a gear and the first elongated medical member is couplable to said gear, and wherein the first elongated medical member is rotatable by the gear through an unlimited rotational angle.
27. The apparatus of any one of the preceding clauses, wherein the first elongated medical member is an over-the-wire type first elongated medical member.
28. A system for controlling a plurality of first elongated medical members, the system comprising:
   a plurality of apparatuses of clause 1;
   wherein each apparatus is for controlling movement of a separate first elongated medical member not controlled by any of the other of the plurality of apparatuses.
29. The system of clause 28, wherein the apparatuses are coaxial to one another.
30. The system of clause 28 or 29, wherein the openings of the first and second modules of each apparatus are coaxial to one another.
31. The system of any one of clauses 28 to 30, wherein the first elongated medical members are telescopically collapsible inside one another.
32. The system of any one of clauses 29 to 31, when dependent on clause 18, wherein the controller of each apparatus is coupled to a main controller.
33. The system of clause 32, wherein the movement units and/or first gripping units of each apparatus are configured to be controlled by the main controller.

34. The system of clause 32 or 33, wherein the movement unit of any apparatus, the first gripping unit of any apparatus and the rotation unit of any apparatus is independently controllable with respect to the movement unit, the first gripping unit and the rotation unit of any other apparatus.
35. The system of any one of clauses 28 to 34, wherein there are three apparatuses.
36. The system of any one of clauses 28 to 35, wherein in the modes of operation of clause 1, when the first elongated medical member is moveable and/or rotatable by at least one of the first and second modules of a first apparatus, at least one of the first and second modules of a second apparatus moves in relation to the at least one of the first and second modules of the first apparatus.
37. The system of any one of clauses 28 to 36, wherein if the first elongated medical member of the first apparatus is gripped, via the first gripping unit, by only one of the first and second modules of the first apparatus, the other one of the first and second modules of the first apparatus is moveable away from the module gripping the first elongated medical member, and at least one of the first and second modules of the second apparatus moves in response to the movement of the module of the first apparatus not gripping the first elongated medical member.
38. A system for controlling movement of a first elongated medical member, the system comprising:
   the apparatus of clause 1; and
   a human control unit comprising a control unit and a second elongated medical member configured to be manipulated by a human;
   wherein the human control unit is located at a first location and the apparatus is located at a second location; and
   wherein the first and second locations are different locations.
39. The system of clause 38, wherein the human control unit comprises a second gripping unit configured to grip the second elongated medical member.
40. The system of clauses 38 or 39, when dependent on clause 18, wherein the control unit is configured to transmit a first control signal to the controller of the apparatus, wherein the first control signal comprises information on the manipulation of the second elongated medical member.
41. The system of clause 40, wherein the controller of the apparatus controls the first elongated medical member in a manner proportional to the manipulation of the second elongated medical member, wherein the controller controls the first elongated medical member based on the received first control signal from the control unit of the human control unit.
42. The system of any one of clauses 38 to 41, when dependent on clause 23, wherein the human control unit further comprises a haptic feedback unit configured to provide haptic feedback to the human manipulating the second elongated medical member based on a sensor unit reading, wherein the haptic feedback is based on the sensor unit reading.
43. The system of any one of clauses 38 to 42, when dependent on clause 40, wherein the human control unit further comprises a pedal actuatable by the human, wherein in a first position, the control unit is configured to transmit the first control signal and in a second position, the control unit is configured not to transmit the first control signal, wherein the first position is different from the second position.
44. The system of any one of clauses 38 to 43, when dependent on clause 40, wherein the human control unit further comprises a touchable device configured to be touchable by the human, wherein upon a first touch of the touchable device, the control unit is configured to transmit the first control signal and upon a second touch, the control unit is configured not to transmit the first control signal.
44. The system of clause 43, wherein upon the first touch, information on only an axial or rotational manipulation of the second elongated medical member is transmitted and upon the second touch, information on only an axial or rotational manipulation of the second elongated medical member is not transmitted.
45. A system for controlling a plurality of first elongated medical members, the system comprising:
the system of clause 28; and
a human control unit comprising a control unit and at least a second elongated medical member and a third elongated medical member, both configured to be manipulated by a human;
wherein the human control unit is located at a first location and the apparatus is located at a second location; and
wherein the first and second locations are different locations.
47. The system of clause 46, wherein the human control unit comprises a second and a third gripping unit configured to grip at least the second and the third elongated medical members, respectively.
48. The system of clauses 46 or 47, when dependent on clause 32, wherein the control unit is configured to transmit a first control signal to the main controller, wherein the first control signal comprises information on the manipulation of at least the second elongated medical member and the third elongated medical member.
49. The system of clause 48, wherein the main controller controls the first elongated medical member in a manner proportional to the manipulation of the second elongated medical member, and a further separate elongated medical member in a manner proportional to the manipulation of the third elongated medical member, wherein the controller controls the first and the further elongated medical members based on the received first control signal from the control unit of the human control unit.
50. The system of any one of clauses 46 to 49, when dependent on clause 23, wherein the human control unit further comprises a haptic feedback unit configured to provide haptic feedback to the human manipulating at least the second and third elongated medical members based on a second control signal transmitted by the detection unit of the sensor, wherein the haptic feedback is proportional to data relating to sensed data of the sensor.
51. The system of any one of clauses 46 to 50, when dependent on clause 48, wherein the human control unit further comprises a pedal actuatable by the human, wherein in a first position, the control unit is configured to transmit the first control signal and in a second position, the control unit is configured not to transmit the first control signal, wherein the first position is different from the second position.
52. The system of any one of clauses 46 to 51, when dependent on clause 48, wherein the human control unit further comprises a touchable device configured to be touchable by the human, wherein upon a first touch of the touchable device, the control unit is configured to transmit the first control signal and upon a second touch, the control unit is configured not to transmit the first control signal.
53. The system of clause 52, wherein upon the first touch, information on only an axial or rotational manipulation of the second and/or third elongated medical member is transmitted and upon the second touch, information on only an axial or rotational manipulation of the second and/or third elongated medical member is not transmitted.
54. The system of clause 44 or 52, wherein the touchable device is a button and/or a lever and/or a touchscreen.
55. A human control unit for manipulating a remote elongated medical member, the human control unit comprising:
   a control unit comprising a transmitter and a receiver;
   a local elongated member configured to be manipulated by a user of the human control unit;
   a gripping unit configured to grip the local elongated member; and
   a device configured to enable and disable the transmitter.
56. The human control unit of clause 55, wherein the device is a button and/or a lever and/or a touchscreen, wherein upon a first touch of the device, the transmitter is enabled and upon a second touch of the device, the transmitter is disabled.
57. The human control unit of clause 55 or 56, further comprising a display configured to display, to the user, a state of the remote elongated member.
58. The human control unit of clause 57, wherein the state comprises a distance the remote elongated member has been manipulated and/or a rotational angle through which the remote elongated member has been manipulated, from a predetermined position.
59. The human control unit of any one of clauses 55 to 58, wherein the transmitter is configured to transmit a first control signal to an external device comprising the remote elongated medical member, wherein the first control signal comprises information on the manipulation of the local elongated member, wherein the human control unit is located in a first location and the external device is located at a second location, wherein the first and second locations are different locations.
60. The human control unit of clause 59, wherein the external device receives the first control signal and the external device manipulates the remote elongated medical member based on the received first control signal.
61. The human control unit of any one of clauses 55 to 60, wherein the control unit is configured to receive, via the receiver, a second control signal, from the external device, wherein the control signal comprises information on the remote elongated medical member.
62. The human control unit of any one of clauses 55 to 60, when dependent on clause 59, further comprising a haptic feedback unit configured to provide haptic feedback to the user of the human control unit based on the received second control signal.
63. The human control unit of any one of clauses 55 to 62, wherein the gripping unit is moveable and/or rotatable based on the manipulation of the local elongated member.
64. The human control unit of any one of clauses 55 to 63, when dependent on clause 61, wherein the gripping unit is moveable and/or rotatable based on the received second control signal.
65. The human control unit of any one of clauses 55 to 64, wherein the human control unit comprises a plurality of local elongated members and the human control unit is for manipulating a plurality of remote elongated medical members.
66. The human control unit of clause 65, wherein the plurality of local elongated members are telescopically collapsible inside one another.
67. The human control unit of any one of clauses 55 to 66, wherein there is an external device for each one of the plurality of local elongated members.
68. The human control unit of any one of clauses 65 to 67, when dependent on clause 57, wherein the display is configured to display, to the user, a state of each of the plurality of local elongated members.
69. The human control unit of any one of clauses 65 to 68, when dependent on clause 59, wherein the first control signal comprises information on the manipulation of each of the plurality of local elongated members.
70. The human control unit of clause 69, wherein the external device receives the first control signal and the external device manipulates the plurality of remote elongated medical members based on the received first control signal.
71. The human control unit of clause 70, wherein each of the plurality of local elongated members corresponds to one of the plurality of remote elongated medical members, wherein the number of local elongated members and the number of remote elongated medical members are equal.
72. The human control unit of any one of clauses 65 to 71, when dependent on clause 62, further comprising a plurality of haptic feedback units configured to provide haptic feedback to the user of the human control unit based on the received second control signal, wherein each of the plurality of haptic feedback units is coupled to a different one of the plurality of local elongated members.
73. The human control unit of any one of clauses 65 to 72, further comprising a plurality of gripping units, wherein each gripping unit is configured to grip a different one of the plurality of local elongated members.
74. The human control unit of clause 73, wherein at least one of the plurality of gripping units is moveable and/or rotatable based on the manipulation of its respective local elongated member.
75. The human control unit of clause 73 or 74, wherein at least one of the plurality gripping units is moveable and/or rotatable based on the received second control signal.

The following examples are additionally encompassed by the present disclosure and may fully or partly be incorporated into embodiments.
1. A gripper for gripping an elongated member of an endovascular system, wherein the gripper comprises:
   a first gripping component configured to contact at least a first portion of a said elongated member on a first side of said elongated member;
   a second gripping component configured to contact at least the first portion of said elongated member on a second side of said elongated member, wherein the first side is different from the second side;
   a guide configured to guide, during a movement of the second gripping component, at least a first portion of the second gripping component; and
   an actuating component coupled to the second gripping component, wherein the second gripping component is moveable between a first position and a second position based on an actuating force provided to the second gripping component via the actuating component, and wherein at least the first portion of the second gripping component is guideable by the guide during movement of the second gripping component between the first position and the second position;
   wherein the first gripping component is configured to stay stationary or substantially stationary with respect to the guide,
   wherein the second gripping component comprises a first surface opposite to a first surface of the first gripping component,
   wherein, when the second gripping component is in the first position, said elongated member is grippable between the first surface of the first gripping component and the first surface of the second gripping component, and
   wherein, when the second gripping component is in the second position, said elongated member is not grippable between the first surface of the first gripping component and the first surface of the second gripping component.
2. The gripper of clause 1, wherein the first surface of the first gripping component comprises a first recess configured to accommodate at least the first portion of said elongated member on the first side of said elongated member.
3. The gripper of clause 2, wherein the first recess is v-shaped.
4. The gripper of any one of the preceding clauses, wherein the first surface of the second gripping component comprises a second recess configured to accommodate at least the first portion of said elongated member on the second side of said elongated member.
5. The gripper of clause 4, wherein the second recess is v-shaped.
6. The gripper of clause 4 or 5, when dependent on clause 2 or 3, wherein at least the first portion of said elongated member is contactable, via the first surface of the first gripping component on the first side of said elongated member and the first surface of the second gripping component on the second side of said elongated member, at four contact lines via the first and second recesses.
7. The gripper of clause 6, wherein the four contact lines are each separated by 90°, and wherein the four contact lines are parallel to one another.
8. The gripper of clause 4 or 5, when dependent on clause 2 or 3, wherein at least the first portion of said elongated member is contactable, via the first surface of the first gripping component on the first side of said elongated member and the first surface of the second gripping component on the second side of said elongated member, at four contact points via the first and second recesses.
9. The gripper of clause 8, wherein the four contact points are each separated by 90°, and wherein the four contact points span a quadrilateral around a central longitudinal axis of said elongated member.
10. The gripper of any one of clauses 1 to 3, wherein the first surface of the second gripping component comprises a protrusion configured to contact at least the first portion of said elongated member on the second side of said elongated member.
11. The gripper of clause 10, wherein the protrusion comprises a truncated v-shape.
12. The gripper of clause 10 or 11, when dependent on clause 2 or 3, wherein at least the first portion of said elongated member is contactable, via the first surface of the first gripping component on the first side of said elongated member and the first surface of the second gripping component on the second side of said elongated member, at two contact lines via the first recess and one contact line via the protrusion.
13. The gripper of clause 12, wherein the three contact lines are each separated by 120°, and wherein the three contact lines are parallel to one another.
14. The gripper of clause 10 or 11, when dependent on clause 2 or 3, wherein at least the first portion of said elongated member is contactable, via the first surface of the first gripping component on the first side of said elongated member and the first surface of the second gripping component on the second side of said elongated member, at two contact points via the first recess and one contact point via the protrusion.
15. The gripper of clause 14, wherein the three contact points are each separated by 120°, and wherein the three contact points span a triangle around a central longitudinal axis of said elongated member.
16. The gripper of any one of the preceding clauses, wherein the actuating component comprises a resilient member, in particular a spring.
17. The gripper of any one of the preceding clauses, wherein the guide comprises a through hole configured to receive said elongated member between the first surface of the first gripping component and the first surface of the second gripping component.
18. The gripper of any one of the preceding clauses, wherein at least the first portion of said elongated member is grippable between the first surface of the first gripping component and the first surface of the second gripping component.
19. The gripper of any one of the preceding clauses, wherein the second gripping component and/or the guide comprises a photopolymer and/or comprises a 3D-printed portion.
20. The gripper of any one of the preceding clauses, wherein the first gripping component comprises a metallic portion, wherein at least a portion of the metallic portion is configured to contact at least the first portion of said elongated member.
21. The gripper of any one of the preceding clauses, wherein upon said movement of the second gripping component, based on the actuating force provided to the second gripping component via the actuating component, the first surface of the second gripping component is moveable towards the first surface of the first gripping component.
22. The gripper of any one of the preceding clauses, further comprising an assembly pin configured to couple
   (i) the first gripping component and the guide to each other, and/or
   (ii) the first gripping component and a component external to the gripper to each other, and/or
   (iii) the guide and the component external to the gripper to each other.
23. The gripper of any one of the preceding clauses, wherein the guide comprises a first portion and a second portion, wherein the first portion is configured to guide at least the first portion of the second gripping component and to guide at least the first portion of said elongated member, wherein the second portion of the guide comprises a section configured to guide at least a second portion of said elongated member, and wherein the first and second portions of said elongated member are distinct portions.
24. The gripper of any one of the preceding clauses, wherein the gripper is situated in a disposable cassette.
25. The gripper of any one of the preceding clauses, wherein the first gripping component and/or the second gripping component and/or the guide are at least partially coated in a polymer.
26. The gripper of any one of the preceding clauses, wherein the actuating force provided to the second gripping component via the actuating component is dependent on at least one parameter of said elongated member.
27. The gripper of any one of the preceding clauses, wherein said elongated member is a guidewire and/or a catheter.
28. The gripper of any one of the preceding clauses, when dependent on clause 22, further comprising a sensor configured to indicate to the external component an actuation of the actuating component.
29. The gripper of any one of the preceding clauses, wherein the first gripping component is at least partially housed by the second gripping component.
30. The gripper of any one of the preceding clauses, wherein the guide is further configured to guide at least a portion of the actuating component and/or at least a first portion of the first gripping component.
31. The gripper of any one of the preceding clauses, wherein the second gripping component is slideable past the first gripping component and the guide during the movement of the second gripping component between the first position and the second position and/or between the second position and the first position.
32. The gripper of any one of the preceding clauses, wherein the movement of the second gripping component towards the second position is limitable upon a contact between the first gripping component and the second gripping component.
33. The gripper of any one of the preceding clauses, wherein the movement of the second gripping component towards the first position is limitable upon a contact between the second gripping component and said elongated member.
34. The gripper of any one of the preceding clauses, when dependent on any one of clauses 2 to 5, wherein a movement of said elongated member is limitable by the first recess and/or the second recess when the second gripping component is in the second position.
35. The gripper of any preceding clauses, when dependent on clause 20, wherein the metallic portion comprises 7075-t6 aluminum.
36. An endovascular system comprising:
   a first endovascular instrument; and
   a second endovascular instrument;
   wherein the first and/or second endovascular instrument comprises a gripper according to any one of clauses 1 to 35.
37. The endovascular system of clause 36, further comprising the elongated member, wherein the elongated member is a guidewire and/or a catheter.
38. The endovascular system of clause 36 or 37, wherein the first endovascular instrument is a first robotic endovascular instrument and/or the second endovascular instrument is a second robotic endovascular instrument.
39. A sensor for an endovascular robotic system, wherein the sensor comprises:
   a moveable member moveable between a first position and a second position,
   a resilient force member coupled to or integral to the moveable member, wherein the resilient force member is configured to provide a resilient force, when the moveable member is in the second position, to bias the moveable member towards the first position, and
   a detection unit configured to detect a change in position of the moveable member from the first position to the second position and/or the second position to the first position.
40. The sensor of clause 39, wherein the detection unit comprises an optical unit comprising a light source for emitting light and a light sensor for detecting the light emitted by the light source,
   wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor, and
   wherein a first amount of the emitted light which is blockable by the first portion of the moveable member in the optical path between the light source and the light sensor is different between the moveable member being in the first position and the moveable member being in the second position, respectively.
41. The sensor of clause 40, wherein the sensor is configured to determine that the moveable member is in the first position and/or the second position based on a second amount of the emitted light, sensed by the light sensor, not blockable by the first portion of the moveable member in the optical path between the light source and the light sensor when the moveable member is in the first position and/or second position.
42. The sensor of clause 41, wherein the sensor is configured to determine a magnitude of the resilient force based on the second amount of the emitted light sensed by the light sensor.
43. The sensor of any one of clauses 40 to 42, wherein the sensor is configured to determine that the moveable member is in
   a first transition between the first position and the second position and/or a
   second transition between the second position and the first position based on a change of a said amount of the emitted light not blockable by the first portion of the moveable member in the optical path between the light source and the light sensor.
44. The sensor of any one of clauses 39 to 43, wherein the resilient force is a nonlinear resilient force, and wherein the nonlinear resilient force is configured to change nonlinearly as the moveable member moves between the first position and the second position and/or between the second position and the first position.
45. The sensor of any one of clauses 39 to 44, wherein the resilient force is a continuous resilient force, and wherein the continuous resilient force is configured to change continuously as the moveable member moves between the first position and the second position and/or between the second position and the first position.
46. The sensor of any one of clauses 40 to 43, or clause 44 or 45 when dependent from clause 40, wherein the optical unit further comprises a lens arranged in the optical path between the light source and the light sensor, and wherein the lens is configured to disseminate the light emitted by the light source.
47. The sensor of any one of clauses 39 to 46, wherein the moveable member is moveable in two degrees of freedom.
48. The sensor of clause 47, wherein the first degree of freedom is along an axial direction of the moveable member, and wherein the second degree of freedom relates to a rotational axis about the axial direction.
49. The sensor of any one of clauses 39 to 48, wherein the resilient force member comprises a plurality of magnets, wherein a first magnet of the plurality of magnets is coupled to the moveable member, and wherein a second magnet of the plurality of magnets is coupled to a part of the sensor different from the moveable member, and wherein the resilient force comprises a magnetic force between the first magnet and the second magnet.
50. The sensor of clause 49, wherein the plurality of magnets are arranged such that, in a pair of magnets, the magnets repel or attract each other, and wherein the pair of magnets biases the moveable member towards the first position.
51. The sensor of clause 49 or 50, wherein a distance between at least two of the plurality of magnets is configured to change based on a movement of the moveable member, and wherein the change in distance between the at least two of the plurality of magnets is configured to change the resilient force.
52. The sensor of any one of clauses 49 to 51, when dependent on clause 9 or 10, wherein a first plurality of the plurality of magnets is configured to provide a change in a first directional resilient force in relation to a first degree of freedom as the moveable member is moved between the first position and the second position and/or between the second position and the first position, and wherein a second plurality of the plurality of magnets is configured to provide a change in a second directional resilient force in relation to a second degree of freedom as the moveable member is moved between the first position and the second position and/or between the second position and the first position.
53. The sensor of any one of clauses 40 to 43 or 46, or clause 44 or 45 or 47 to 52 when dependent from clause 40, wherein the light source comprises a laser diode.
54. The sensor of any one of clauses 39 to 53, wherein a first portion of the resilient force member is coupled to the moveable member, and wherein the first portion of the resilient force member is configured to contact a first elastic component coupled to a part of the sensor different from the moveable member, and wherein the resilient force comprises a mechanical resistance force between the first portion of the resilient force member and the first elastic component.
55. The sensor of clause 54, wherein the first portion of the resilient force member and the first elastic component are arranged such that the first portion of the resilient force member and the first elastic component bias the moveable member towards the first position.
56. The sensor of clause 54 or 55, when dependent on clause 47 or 48, wherein the first portion of the resilient force member and the first elastic component are configured to provide a change in a first directional resilient force in relation to a first degree of freedom as the moveable member is moved between the first position and the second position and/or between the second position and the first position, and wherein a second portion of the resilient force member and a second elastic component are configured to provide a change in a second directional resilient force in relation to a second degree of freedom as the moveable member is moved between the first position and the second position and/or between the second position and the first position.
57. The sensor of any one of clauses 39 to 56, wherein a second portion of the moveable member is arranged within an oscillation dampening pool, wherein the oscillation dampening pool is configured to provide oscillation dampening to the moveable member.
58. The sensor of any one of clauses 39 to 57, further comprising an air bearing, wherein a third portion of the moveable member is arranged at least partially in the air bearing, and wherein the air bearing is configured to allow the moveable member to move in a substantially frictionless manner.
59. The sensor of any one of clauses 39 to 58, further comprising a zero positioning unit, wherein the zero positioning unit comprises a positioning sensor, a positioning flag and an indicator configured to indicate that the sensor is in a zero position, wherein the moveable member does not encounter a net force in the zero position.
60. The sensor of any one of clauses 39 to 59, further comprising a sensor housing and a sensor bearing coupled to the sensor housing, and wherein the sensor bearing is configured to allow the sensor housing to rotate about a longitudinal axis of the sensor housing.
61. The sensor of any one of clauses 39 to 60, wherein the moveable member is coupled to or comprises a first endovascular robotic instrument.
62. The sensor of any one of clauses 39 to 61 in combination with clause 40, wherein the sensor is configured to transmit data relating to sensed light stemming from the light source to an external receiver.
63. The sensor of clause 62, wherein the external receiver is comprised in a second endovascular robotic instrument controllable based on the received data.
64. The sensor of clause 62 or 63, further comprising a slip ring, wherein the slip ring is configured to allow for substantially continuous transmission of data corresponding to the sensed light to the external receiver.
65. The sensor of 63 or 64, when dependent on clause 61, wherein the second endovascular robotic instrument is identical or substantially identical to the first endovascular robotic instrument, and/or wherein a function of the second endovascular robotic instrument is identical to a function of the first endovascular robotic instrument.
66. The sensor of any one of clauses 39 to 65, wherein the detection unit comprises a magnetic field measurement unit configured to detect a change in a magnetic field,
   wherein in the first and/or second position of the moveable member, a characteristic of the magnetic field is detectable by the magnetic field measurement unit, and
   wherein the characteristic of the magnetic field detectable by the magnetic field measurement unit is a first value when the moveable member is in the first position and a second value when the moveable member is in the second position, wherein the first value is different from the second value.
67. The sensor of clause 66, wherein the sensor is configured to determine that the moveable member is in the first position and/or the second position based on the characteristic detectable by the magnetic field measurement unit when the moveable member is in the first position and/or second position.
68. The sensor of clause 67, wherein the sensor is configured to determine a magnitude of the resilient force based on the characteristic detectable by the magnetic field measurement unit when the moveable member is in the second position.
69. The sensor of any one of clauses 66 to 68, wherein the sensor is configured to determine that the moveable member is in
   a first transition between the first position and the second position and/or a
   second transition between the second position and the first position based on a change of the characteristic detectable by the magnetic field measurement unit.
70. The sensor of any one of clauses 66 to 69, wherein the magnetic field measurement unit is configured to transmit data about the detected characteristic to a microcontroller coupled to the magnetic field measurement unit.
71. the Sensor of Clause 70, Wherein the Magnetic Field Measurement Unit comprises a first magnet.
72. The sensor of any one of clauses 66 to 71, wherein the magnetic field measurement unit comprises a Hall effect sensor.
73. The sensor of clause 71 or 72, further comprising a second magnet coupled to the moveable member, and wherein the characteristic detectable by the magnetic field measurement unit is based on a magnetic field interaction between the first magnet and the second magnet.
74. A sensor for an endovascular robotic system, wherein the sensor comprises:
   a moveable member moveable between a first position and a second position, and
   an optical unit comprising a light source for emitting light and a light sensor for detecting the light emitted by the light source,
   wherein a portion of the moveable member is arranged, in the first and/or second position of the moveable member, in an optical path of the emitted light between the light source and the light sensor for at least partially blocking, by the portion of the moveable member, the emitted light travelling on the optical path between the light source and the light sensor, and
   wherein a first amount of the emitted light which is blockable by the portion of the moveable member in the optical path between the light source and the light sensor is different between the moveable member being in the first position and the moveable member being in the second position, respectively, and
   wherein the light sensor is configured to determine that the moveable member is in the first position and/or the second position based on a second amount of the emitted light, sensed by the light sensor, not blockable by the portion of the moveable member in the optical path when the moveable member is in the first position and/or second position.
75. A sensor for an endovascular robotic system, wherein the sensor comprises:
   a moveable member moveable between a first position and a second position, and
   a magnetic field measurement unit comprising a first magnet, wherein the magnetic field measurement unit is configured to detect a change in a magnetic field,
   wherein in the first and/or second position of the moveable member, a characteristic of the magnetic field is detectable by the magnetic field measurement unit,
   wherein the characteristic of the magnetic field detectable by the magnetic field measurement unit is a first value when the moveable member is in the first position and a second value when the moveable member is in the second position, wherein the first value is different from the second value, and
   wherein the sensor is configured to determine that the moveable member is in the first position and/or the second position based on the characteristic detectable by the magnetic field measurement unit when the moveable member is in the first position and/or second position.
76. An endovascular robotic system, comprising:
   a first endovascular robotic instrument located at a first location, and
   a second endovascular robotic instrument located at a second location different from the first location,
   wherein the first endovascular robotic instrument is communicatively coupled with the second endovascular robotic instrument, and
   wherein the first endovascular robotic instrument and/or the second endovascular robotic instrument comprises the sensor of any one of clauses 39 to 75.
77. The endovascular robotic system of clause 76, wherein a first functioning of the first endovascular robotic instrument is identical to a second functioning of the second endovascular robotic instrument,
   wherein the first endovascular robotic instrument comprises a first haptic feedback unit configured to generate first haptic feedback data dependent on a first movement, for implementing the first functioning, of the first endovascular robotic instrument,
   wherein the first endovascular robotic instrument is configured to send the first haptic feedback data to the second endovascular robotic instrument, and
   wherein the second endovascular robotic instrument is configured to mimic, for implementing the second functioning, the first movement of the first endovascular robotic instrument based on the first haptic feedback data received from the first endovascular robotic instrument.
78. The endovascular robotic system of clause 77, wherein the first endovascular robotic instrument comprises the sensor of any one of clauses 39 to 75, and wherein the first endovascular robotic instrument is configured to generate the first haptic feedback data based on the amount of the emitted light detected by the light sensor.

It is to be understood that terms such as “controls”, “transmits”, “receives”, “manipulates” and the like within the present writ should also be understood be mean that the feature is configured to be controlled and/or manipulated and that a signal is configured to be transmitted and/or received and so on.

No doubt many other effective alternatives will occur to the skilled person. It will be understood that the invention is not limited to the described embodiments and encompasses modifications apparent to those skilled in the art and lying within the scope of the claims appended hereto.

1. A human control unit for manipulating a remote elongated medical member, the human control unit comprising:
a control unit comprising a transmitter;
a local elongated member configured to be manipulated by a user of the human control unit;
a gripping unit configured to grip the local elongated member; and
a device configured to enable and disable the transmitter.

2. The human control unit of claim 1, wherein the device is a button and/or a lever and/or a touchscreen, wherein upon a first touch of the device, the transmitter is configured to be enabled and upon a second touch of the device, the transmitter is configured to be disabled, wherein the first and second touches occur at two different temporal instances.

3. The human control unit of claim 1 further comprising a display configured to display, to the user, a state of the remote elongated member, in particular wherein the state comprises a distance the remote elongated member has been manipulated and/or a rotational angle through which the remote elongated member has been manipulated, from a predetermined position.

4. (canceled)

5. The human control unit of claim 1, wherein the transmitter is configured to transmit a first control signal to an external device comprising or being coupled to the remote elongated medical member, wherein the first control signal comprises information on the manipulation of the local elongated member, wherein the human control unit is located in a first location and the external device is located at a second location, wherein the first and second locations are different locations, in particular wherein the external device is configured to receive the first control signal and the external device is configured to manipulate the remote elongated medical member based on the received first control signal.

6. (canceled)

7. The human control unit of claim 1, wherein the control unit is configured to receive, via a receiver, a second control signal, from the external device, wherein the control signal comprises information on the remote elongated medical member, in particular wherein the gripping unit is moveable and/or rotatable based on the received second control signal, more particularly wherein the human control unit further comprises a haptic feedback unit configured to provide haptic feedback to the user of the human control unit based on the received second control signal.

8. (canceled)

9. (canceled)

10. (canceled)

11. The human control unit of claim 1, wherein the human control unit comprises a plurality of local elongated members and the human control unit is configured to manipulate a plurality of remote elongated medical members, in particular wherein the plurality of local elongated members are telescopically collapsible inside one another.

12. (canceled)

13. (canceled)

14. (canceled)

15. The human control unit of claim 11, wherein the transmitter is configured to transmit a first control signal to an external device comprising or being coupled to the remote elongated medical member, wherein the first control signal comprises information on the manipulation of the local elongated member, wherein the human control unit is located in a first location and the external device is located at a second location, wherein the first and second locations are different locations wherein the first control signal comprises information on the manipulation of each of the plurality of local elongated members,
in particular wherein the external device is configured to receive the first control signal and the external device is configured to manipulate the plurality of remote elongated medical members based on the received first control signal,
more particularly wherein each of the plurality of local elongated members corresponds to one of the plurality of remote elongated medical members, wherein the number of local elongated members and the number of remote elongated medical members are equal.

16. (canceled)

17. (canceled)

18. The human control unit of claim 11, further comprising a plurality of haptic feedback units configured to provide haptic feedback to the user of the human control unit based on the received second control signal, wherein each of the plurality of haptic feedback units is couplable to a different one of the plurality of local elongated members.

19. The human control unit of claim 11, further comprising a plurality of gripping units, wherein each gripping unit is configured to grip a different one of the plurality of local elongated members, in particular wherein at least one of the plurality of gripping units is moveable and/or rotatable based on the manipulation of its respective local elongated member, and/or wherein at least one of the plurality gripping units is moveable and/or rotatable based on the received second control signal.

20. (canceled)

21. (canceled)

22. A system for controlling movement of a first elongated medical member, the system comprising:
an external apparatus; and
a human control unit comprising a control unit and a second elongated medical member configured to be manipulated by a human, wherein the human control unit comprises a first gripping unit configured to grip the second elongated medical member;
wherein the human control unit is located at a first location and the apparatus is located at a second location; and
wherein the first and second locations are different locations.

23. The system of claim 22, the external apparatus comprising a controller configured to control a second gripping unit and/or a movement unit of the external apparatus, wherein the control unit is configured to transmit a first control signal to the controller of the external apparatus, wherein the first control signal comprises information on the manipulation of the second elongated medical member, in particular wherein the controller of the external apparatus is configured to manipulate the first elongated medical member in a manner proportional to the manipulation of the second elongated medical member, wherein the controller is configured to control the first elongated medical member based on the received first control signal from the control unit of the human control unit.

24. (canceled)

25. The system of claim 22, the external apparatus comprising a sensor configured to transmit sensed data to the control unit, wherein the human control unit further comprises a haptic feedback unit configured to provide haptic feedback to the human manipulating the second elongated medical member based on a second control signal transmitted by a detection unit of the sensor, wherein the haptic feedback is proportional to sensed data.

26. The system of claim 23, wherein the human control unit further comprises a pedal actuatable by the human, wherein in a first position, the control unit is configured to transmit the first control signal and in a second position, the control unit is prohibited from transmitting the first control signal, wherein the first position is different from the second position.

27. : The system of claim 23, wherein the human control unit further comprises a touch device, wherein upon a first touch of the touch device, the control unit is configured to transmit the first control signal and upon a second touch, the control unit is prohibited from transmitting the first control signal, wherein the first and second touches occur at two different temporal instances.

28. The system of claim 27, wherein upon the first touch, information on only an axial or rotational manipulation of the second elongated medical member is transmittable and upon the second touch, information on only an axial or rotational manipulation of the second elongated medical member is not transmittable.

29. A system for controlling a plurality of first elongated medical members, the system comprising:
an external system; and
a human control unit comprising a control unit and at least a second elongated medical member and a third elongated medical member, both configured to be manipulated by a human;
wherein the human control unit is located at a first location and the external system is located at a second location; and
wherein the first and second locations are different locations.

30. (canceled)

31. The system of claim 29, wherein the external system comprises a main controller, wherein the control unit is configured to transmit a first control signal to the main controller, wherein the first control signal comprises information on the manipulation of at least the second elongated medical member and the third elongated medical member, in particular wherein the main controller is configured to control the first elongated medical member in a manner proportional to the manipulation of the second elongated medical member, and a further separate elongated medical member in a manner proportional to the manipulation of the third elongated medical member, wherein the main controller is configured to control the first and the further elongated medical members based on the received first control signal from the control unit of the human control unit.

32. (canceled)

33. The system of claim 29, wherein at least one of the plurality of gripping units is moveable and/or rotatable based on the received second control signal, wherein the external system comprises a sensor configured to transmit sensed data to the control unit, wherein the human control unit further comprises a haptic feedback unit configured to provide haptic feedback to the human manipulating at least the second and third elongated medical members based on a second control signal transmitted by a detection unit of the sensor, wherein the haptic feedback is proportional to sensed data.

34. The system of claim 31, wherein the human control unit further comprises a pedal actuatable by the human, wherein in a first position, the control unit is configured to transmit the first control signal and in a second position, the control unit is prohibited from transmitting the first control signal, wherein the first position is different from the second position.

35. : The system of claim 31, wherein the human control unit further comprises a touch device configured to be touchable by the human, wherein upon a first touch of the touch device, the control unit is configured to transmit the first control signal and upon a second touch, the control unit is prohibited from transmitting the first control signal, in particular wherein upon the first touch, information on only an axial or rotational manipulation of the second and/or third elongated medical member is transmittable and upon the second touch, information on only an axial or rotational manipulation of the second and/or third elongated medical member is not transmittable.

36. (canceled)

37. (canceled)
